(19) United States
(12) Patent Application Publication
(10) Pub. No.: US 2022/0411768 A1
(43) Pub. Date: Dec. 29, 2022

(54) METHODS OF PERFORMING RNA TEMPLATED GENOME EDITING

(71) Applicant: THE TRUSTEES OF COLUMBIA UNIVERSITY IN THE CITY OF NEW YORK, New York, NY (US)

(72) Inventors: Alejandro Chavez, New York, NY (US); Schuyler Melore, New York, NY (US)

(21) Appl. No.: 17/770,917

(22) PCT Filed: Oct. 19, 2020
(86) PCT No.: PCT/US20/56350

§ 371 Date: Apr. 21, 2022

Related U.S. Application Data
(60) Provisional application No. 62/924,050, filed on Oct. 21, 2019.

(51) Int. Cl. C12N 9/12; C12N 15/113; C12N 15/62; C12N 9/22

(52) U.S. Cl. CPC C12N 9/1276; C12N 15/113; C12N 15/62; C12N 9/22; C12Y 207/07049; C07K 2319/00; C12N 2310/20

(57) ABSTRACT

The present invention relates to in vitro genetic manipulation. In particular, it relates to RNA templated genome editing.

### RELATED APPLICATION DATA

This application claims priority to U.S. Provisional Application No. 62/924,050 filed on Oct. 21, 2019, which is hereby incorporated herein by reference in it its entirety for all purposes.

### FIELD OF THE INVENTION

The present invention relates to in vitro genetic manipulation. In particular, it relates to RNA templated genome editing.

### BACKGROUND

Gene editing is the newest frontier of biotechnology and biological research. CRISPR-Cas9 is the most well-known and widely used genetic editing technology. Indeed, genetic modification using CRISPR-Cas9 has revolutionized how we approach biological research and clinical therapeutics. The CRISPR-Cas9 system introduces specific mutations in desired locations by breaking the double-stranded helix of DNA. Specifically, CRISPR is a series of DNA sequences found in bacteria and are used to detect and destroy DNA from similar pathogens that infect the host. Cas9 is an enzyme that recognizes complementary sequences to CRISPR and cleaves them. This process makes them an attractive tool to selectively edit genes.

Indeed, while genetic modification through technology such as CRISPR-Cas9 has opened the floodgates of research and commercial applications for gene editing, there are several deficits as to the current CRISPR-Cas9 systems. For example, CRISPR-Cas9 systems create double-stranded DNA breaks, which may result in non-target small deletions or insertions, translocations and rearrangements. Therefore, not only does the CRISPR-Cas9 system potentially lead to random inserts/deletions, these non-target mutations could be potentially lethal. It is also not as efficient in non-dividing cells due to the activity of homologous recombination machinery being limited to G2 and S phases of the cell cycle.

There exists a need to eliminate the above identified short-comings.

The present invention mitigates the risk of lethal mutations by breaking just a single strand at a time for a safer, faster, and more efficient edit. The technology combines several components including a Cas9, a reverse transcriptase, and a guide RNA. The result is a technique that can be used for non-dividing cells, further expanding the applications and addressing the shortcomings of the ubiquitous CRISPR-Cas9 technology. This technology has the potential to be applied to create cell therapies, patient specific disease models for research and diagnostics, and better engineered crops and livestock.

Specifically, this technology is a strategy for creating single strand breaks in DNA to introduce point mutations for faster, more accurate genomic modifications. The system uses a Cas9 nickase (nCas9), a reverse transcriptase fused to Cas9, and an extended guide RNA (gRNA) containing an RNA template for reverse transcription that includes the desired mutations. This technology eliminates the need for the lethal double strand breaks, is more efficient at successfully introducing mutations, and can be used for non-dividing cells. It is also able to modify a longer length of sequence and more bases than the existing primer editing approach.

The present invention has several projected applications, including, personalized medicine, cellular therapy (i.e. CAR-T cell therapy, reversion of hemoglobin mutation), patient specific disease models for research, human knock-out models for research, as a research tool for study of point mutations, and genetically modified crops and livestock, but any number of other suitable applications can be envisioned.

### SUMMARY OF THE DISCLOSURE

The present disclosure is directed, at least in part, to methods and systems for precise and efficient genomic modification in any organism, independent of its intrinsic ability to perform homologous recombination. In some embodiments, the disclosure provides methods and systems for genomic modification in a high-throughput fashion without inducing potentially lethal double-stranded DNA breaks. The present disclosure provides improvements to the prime editing approach which enhance its efficacy, accuracy, length of modification and the bases that are able to be modified. The methods and systems of the disclosure can also be used for several applications, including, but not limited to, modification of cells for therapeutic use (e.g., reverting a hemoglobin mutation to wild-type), modification cells for study (e.g., production of disease models with patient specific point mutations), and production of engineered plants and animals, creating libraries of cells with one or more mutations, genome editing in both dividing and non-dividing cells, and generating random mutagenesis at a locus of interest for target gene diversification.

Accordingly, in some aspects, the present disclosure is directed to methods for modifying a target locus in a genome in a cell. In some embodiments, a Cas9 nickase (nCas9), a reverse transcriptase (RT), and an extended guide RNA (gRNA) comprising a guide RNA and an RNA template for reverse transcription that includes the desired mutations are introduced into a cell of interest (see FIG. 1A, 1B 1C). When the components are introduced into the cell, the Cas9 nickase is targeted to a genomic locus of interest by the extended gRNA. After binding to the target locus, the Cas9 nickase selectively cuts only the non-gRNA-bound (non-target) strand. As the extended gRNA contains an RNA sequence that is complementary to the cut, non-bound strand, it is able to hybridize to it. The reverse transcriptase that is fused with nCas9 then primes from the RNA-DNA hybrid formed, extending the genomic DNA from the site of the nick, using the extended gRNA as a template to introduce desired mutations into the genome (see FIG. 2A, 2B, 2C). In some embodiments, the mutation comprises a point mutation, a deletion, or an insertion. In some embodiments, the mutation comprises a deletion of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. In some embodiments, the mutation comprises an insertion of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. In some embodiments, the cell of interest is a mammalian cell. In other embodiments, the cell of interest is a plant, bacterial, or yeast cell.

To establish the functionality of the reverse transcriptase when fused to nCas9, human embryonic kidney 293T (HEK293T) cells were transfected with the nCas9-RT fusion and a reverse transcriptase template. The amount of single stranded DNA produced from the RNA template was qualified via quantitative PCR (see FIG. 3). In some embodiments, the reverse transcriptase is a human immunodeficiency virus reverse transcriptase (HIV RT). In some embodiments, the HIV RT is modified to work in mammalian cells by, for example, adding nuclear localization signals (NLS) to the HIV RT. In some embodiments, the reverse transcriptase is fused to the N-terminus, C-terminus or both termini of the Cas9 nickase. In some embodiments, the reverse transcriptase is fused to the Cas9 nickase via a linker. Exemplary RT-nCas9 fusion proteins are set forth in SEQ ID NOs: 1 and 2. In another embodiment, the reverse transcriptase is expressed separately from nCas9.

As shown in FIG. 3, the nCas9-RT fusion tested is competent for reverse transcription, and the C-terminal HIV-RT fusion to nCas9 had greater reverse transcriptase activity than the N-terminal fusion.

In order to determine whether Cas9's nuclease activity would remain intact when fused to a reverse transcriptase, a new construct containing the HIV RT fused to the C-terminus of fully nuclease-competent Cas9 was generated. The Cas9-RT fusion targeting a transfected BFP reporter was introduced into HEK293T cells, and a clear reduction in the mean BFP fluorescence was observed in cells with the Cas9-RT fusion, indicating that Cas9, when fused to an RT, is still nuclease competent (see FIG. 4).

To confirm whether the gRNA remains active after being extended with the RNA template complementary to the cut site, HEK293T cells were transfected with a series of different extended gRNAs targeted to the EMX1 locus along with fully nuclease-competent Cas9 (see FIGS. 5A and 5B). The RNA templates appended to the gRNA were designed such that they would be able to introduce a 1 base pair point mutation or a 3 base pair deletion into the EMX1 locus. As demonstrated in FIGS. 5A and 5B, the extended gRNA remained functional, and enables efficient targeting and cutting of a given locus.

The RNA template fused to the gRNA is able to efficiently complex with the nicked target DNA strand. In some embodiments, in order to increase the ease with which the RNA template is able to interact with the target strand, a linker can be added between the gRNA and RT template portions of the extended gRNA. Exemplary sequences of extended gRNAs are set forth below as SEQ ID Nos: 3-6).

In some embodiments, the methods and systems of the disclosure are modified by, for example, placing the RNA template on the 5′ end or 3′ end of the gRNA construct (see FIG. 6A). In other embodiments, the methods and systems of the disclosure are modified by utilizing alternative methods for recruiting the reverse transcriptase to the target sequence. These modifications may assist reverse transcriptase by placing it within a more sterically favorable conformation or by increasing the number of reverse transcriptase molecules brought to the complex. In some embodiments, the reverse transcriptase is directly fused to Cas9 nickase using various linkers, for example, a Gly-Ser rich or XTEN linker. In other embodiments, the reverse transcriptase is fused to Cas9 nickase using a two component system, for example, the MCP-MS2 or Suntag systems (see FIG. 6B).

In some embodiments, the reverse transcriptase is a DNA polymerase with reverse transcriptase activity, such as PolH (SEQ ID No: 7) and DinB2 (SEQ ID No. 8). In some embodiments, the reverse transcriptase is HIV reverse transcriptase (SEQ ID No: 9), Baboon endogenous virus reverse transcriptase (SEQ ID No: 10), Woolly monkey reverse transcriptase (SEQ ID No: 11), Avian reticuloendotheliosis virus reverse transcriptase (SEQ ID No: 12), Feline endogenous virus reverse transcriptase (SEQ ID No: 13), Gibbon leukemia virus reverse transcriptase (SEQ ID No: 14) or Walleye dermal sarcoma virus reverse transcriptase (SEQ ID No: 15).

In some embodiments, the reverse transcriptase is modified to promote a longer and more efficient extension of the target DNA, by, for example, ablating its RNAseH activity. The modified reverse transcriptase can re-prime if it dissociates from the template. In contrast, an RNAseH positive reverse transcriptase is expected to degrade the RNA template up until the point at which it dissociated, which may then inhibit repriming as the 3′ end may not have enough of the template RNA left to bind to it and form a stable RNA:DNA duplex for continued 3′ extension. Accordingly, in some embodiments, RNAseH mutant RTs can be utilized. In some embodiments, the methods and systems of the disclosure further employs a RNAse inhibitor, such as a ribonuclease/angiogenin inhibitor 1 (RNH1) (SEQ ID No: 16).

During the process of 3′ extension from the nicked strand, the extended DNA product may compete with the 5′ end of the DNA strand which is also bound to the template strand. In some embodiments, to help reduce competition from the 5′ DNA end, one or more DNA repair proteins, for example, 5′ flap endonucleases, e.g., FEN1 (SEQ ID No: 17), SLX1/SLX4, are recruited to cleave the native 5′ DNA strand that is competing with the 3′ extended DNA nick. In other embodiments, 5′ to 3′ exonucleases such as TAQ exonuclease domain (SEQ ID No: 18), T7 exonuclease (SEQ ID No: 19), Lambda exonuclease (SEQ ID No: 20), Polymerase A 5′ to 3′ exonuclease domain (5′ to 3′ exonuclease domain from E. coli DNA polymerase) (SEQ ID No: 21), exonuclease domain (SEQ ID No: 22) from BST DNA polymerase (SEQ ID No: 23) or BST full polymerase including the exonuclease domain (SEQ ID No: 24) are recruited to cleave the native 5′ DNA strand that is competing with the 3′ extended DNA nick.

In other embodiments, other DNA repair proteins, for example, ssDNA binding proteins, e.g., Replication Protein A (RPA), RAD51 ssDNA binding domain (SEQ ID No: 25), RAD51D ssDNA binding domain (SEQ ID No: 26), RAD51AP1 ssDNA binding domain (SEQ ID No: 27), NEQ199 ssDNA Binding protein (SEQ ID No: 28) and Single-Stranded DNA Binding Protein (SSB), are recruited to the site of extension to help stabilize the unbound 5′ DNA end and prevent its reannealing. In some embodiments, to help facilitate separation of the 5′ DNA strand from the RNA template, a 5′ to 3′ helicase with activity against RNA:DNA hybrids, e.g., PIF1 (SEQ ID No: 29), is recruited. In some embodiments, the one or more DNA repair proteins are recruited to the site of action by direct fusion to nCas9 or the reverse transcriptase. In other embodiments, the one or more DNA repair proteins are recruited to the site of action via secondary recruitment using a two component system, for example, the MCP-MS2 or Suntag systems, or any other systems similar to those listed herein.

In some embodiments, two nicks may be introduced onto the non-gRNA targeted strand. The presence of two nicks on the non-targeted strand may help disassociate it and thus lead to more efficient extension of the 3′ end by the recruited reverse transcriptase, as it no longer needs to compete with the bound strand.

In some embodiments, the methods and systems of the disclosure depend on the extended RNA containing an intact, full-length RNA template that the reverse transcriptase can use to introduce the desired mutations into the target locus. In some embodiments, in order to protect the ends of the RNA from exonucleotlytic degradation, the extended gRNA is modified, for example, by incorporating sequences within the extended gRNA from Kaposi's sarcoma-associated herpesvirus (KSHV) or from the Flavivirus family, that block 3′ to 5′ or 5′ to 3′ exonuclease activity, respectively. These sequences protect the template extensions from degradation by endogenous exonucleases and increase the efficiency of targeted genome modification. In some embodiments, a structural viral sequence is added to the 5′ or the 3′ end of the extended gRNA to block either Xrn1 or exosome-mediated degradation of the extended gRNA (see FIG. 6C). In other embodiments, an exonuclease blocking sequence is used to block degradation of the extended gRNA.

In some embodiments, the desired mutations are introduced downstream of the nick site by extending from the 3′ nick site. In other embodiments, the desired mutations are introduced upstream of the nick site, by, for example, using a high fidelity reverse transcriptase with a 3′ to 5′ proofreading activity, e.g., DNA polymerase RTX (SEQ ID No: 30). The DNA polymerase RTX is capable of performing RNA-templated DNA synthesis and has preserved the 3′ to 5′ exonuclease activity. Using a reverse transcriptase with proofreading activity also increases the fidelity with which targeted genomic modification is made. In some embodiments, the high fidelity reverse transcriptase is M160 reverse transcriptase (SEQ ID No: 31), MMULV reverse transcriptase (SEQ ID No: 32), MAGMA DNA polymerase (SEQ ID No: 33) or Foamy virus reverse transcriptase (SEQ ID No: 34).

In another aspect, the present disclosure is directed to methods for creating libraries of cells with one or more mutations. In some embodiments, the mutation comprises a mutation, e.g., a point mutation, a deletion, or an insertion. In some embodiments, the mutation comprises a deletion of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. In some embodiments, the mutation comprises an insertion of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. In other embodiments, libraries of cells can be created, each with a different mutation, by performing a low MOI transduction of the gRNA-template construct, such that each cell receives at most one.

In another aspect, the present disclosure is directed to methods for genome editing in non-dividing cells. In some embodiments, the methods do not require homologous recombination machinery.

The present disclosure is also directed, at least in apart, to methods of generating random mutagenesis at a locus of interest. In some embodiments, the methods and systems of the disclosure are useful for target gene diversification. In some embodiments, the methods and systems of the disclosure employ a naturally error-prone reverse transcriptase, e.g., a reverse transcriptase from diversity generating retroelements (DGR) within various bacteria and phages, e.g., Bordetella bacteriophage reverse transcriptase (Brt) gene (SEQ ID No: 35), Treponema DGR reverse transcriptase gene (SEQ ID No: 36), Bacteroides DGR reverse transcriptase gene (SEQ ID No: 37) and Eggerthella lenta DGR reverse transcriptase gene (SEQ ID No: 38). In some embodiments, the methods and systems of the disclosure employ a synthetic, more mutagenic reverse transcriptase variant. In other embodiments, the methods and systems of the disclosure involve recruitment of an enzyme to the Cas9-RT complex with the ability to mutagenize the RNA template, or change the RNA bases to a substrate that the reverse transcriptase is more error-prone in reading. In some embodiments, the enzyme is ADAR. In some embodiments, the RNA base can be 3-methylcytosine.

In some embodiments, the methods and systems of the disclosure employ a protein destabilization domain that causes proteins containing it to be actively destroyed during the S and G2/M phases of the cell cycle, such as the CDT degron (SEQ ID No: 39). One concern with using a Cas9 nickase, which is required for the Cas9-RT system, is that the nick if present during S-phase can lead to a double strand break. This double strand break then creates the opportunity for small insertions and deletions to occur within the target locus which not only limit the ability of this system to perform precise modifications but also may create undesired deleterious repair events (e.g., introduction of a premature stop codon or a frame shift mutation). The fusion of the CDT degron, in one or two copies (SEQ ID No: 40), to the Cas9-RT enzyme renders it only stable during G0/G1 and in doing so reduces the rate of undesired repair events as now nicks will only be present during G0/G1.

In some embodiments, the methods and systems of the disclosure employ a single-chain antibody that binds to RNA-DNA hybrids, such as the scFV S9.6 protein (SEQ ID No: 41). The presence of the scFV S9.6 protein would stabilize the Cas9-RT complex between the RNA template fused to the gRNA and the target DNA strand it invades into and thereby allow more time for the reverse transcriptase to function and thus increase the rate of programmed genetic alterations.

In some embodiments, the methods and systems of the disclosure employ domains or full length proteins that have previously been shown to assist in helping the proteins they are fused to fold and remain in solution, such as Protein G B1 domain (GB1) (SEQ ID No: 42), Maltose Binding Protein (MBP) (SEQ ID No: 43), and Thioredoxin (TRXA) (SEQ ID No: 44). As many components in the system of this disclosure are complex and composed of multiple protein domains (e.g., Cas9 and a reverse transcriptase), fusion of these domains to the Cas9-RT system would increase its activity by maintaining it in the active soluble state by preventing protein misfolding.

In some embodiments, the methods and systems of the disclosure employ a single-chain antibody that binds to RNA-DNA hybrids fused to GB1 solubilization domain, such as scFV S9.6 GB1 fusion (SEQ ID No: 45).

In some embodiments, the methods and systems of the disclosure employ a double stranded DNA binding protein, such as SSO7D (SEQ ID No: 46), to help increase the dwell time of the Cas9-RT fusion onto DNA and thereby provide more opportunities for the reverse transcriptase to extend itself off of the RNA template and introduce the desired modifications into the genome.

In some embodiments, the methods and systems of the disclosure employ a C-to-U editing enzymes, such as ADAR1 (SEQ ID No: 47), ADAR2 (SEQ ID No: 48), rat apolipoprotein B mRNA editing enzyme, catalytic polypeptide-like 1 (rAPOBEC) (SEQ ID No: 49), and Activation-induced cytidine deaminase (AID) (SEQ ID No: 50), to introduce changes to the template RNA fused in cis to the gRNA which will then be used by the reverse transcriptase to modify the target locus. As each cell will contain many copies of the gRNA each with different changes to the template region driven by these base modifying proteins, a large amount of diversity can be created within a target region.

In conclusion, the present disclosure provides methods and systems for creating programmed precise genomic modification within mammalian cells in a high-throughput fashion without inducing potentially lethal double-stranded DNA breaks. The methods and systems of the disclosure can also be used for several applications, including, but not limited to, modification of cells for therapeutic use (e.g., reverting a hemoglobin mutation to wild-type), modification cells for study (e.g., production of disease models with patient specific point mutations), and production of engineered plants and animals, creating libraries of cells with one or more mutations, genome editing in non-dividing cells, and generating random mutagenesis at a locus of interest for target gene diversification.

Disclosed herein are systems and methods for RNA templated genome editing.

Accordingly, in a first aspect, the present invention provides a method for modifying a target locus in a genome in a cell, comprising introducing into the cell: a Cas9 nickase (nCas9), a reverse transcriptase (RT), and an extended guide RNA (gRNA), wherein the extended gRNA comprises a guide RNA and an RNA template for the RT; wherein the extended gRNA binds to a DNA strand at the target locus in the genome; and wherein the RNA template comprises a desired mutation to be introduced into the target locus, thereby modifying the target locus in the genome.

In various embodiments of the first aspect of the invention delineated herein, the method does not induce double-stranded DNA breaks.

In various embodiments of the first aspect of the invention delineated herein, the Cas9 nickase nicks a DNA strand that is not bound by the extended gRNA.

In various embodiments of the first aspect of the invention delineated herein, the Cas9 nickase introduces two nicks onto the DNA strand that is not bound by the extended gRNA.

In various embodiments of the first aspect of the invention delineated herein, the RNA template hybridizes to the DNA strand that is not bound by the extended gRNA to form a RNA/DNA hybrid.

In various embodiments of the first aspect of the invention delineated herein, the reverse transcriptase primes from the RNA/DNA hybrid and extends the DNA strand based on the RNA template in the extended gRNA to introduce the desired mutation into the target locus.

In various embodiments of the first aspect of the invention delineated herein, the desired mutation is introduced upstream of a nick introduced by the Cas9 nickase.

In various embodiments of the first aspect of the invention delineated herein, the reverse transcriptase has preserved 3′ to 5′ exonuclease activity to enable the desired mutation to be introduced upstream of the 3′ nick.

In various embodiments of the first aspect of the invention delineated herein, the desired mutation is introduced downstream of a nick introduced by the Cas9 nickase.

In various embodiments of the first aspect of the invention delineated herein, the reverse transcriptase is an error prone reverse transcriptase which diversifies a DNA region of interest.

In various embodiments of the first aspect of the invention delineated herein, the reverse transcriptase is a human immunodeficiency virus reverse transcriptase (HIV RT).

In various embodiments of the first aspect of the invention delineated herein, the reverse transcriptase is fused to the N-terminus or the C-terminus of the Cas9 nickase.

In various embodiments of the first aspect of the invention delineated herein, the reverse transcriptase is fused to the Cas9 nickase via a linker.

In various embodiments of the first aspect of the invention delineated herein, the linker is a Gly-Ser rich linker or an XTEN linker.

In various embodiments of the first aspect of the invention delineated herein, the RNA template is fused to either the 5′ end or the 3′ end of the guide RNA.

In various embodiments of the first aspect of the invention delineated herein, the RNA template is fused to the guide RNA via a linker.

In various embodiments of the first aspect of the invention delineated herein, the desired mutation comprises a point mutation, an insertion, or a deletion.

In various embodiments of the first aspect of the invention delineated herein, a DNA repair protein is recruited during extension of the DNA strand at the target locus.

In various embodiments of the first aspect of the invention delineated herein, the extended gRNA further comprises sequences that block exonuclease activity.

In various embodiments of the first aspect of the invention delineated herein, the cell is a mammalian cell.

### BRIEF DESCRIPTION OF THE FIGURES

FIGS. 1A, 1B, and 1C depict components of the system of the disclosure. FIG. 1A) Plasmid encoding Cas9 H840A nickase (nCas9) which nicks the non-target DNA strand. FIG. 1B) Plasmid encoding the reverse transcriptase (RT). The RT may be fused to the N- or C-terminus of nCas9 or may be expressed separately. FIG. 1C) Plasmid expressing the gRNA-template construct. This comprises a guide RNA (gRNA) targeting the locus of interest as well as another sequence downstream of the gRNA tail that is complementary to the non-target genomic DNA strand and contains mutations to be introduced (shown as a star here).

FIGS. 2A, 2B, and 2C depict the process by which mutations are introduced to the genome. FIG. 2A) nCas9 targets to the locus of interest via the extended gRNA-RT template construct. nCas9 nicks the non-target genomic DNA strand. FIG. 2B) The RNA template hybridizes to the non-target DNA strand. FIG. 2C) The RT then primes from the RNA-DNA hybrid created by the template hybridizing to the cut target and polymerizes from the nick to introduce mutations contained in the RNA template into the target DNA locus. Here, a small insertion has been introduced, which is shown in the edited locus.

FIG. 3 depicts production of ssDNA by nCas9-HIV RT fusions. 293T Cells were transfected with nCas9-HIV RT Fusions and an RNA reporter for HIV RT activity that will result in ssDNA production in the presence of HIV RT. Negative controls were transfected with iRFP instead of RT. Data are shown as the mean±s.e.m (n=2 independent transfections).

FIG. 4 illustrates that nCas9-HIV RT fusion retains cutting activity. Cells were transfected with a BFP Reporter plasmid, a gRNA against the BFP plasmid, and an nCas9-HIV RT fusion. BFP geometric mean fluorescence intensity (a.u.) drops to 54% in the presence of the nCas9-HIV RT construct. Data are shown as the mean±s.e.m (n=2 independent transfections).

FIGS. 5A and 5B depict editing efficiencies of gRNA-Template constructs at the EMX1 locus. HEK293T cells were transfected with Cas9 and either a gRNA without a template (“regular gRNA”), a gRNA-template construct with homology to the EMX1 locus seeking to introduce one of three mutations, or a gRNA-template construct where the template has no homology to the EMX1 locus. The gRNA without Cas9 (“gRNA alone”) was transfected as a negative control. FIG. 5A) Amount of editing at the EMX1 locus induced by each gRNA construct as determined by next generation sequencing and the Amplican indel analysis package. Data are shown as the mean±s.e.m (n=2 independent transfections) FIG. 5B) Amount of frameshift mutations at the EMX1 locus induced by each gRNA construct as determined by next generation sequencing and the Amplican software package. Data are shown as the mean±s.e.m (n=2 independent transfections).

FIGS. 6A, 6B, and 6C depict optimization of the system of the disclosure. FIG. 6A) The effect of placing the template region of the gRNA-template construct on the 5′ vs. 3′ end of the construct. FIG. 6B) The effect of using an nCas9-HIV RT fusion vs. recruiting HIV RT to the locus via the MCP-MS2 system. FIG. 6C) Addition of structured viral sequences to the 5′ or 3′ end of the gRNA-template construct to block either Xrn1 or Exosome-mediated degradation of the gRNA-template.

### DETAILED DESCRIPTION

### Definitions

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein an “antibody” refers to IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria. In another example, an antibody includes two heavy (H) chain variable regions and two light (L) chain variable regions. It should be noted that a VH region (e.g. a portion of an immunoglobulin polypeptide is not the same as a VH segment, which is described elsewhere herein). The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

As described herein, an “antigen” is a molecule that is bound by a binding site on an antibody. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

“Binding” as used herein (e.g. with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “complexing” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

Binding region” as used herein refers to the region within a nuclease target region that is recognized and bound by the nuclease.

The term “Cas protein” as used herein describes CRISPR-associated protein, which is an RNA-guided endonuclease that is directed towards a desired genomic target when complexed with an appropriately designed small guide RNA (“gRNA”). An example of a Cas protein is Cas9 which is CRISPR-associated protein 9. gRNAs comprise approximately a 20-nucleotide sequence (the protospacer), which is complementary to the genomic target sequence. Next to the genomic target sequence is a 3′ protospacer-associated motif (“PAM”), which is required for Cas9 binding. In the case of Streptococcus Pyogenes Cas9 (SpCas9), this has the sequence NGG. Other sequences are as described herein and as known in the art. In some embodiments, upon binding the DNA target, Cas9 cleaves both strands of DNA, thereby stimulating repair mechanisms that can be exploited to modify the locus of interest. In some embodiments, the Cas9 protein is mutated to convert Cas9 into a nicking enzyme, otherwise referred to as Cas9 nickase, which generates single-strand nicks in DNA.

A “Cas9 nickase” may be interchangeably referred to “nCas9” or “Cas9n”. Methods for generating Cas9 proteins (or fragments thereof) having a mutated nicking function are known (eg, Jinek et al., Science. 337: 816-821 (2012); Qi et al., “Repurposing CRISPR as an RNA-Guided Platform for Sequence-Specific Control of Gene Expression” (2013) Cell. 28; 152 (5): 1173-83. The entire contents of each are incorporated herein by reference). For example, the DNA cleavage domain of Cas9 is known to include two subdomains, the HNH nuclease subdomain and the RuvC1 subdomain. The HNH subdomain cleaves a strand complementary to gRNA, whereas the RuvC1 subdomain cleaves a non-complementary strand. Mutations within these subdomains can modify the nuclease activity of Cas9. In some embodiments, inactivation of one or domain with preservation of the other results in nickase activity. For example, the RuvC domain is preserved and the HNH domain is mutated to obtain nickase enzyme activity. Mutated Cas9 proteins include, D10A, N863A and H840A Cas9 nickases and the like. (Jinek et al., Science. 337: 816-821 (2012); Qi et al., Cell. 28; 152 (5): 1173-83 (2013)). In some embodiments, a protein comprising a fragment of Cas9 is provided. For example, in some embodiments, the protein comprises one of two Cas9 domains: (1) a Cas9 gRNA binding domain; or (2) a Cas9 DNA cleavage domain. In some embodiments, a protein comprising Cas9 or a fragment thereof is referred to as a “Cas9 variant”. Cas9 variants share homology with Cas9 or fragments thereof.

“Cleave” or “cleavage” as used herein means the act of breaking the covalent sugar-phosphate bond between two adjacent nucleotides within a polynucleotide. In the case of a double-stranded polynucleotide, a covalent sugar-phosphate bond on both strands will be broken, unless otherwise specified.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can Watson-Crick (e.g., A-T/U and C-G) or Hoogsteen base pair between nucleotides or nucleotide analogs of nucleic acid molecules. “Complementarity” refers to a property shared between two nucleic acid sequences, such that when they are aligned antiparallel to each other, the nucleotide bases at each position will be complementary.

“Donor vector”, “donor template” and “donor DNA” as used interchangeably herein refers to a double-stranded DNA fragment or molecule that includes the insert being introduced into the genomic DNA. The donor vector may encode a fully-functional protein, a partially-functional protein or a short polypeptide. The donor vector may also encode an RNA molecule.

The terms “engineered”, “constructed” or “designed” as used interchangeable herein, refers to the aspect of having been manipulated by the hand of man. As is common practice and is understood by those in the art, progeny and copies of an engineered polynucleotide (and/or cells or animals comprising such polynucleotides) are typically still referred to as “engineered” even though the actual manipulation was performed on a prior entity.

The term “extended gRNA” or “extended guide RNA” as used interchangeably herein refers to a complex that comprises of two or more RNA species. For example, an extended guide RNA comprises a “guide RNA” and an “RNA template” as described in further detail herein. The term “guide RNA” as used interchangeably with “gRNAs” herein may be referred to as “single-guide RNAs” (“sgRNAs”) and is used to described Cas protein associated guide RNA's for CRISPR-Cas systems. CRISPR-Cas mammalian systems may be generated through methods known in the art, for example as described in Nageshwaran, S., et al. (2018). CRISPR Guide RNA Cloning for Mammalian Systems. Journal of Visualized Experiments, (140). doi:10.3791/57998, the entirety of which is incorporated by reference. Typically, gRNAs that exist as single gRNA species comprise two domains: (1) a domain that shares homology to a target nucleic acid (e.g., and directs binding of a Cas protein complex to the target); and (2) a domain that binds a Cas protein. In some embodiments, gRNAs that exist as an extended gRNA may comprise two or more of domains (1) or (2) or both. In some embodiments, such extended gRNAs further comprise one or more RNA templates as described in further detail herein.

Functional” and “full-functional” as used herein describes protein that has biological activity. A “functional gene” refers to a gene transcribed to mRNA, which is translated to a functional protein.

“Genetic construct” as used herein refers to the DNA or RNA molecules that comprise a nucleotide sequence that encodes a protein or an RNA molecule. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genome editing” as used herein refers to changing a gene. Genome editing may include correcting or restoring a mutant gene. Genome editing may include knocking out a gene, such as a mutant gene or a normal gene. Genome editing may be used to introduce a label onto a protein.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent Identity may be performed manually or by using a computer sequence algorithm such as BLAST or BLAST 2.0.

The terms “increased”, “increase”, “enhance”, or “activate” optionally used with the term “substantially” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or a reporter, an “increase” is a statistically significant increase in such level. In the context of a protein or enzyme, an “increase” is a statistically significant increase in such level. In some embodiments, the reference is the corresponding wild type or un-mutated version of the protein or enzyme.

The terms “inhibit”, “reduce”, “decrease”, “deactivate” optionally used with the term “substantially” are all used herein to mean a decrease by a statically significant amount. In some embodiments, the terms ““inhibit”, “reduce”, “decrease”, “deactivate” can mean a decrease of at least 2%, as compared to a reference level, for example a decrease of at least about 5%, at least about 7.5%, at least about 10%, at least about 15%, at least about 20%, at least about 25%, or at least about 30%, or at least about 40%, or at least about 50%, or at least about 60%, or at least about 70%, or at least about 80%, or at least about 90% or up to and including a 100% decrease or any increase between 2-100% as compared to a reference level, or at least about a 2-fold, or at least about a 3-fold, or at least about a 4-fold, or at least about a 5-fold or at least about a 10-fold decrease, or any increase between 2-fold and 10-fold or greater as compared to a reference level. In the context of a marker or a reporter, “decrease” is a statistically significant decrease in such activity level. In the context of a protein or enzyme, a “decrease” is a statistically significant decrease in such activity level. In some embodiments, the reference is the corresponding wild type or un-mutated version of the protein or enzyme.

“Mismatch” as used herein means a nucleotide cannot form a Watson-Crick (e.g., A-T/U and C-G) or Hoogsteen base pair with another nucleotide on the opposite strand of a double-stranded polynucleotide or with another nucleotide from a different polynucleotide.

Mutation. As used herein, the term “mutation” or “mutant” indicates a change or changes introduced in a wild type DNA sequence or a wild type amino acid sequence. Examples of mutations include, but are not limited to, substitutions, insertions, deletions, and point mutations. Mutations can be made either at the nucleic acid level or at the amino acid level.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that can introduce random micro-insertions and micro-deletions (indels) at the DNA breakpoint This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

As used herein, the term “nuclear localization signals” or “NLS” refers to a peptide, or derivative thereof, that directs the transport of an expressed peptide, protein, or molecule associated with the NLS; from the cytoplasm into the nucleus of the cell across the nuclear membrane.

The terms “nucleic acid” or “oligonucleotide” or “polynucleotide” as used interchangeably herein means at least two nucleotides upwards of any length, either ribonucleotides or deoxyribonucleotides, covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or hybrids, or a polymer, where the nucleic acid may contain combinations of deoxyribo- and ribo-nucleotides, and combinations of bases including uracil, adenine, thymine, cytosine, guanine, inosine, xanthine hypoxanthine, isocytosine, isoguanine, purine and pyrimidine bases or other natural, chemically or biochemically modified, non-natural, or derivatized nucleotide bases. Nucleic acids may be obtained by chemical synthesis methods or by recombinant methods. “Oligonucleotide” generally refers to polynucleotides of between about 3 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

As used herein “operably linked” means that a nucleic acid element is positioned so as to influence the initiation of expression of the polypeptide encoded by the structural gene or other nucleic acid molecule. For example, “operably linked” means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Operably linked.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “plurality” as used herein means a number greater than one.

“Promoter” as used herein means a synthetic or naturally-derived nucleic acid sequence which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

“Reading frame”, “Open Reading Frame” or “Coding Frame” as used herein interchangeably means a grouping of three successive bases in a sequence of DNA that potentially constitutes the codons for specific amino acids during translation into a polypeptide.

As used herein, the term “reverse transcriptase” refers to a protein, enzyme, polypeptide, or polypeptide fragment capable of producing DNA from an RNA template. For example, the term “reverse transcriptase” refers to an enzyme with RNA-dependent DNA polymerase activity, with or without the usually associated DNA-dependent DNA polymerase and ribonuclease activity observed with wild-type reverse transcriptases.

Reverse Transcriptase Activity. As used herein, the term “reverse transcriptase activity,” “reverse transcription activity,” or “reverse transcription” indicates the capability of an enzyme to synthesize DNA strand (that is, complementary DNA or cDNA) using RNA as a template or the process thereof.

As used herein the term “sequence-specific nuclease” refers to programmable nucleases that enable genome editing by cleaving DNA at specific genomic loci, signaling DNA damage and recruiting endogenous repair machinery for either NHEJ or HDR to the cleaved site to mediate genome editing. Sequence-specific nucleases can be endonucleases, exonuclease, or both. The term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” The methods and compositions described herein may be applied to cleavage sites generated by endonucleases. In some alternatives of the system, the system can further provide nucleic acids that encode an endonuclease, such as CRISPR-associated protein (Cas), an Argonaute protein (AGO), TAL Effector Nuclease” (TALEN), or a meganuclease such as MegaTAL, or a fusion protein comprising a domain of an endonuclease, for example, Cas9, Ago, TALEN, or MegaTAL, or one or more portion thereof. Ago is a These examples are not meant to be limiting and other endonucleases and alternatives of the system and methods comprising other endonucleases and variants and modifications of these exemplary alternatives are possible without undue experimentation. All such variations and modifications are within the scope of the current teachings. The term “exonuclease” refers to enzymes that cleave phosphodiester bonds at the end of a polynucleotide chain via a hydrolyzing reaction that breaks phosphodiester bonds at either the 3′ or 5′ end. The polynucleotide may be double-stranded DNA (dsDNA), single-stranded DNA (ssDNA), RNA, double-stranded hybrids of DNA and RNA, and synthetic DNA (for example, containing bases other than A, C, G, and T). The term “5′ exonuclease” refers to exonucleases that cleave the phosphodiester bond at the 5′ end. The term “3′ exonuclease” refers to exonucleases that cleave the phosphodiester bond at the 3′ end. Exonucleases may cleave the phosphodiester bonds at the end of a polynucleotide chain at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolosis and chemotherapy agents. Exonucleases may cleave the phosphodiester bonds at blunt ends or sticky ends. E. coli exonuclease I and exonuclease III are two commonly used 3 ‘-exonucleases that have 3’-exonucleolytic single-strand degradation activity. Other examples of 3 ‘-exonucleases include Nucleoside diphosphate kinases (NDKs), NDK1 (NM23-H1), NDK5, NDK7, and NDK8 (Yoon J-H, et al., Characterization of the 3’ to 5′ exonuclease activity found in human nucleoside diphosphate kinase 1 (NDK1) and several of its homologues. (Biochemistry 2005:44(48): 15774-15786), WRN (Ahn, B., et al., Regulation of WRN helicase activity in human base excision repair. J. Biol. Chem. 2004, 279: 53465-53474) and Three prime repair exonuclease 2 (Trex2) (Mazur, D. J., Perrino, F. W., Excision of 3′ termini by the Trex1 and TREX2 3′→5′ exonucleases. Characterization of the recombinant proteins. J. Biol. Chem. 2001, 276: 17022-17029; both references incorporated by reference in their entireties herein). E. coli exonuclease VII and T7-exonuclease Gene 6 are two commonly used 5′-3′ exonucleases that have 5% exonucleolytic single-strand degradation activity. The exonuclease can be originated from prokaryotes, such as E. coli exonucleases, or eukaryotes, such as yeast, worm, murine, or human exonucleases. In some alternatives of the systems provided herein, the systems can further comprise an exonuclease or a vector or nucleic acid encoding an exonuclease. In some alternatives, the exonuclease is Trex2. In some alternatives of the methods provided herein, the methods can further comprise providing exonuclease or a vector or nucleic acid encoding an exonuclease, such as Trex2

“Target gene” as used herein refers to any nucleotide sequence encoding a known or putative gene product.

The term “target site” is used herein to refer to the specific locus of the target gene on a genome.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art, such as in Kyte et al, J. Mol. Biol. 157: 105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode an mutation and/or at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular. Moreover, unless otherwise stated, the present invention was performed using standard procedures.

### RNA Templated Genome Editing

According to some embodiments, the present invention is directed to systems and methods for modifying a target locus in a genome in a cell, comprising:

introducing into the cell: a Cas9 nickase (nCas9), a reverse transcriptase (RT), and an extended guide RNA (gRNA), wherein the extended gRNA comprises a guide RNA and an RNA template for the RT;

wherein the extended gRNA binds to a DNA strand at the target locus in the genome; and

wherein the RNA template comprises a desired mutation to be introduced into the target locus,

thereby modifying the target locus in the genome.

According to some embodiments, the present invention comprises the use of one or more nucleic acid, polynucleotide, or oligonucleotide coding sequences, the foregoing terms being used interchangeably herein. According to some embodiments, the present coding sequences are introduced into a genome, chromosome, and etc. According to some embodiments, the present sequences encode for functional genes or proteins as used by the methods and systems described herein. According to some embodiments, the present sequences encode for the present system, components or subcomponents, such as a Cas9 nickase (nCas9), a reverse transcriptase (RT), an extended guide RNA (gRNA), a guide RNA, an RNA template for the RT extended guide RNA(s), a desired mutation(s), and the like, or any combination thereof.

The nucleic acid, poly or oligonucleotides which encode for sequences described herein may be synthesized or obtained from commercial sources. Synthesis of nucleic acid sequences is known in the art and can be by any means, including array synthesis, PCR, solid phase synthesis, or recombinant synthesis.

According to some embodiments, the present invention comprises the use of one or more peptide(s), polypeptide(s), protein(s), or fragment thereof the foregoing terms being used interchangeably herein. According to some embodiments, the present proteins comprise functional proteins as used by the methods and systems described herein. According to some embodiments, the present proteins as used in the present system, method, components or subcomponents, comprise a Cas9 nickase (nCas9), a reverse transcriptase (RT), an extended guide RNA (gRNA), a guide RNA, an RNA template for the RT extended guide RNA(s), a desired mutation(s), and the like, or any combination thereof.

### Cas9 Nickase

According to some embodiments, the present invention comprises a sequence-specific nuclease or at least one nucleic acid sequence encoding a sequence-specific nuclease. In some embodiments, the nucleic acid-guided sequence-specific nuclease forms a complex with the 3′ end of a gRNA. The specificity of the presently described system depends on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the nucleic acid-guided sequence-specific nuclease can be directed to new genomic targets. The PAM sequence is located on the DNA to be cleaved and is recognized by a nucleic acid-guided sequence-specific nuclease. PAM recognition sequences of the nucleic acid-guided sequence-specific nuclease can be species specific.

Exemplary sequence-specific nucleases for use in the present invention include, but are not limited to, Cas, Cas9, Cas12, Clas13, AGO, PfAGO, NgAgo, TALEN, or MegaTAL. According to some embodiments, the sequence-specific nuclease is a Cas protein. According to some embodiments, the Cas nuclease is a Cas9 protein.

In some embodiments, the Cas9 protein is derived from a bacterial genus of Streptococcus, Staphylococcus, Brevibacillus, Corynebacter, Sutterella, Legionella, Francisella, Treponema, Filifactor, Eubacterium, Lactobacillus, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter. In some embodiments, the Cas9 protein is selected from the group, including, but not limited to, Streptococcus pyogenes, Francisella novicida, Staphylococcus aureus, Neisseria meningitides, Streptococcus thermophiles, Treponema denticola, Brevibacillus laterosporus, Campylobacter jejuni, Corynebacterium diphtheria, Eubacterium ventriosum, Streptococcus pasteurianus, Lactobacillus farciminis, Sphaerochaeta globus, Azospirillum, Gluconacetobacteriazotrophicus, Neisseria cinerea, Roseburia intestinalis, Parvibaculum lavamentivorans, Nitratifractor salsuginis, and Campylobacter lari.

According to some embodiments, the Cas protein is a Cas9 ortholog selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum.

In some embodiments, the Cas9 protein is selected from the group including, but not limited to, Streptococcus pyogenes Cas9 (SpCas9), a Francisella novicida Cas9 (FnCas9), a Staphylococcus aureus Cas9 (SaCas9), Neisseria meningitides Cas9 (NmCas9), Streptococcus thermophiles Cas9 (StCas9), Treponema denticola Cas9 (TdCas9), Brevibacillus laterosporus Cas9 (BlatCas9), Campylobacter jejuni Cas9 (CjCas9), a variant endonuclease thereof, or a chimera thereof. In some embodiments, the Cas9 endonuclease is a SpCas9 variant, a SaCas9 variant, or a StCas9.

The Cas protein complex unwinds a DNA duplex and searches for sequences complementary to the gRNA and the correct PAM. The Cas protein only mediates cleavage of the target DNA if both conditions are met. By specifying the type Cas-based nuclease and the sequence of one or more gRNA molecules, DNA cleavage sites can be localized to a specific target domain Given that PAM sequences are variant and species specific, target sequences can be engineered to be recognized by only certain Cas9-based proteins. In some embodiments, the Cas9 protein can recognize a PAM sequence YG, NGG, NGA, NGCG, NGAG, NGGNG, NNGRRT, NNGRRT, NNNRRT. NAAAAC, NNNNGNNT, NNAGAAW, NNNNCNDD, or NNNNRYAC.

According to some embodiments, the Cas9 protein is a Cas9 nickase that lacks or lacks one of two catalytic sites for endonuclease activity (RuvC and HNH), and endonuclease activity. According to some embodiments, a nickase may be a Cas9 nickase having a mutation at a position corresponding to D10A of S. pyogenes Cas9; having a mutation at a position corresponding to H840A of the Streptococcus pyogenes Cas9); or other mutation as necessary so that the Cas9 protein exhibits nickase activity.

According to some embodiments, the Cas9 nickase comprises cutting activity of the target strand. According to some embodiments, the Cas9 nickase comprises cutting activity of the non-target strand. According to some embodiments, the Cas9 D10A nickase comprises cutting activity of the target strand. According to some embodiments, the Cas9 H840A nickase comprises cutting activity of the non-target strand.

According to some embodiments, a nick results in homology directed repair. According to some embodiments, repair of a nick does not require homologous recombination machinery.

According to some embodiments, one nick is introduced into the non-targeted strand. According to some embodiments, more than one nick is introduced into the non-targeted strand. According to some embodiments, a plurality of nicks are introduced into the non-targeted strand. According to some embodiments, two nicks are introduced into the non-targeted strand.

According to some embodiments, the nuclease activity of the Cas9 protein is preserved. According to some embodiments, the present invention further comprises a reverse transcriptase. According to some embodiments, the reverse transcriptase is fused to a Cas9 protein. According to some embodiments, the nuclease activity of the Cas9 protein is preserved when a reverse transcriptase is fused to the Cas9 protein.

### Reverse Transcriptase

According to some embodiments, the present invention comprises a reverse transcriptase or sequence(s) encoding a reverse transcriptase.

Reverse transcriptases for use in the systems and methods of the invention include any enzyme or polypeptide having reverse transcriptase activity. Such enzymes include, but are not limited to, retroviral reverse transcriptases, such as retroviral reverse transcriptase, retrotransposon reverse transcriptase, bacterial reverse transcriptase, and etc; DNA polymerase, such as Tth DNA polymerase, Taq DNA polymerase, Tne DNA polymerase, Tma DNA polymerase and etc; and the like; and mutants, fragments, variants or derivatives thereof. Enzymes with reverse transcriptase activity is as known and described in the field, for example in Saiki, R. K., et al., Science 239:487-491 (1988); U.S. Pat. Nos. 4,889,818 and 4,965,188; WO 96/10640; U.S. Pat. Nos. 5,374,553; 5,948,614 and 6,015,668, which are incorporated by reference herein in their entireties.

According to some embodiments, the reverse transcriptase is expressed as fused with the Cas protein. According to some embodiments, the reverse transcriptase is expressed as fused with the Cas9 nickase. According to some embodiments, the reverse transcriptase is expressed separately from the Cas protein. According to some embodiments, the reverse transcriptase is fused with the Cas protein. According to some embodiments, the reverse transcriptase is fused to the Cas protein. According to some embodiments, the reverse transcriptase is fused to the C-terminus of the Cas protein, the N-Terminus of the Cas protein, or both. According to some embodiments, the reverse transcriptase is fused to the C-terminus of the Cas protein.

According to some embodiments, the present invention comprises alternative methods for recruiting proteins with reverse transcriptase activity to the target sequence. Alternative methods include altering steric conformation, increasing the number of molecules with reverse transcriptase activity or both. According to some embodiments, the reverse transcriptase is fused directly to the Cas protein.

According to some embodiments, the reverse transcriptase is fused to the Cas protein via a linker. Preferred examples of a linker include a Gly-Ser linker or XTEN linker. According to some embodiments, the reverse transcriptase is fused to the Cas9 protein using a two component system. Preferred examples of a two component system include the MCP-MS2 or Suntag systems, the systems of which are well known in the art and incorporated herein. Reverse transcriptase proteins as expressed fused to a Cas protein is referred to herein as an RT-Cas fusion protein. A specific example is a RT-Cas9 fusion protein. Exemplary RT-nCas9 fusion proteins are set forth in SEQ ID NOs: 1 and 2.

According to some embodiments, the reverse transcriptase is a DNA polymerase with reverse transcriptase activity. Preferred examples of DNA polymerases with reverse transcriptase activity includes POLH and DinB2. Exemplary sequences are set forth in SEQ ID Nos: 7-8.

According to some embodiments, examples of reverse transcriptases include retroviral reverse transcriptases such as Maloney Murine Leukemia Virus (M-MLV) reverse transcriptase, Human Immunodeficiency Virus (HIV) reverse transcriptase, Rous sarcoma virus (RSV) reverse transcriptase, Avian Myeloblastosis Virus (AMV) reverse transcriptase, Rous-associated virus (RAV) reverse transcriptase, and Myeloblastosis Associated Virus (MAV) reverse transcriptase or other Avian sarcoma leukosis virus (ASLV) reverse transcriptases. Additional reverse transcriptases which may be mutated to make the reverse transcriptases of the invention include bacterial reverse transcriptases (e.g., Escherichia coli reverse transcriptase) (see, e.g., Mao et al., Biochem. Biophys. Res. Commun. 227:489-93 (1996)) and reverse transcriptases of Saccharomyces cerevisiae (e.g., reverse transcriptases of the Tyl or Ty3 retrotransposons) (see, e.g., Cristofari et al., Jour. Biol. Chem. 274:36643-36648 (1999); Mules et al., Jour. Virol. 72:6490-6503 (1998)). Other reverse transcriptases that can be used in accordance with the described invention include, but are not limited to reverse transcriptases isolated from viruses isolated from, for example, baboon, fowl pox, monkey, feline, gibbon, koala bear, and wild boar species. Preferred reverse transcriptases include HIV reverse transcriptase, Baboon endogenous virus reverse transcriptase, Woolly monkey reverse transcriptase, Avian reticuloendotheliosis virus reverse transcriptase, Feline endogenous virus reverse transcriptase, Gibbon leukemia virus reverse transcriptase or Walleye dermal sarcoma virus reverse transcriptase. Exemplary sequences are as set forth in SEQ ID Nos: 9-15.

According to some embodiments, the reverse transcriptase is modified to have reduced, substantially reduced, or lacking in RNase H activity. Modifications of RNAseH activity as described in the context of the RNA template herein, comprises the ability to promote longer and more efficient extension of the target DNA, the ability to re-prime if disassociated from the template, or both. Such enzymes that are reduced or substantially reduced in RNase H activity include RNase H− derivatives of any of the reverse transcriptases described above and may be obtained by mutating, for example, the RNase H domain within the reverse transcriptase of interest, for example, by introducing one or more (e.g., one, two, three, four, five, ten, twelve, fifteen, twenty, thirty, etc.) point mutations, one or more (e.g., one, two, three, four, five, ten, twelve, fifteen, twenty, thirty, etc.) deletion mutations, and/or one or more (e.g., one, two, three, four, five, ten, twelve, fifteen, twenty, thirty, etc.) insertion mutations as described elsewhere herein. For example, such mutations are described in U.S. Pat. Nos. 8,541,219 and 8,753,845, and are herein incorporated by reference in their entirety. Accordingly, in some embodiments, RNAseH mutant reverse transcriptases as described herein are envisioned to be utilized.

By an enzyme “substantially reduced in RNase H activity” is meant that the enzyme has reduced RNase H activity as compared to the corresponding wild type or un-mutated reverse trancriptase, or RNase H+ enzyme, such as wild type Maloney Murine Leukemia Virus (M-MLV), Avian Myeloblastosis Virus (AMV) or Rous Sarcoma Virus (RSV) reverse transcriptases. Reverse transcriptases having reduced, substantially reduced, undetectable or lacking RNase H activity have been previously described (see U.S. Pat. Nos. 5,668,005, 6,063,608, and PCT Publication No. WO 98/47912). The RNase H activity of any enzyme may be determined by a variety of assays, such as those described, for example, in U.S. Pat. No. 5,244,797, in Kotewicz, M. L., et al., Nucl. Acids Res. 16:265 (1988), in Gerard, G. F., et al., FOCUS 14(5):91 (1992), in PCT publication number WO 98/47912, and in U.S. Pat. No. 5,668,005, the disclosures of all of which are fully incorporated herein by reference. According to some embodiments, the methods and systems of the disclosure further employs a RNAse inhibitor. According to some embodiments, an RNAse inhibitor is a protein that has RNAse reducing activity. A preferred example of an RNAse inhibitor is ribonuclease/angiogenin inhibitor 1 (RNH1). Exemplary sequence(s) are set forth in SEQ ID No: 16.

According to some embodiments, the present disclosure is also directed, at least in apart, to methods of generating random mutagenesis at a locus of interest. According to some embodiments, the methods and systems of the disclosure are useful for target gene diversification. According to some embodiments, the methods and systems of the disclosure employ a naturally error-prone reverse transcriptase. According to some embodiments, the methods and systems of the disclosure employ a synthetic, more mutagenic reverse transcriptase variant that exhibits reverse transcriptase activity. According to some embodiments, an error-prone reverse transcriptase is a reverse transcriptase from diversity generating retroelements (DGR) within various bacteria and phages. Preferred examples of a genes that encode a functional error-prone reverse transcriptase are Bordetella bacteriophage reverse transcriptase (Brt) gene, Treponema DGR reverse transcriptase gene, Bacteroides DGR reverse transcriptase gene and Eggerthella lenta DGR reverse transcriptase gene. Exemplary sequences are as set forth in SEQ ID Nos: 35-38. According to some embodiments, the methods and systems of the disclosure involve recruitment of an enzyme to the Cas-RT complex with the ability to mutagenize the RNA template, or change the RNA bases to a substrate that the reverse transcriptase is more error-prone in reading. Examples of such an enzyme include ADAR. Examples of the RNA base is 3-methylcytosine.

### Nuclear Localization Signal (NLS)

According to some embodiments, the present invention further comprises one or more nuclear Localization Signals (NLS) or one or more nucleic acid sequences encoding one or more nuclear localization signals. According to some embodiments, the one or more nuclear localization signals are sufficient to drive accumulation of one or more components or subcomponents described herein into the nuclease of a cell. According to some embodiments, the reverse transcriptase as described herein is modified with a nuclear localization signal. According to some embodiments, the reverse transcriptase as described herein is modified to work in eukaryotic cells of interest, such as mammalian cells, by the addition of one or more nuclear localization signals.

### Extended Guide RNA

According to some embodiments, the present invention comprises an extended guide RNA or sequences encoding an extended guide RNA. According to some embodiments, an extended gRNA comprises a gRNA and an RNA template for the reverse transcriptase.

### Guide RNA

According to some embodiments, the present invention comprises a guide RNA or sequence(s) encoding a guide RNA. According to some embodiments, a guide RNA (“gRNA”) is used interchangeabley to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas complex to the target); and (2) a domain that binds a Cas protein. In some embodiments, domain (2) corresponds to a sequence known as a tracrRNA, and comprises a stem-loop structure.

All of the guide RNA may not be synthesized as part of the oligonucleotide. The guide RNA may be considered as comprising a guide head and a guide tail. The guide head is about 15-22 bases in length, about 17-21 bases in length, or about 18-20 bases in length. The guide head is related in sequence to the donor DNA. The guide tail is longer and will generally be invariant in a population of plasmid constructs. The guide tail may be between about 90 and 110 bases, between about 95 and 105 bases, or between about 98 and 100 bases. The guide tail, due to its general invariance, need not be synthesized on the solid array, but can be separately synthesized by any means, including by PCR, solid phase synthesis, or recombinant synthesis. The guide tail can be joined to the oligonucleotide (containing the guide head) separately or at the same time as the oligonucleotide is joined to the plasmid.

Guide nucleic acids may be RNA or DNA molecules. They are selected and coordinated with the nucleic acid-guided sequence-specific nuclease, i.e., the properties of the guide are dictated by the sequence-specific nuclease. Many such sequence-specific nucleases are known. Guide nucleic acids are selected for complementarity to a target site of interest. Desirably the complementarity will be complete within the guide head, but for the desired mutation. Decreased complementarity may lead to loss of specificity and/or efficiency. The guide will be expressed from the plasmid in the case of a guide RNA. To achieve such expression, a suitable promoter will be placed upstream of the guide RNA-coding segment on the carrier plasmid. The transcription promoter may be synthesized as part of the oligonucleotide or may be a part of the plasmid vector. A transcription terminator may optionally be placed downstream from the guide RNA-coding segment. A terminator may prevent read-through transcription of donor nucleic acid. Any terminator functional in mammalian cells, or other desired host cells, known in the art may be used.

According to some embodiments, a guide RNA specifically hybridizes to a target site. The guide RNA forms a complex with a Cas protein described herein and assists in the recognition of the intended cleavage site in the target gene or target gene specific sequence within the host cell's genome by homologous basepairing with the target gene specific sequence. In some embodiments, the guide RNA is provided on a vector, for example, a target selector vector or gene specific vector, encoding a polynucleotide sequence for the guide RNA.

In some embodiments, the guide RNA targets at least one region of the target gene selected from the group consisting of a promoter region, an enhancer region, a repressor region, an insulator region, a silencer region, a region involved in DNA looping with the promoter region, a gene splicing region, or a transcribed region. In certain embodiments, the guide RNA targets a promoter region. In certain embodiments, the guide RNA targets an enhancer region. In certain embodiments, the guide RNA targets a repressor region. In certain embodiments, the guide RNA targets an insulator region. In certain embodiments, the guide RNA targets a silencer region. In certain embodiments, the guide RNA targets a region involved in DNA looping with the promoter region. In certain embodiments, the guide RNA targets a gene splicing region. In certain embodiments, the guide RNA targets a transcribed region.

### RNA Template

According to some embodiments, the extended gRNA comprises a RNA template. The RNA template referred to interchangeably herein as a RNA sequence or the reverse transcriptase template, is the template wherein the reverse transcriptase polymerizes According to some embodiments, the gRNA is extended with the RNA template complementary to the cut site. According to some embodiments, the RNA template is complementary to the cut, non-bound strand. According to some embodiments, the RNA template is constructed to be able to introduce the desired mutations into the target locus.

According to some embodiments the extended gRNA is able to hybridize to the cut non-bound strand. According to some embodiments, the RNA template is able to efficiently complex with the nicked target DNA strand. Once hybridized, a RNA-DNA hybrid is formed. According to some embodiments, the reverse transcriptase primes from the RNA-DNA hybrid, extending the genomic DNA from the site of the nick. According to some embodiments, the reverse transcriptase uses the extended gRNA as a template to introduced desired mutations into the genome. Accordingly, in some embodiments, the RNA template includes one or more mutations to be introduced into the cell of interest.

According to some embodiments, a linker may be operably linked with the RNA template in order to increase the ease with which the RNA template is able to interact with the target strand.

According to some embodiment, the RNA template may be fused to the 5′ end of the gRNA construct or the 3′ end of the gRNA construct. Preferred extended gRNA sequences are as set forth in SEQ ID Nos: 3-6.

According to some embodiments, a DNA product is polymerized. According to some embodiments, the present system and methods described herein further comprises reducing competition from the extended DNA product. According to some embodiments, the extended DNA product may compete with the 5′ end of the native DNA strand. According to some embodiments, one or more DNA repair proteins may help to reduce competition between the extended DNA product and the bound DNA strand. Certain DNA repair proteins may be recruited to cleave the native 5′ bound DNA strand that is competing with the 3′ extended DNA nick.

Examples of DNA repair proteins include 5′ flap endonucleases and 5′ to 3′ exonucleases. Preferred examples 5′flap endonucleases include FEN1, SLX1/SLX4. Exemplary sequence(s) are as set forth in SEQ ID No: 17. Preferred examples 5′ to 3′ exonucleases include but are not limited to TAQ exonuclease domain, T7 exonuclease, Lambda exonuclease, Polymerase A 5′ to 3′ exonuclease domain, exonuclease domain from BST DNA polymerase or BST full polymerase including the exonuclease domain Exemplary sequences are as set forth in SEQ ID Nos: 18-24.

According to some embodiments, the present systems and methods described herein comprise further DNA repair proteins that assist to stabilize and facilitate the extension. DNA repair proteins may further comprise single stranded DNA binding proteins, a helicase, or both. For example, single stranded DNA (ssDNA) binding proteins are recruited to the site of extension to help stabilize the unbound 5′ DNA end and prevent its reannealing. Preferred examples of ssDNA binding proteins include Replication Protein A (RPA), RAD51 ssDNA binding domain, RAD51D ssDNA binding domain, RAD51AP1 ssDNA binding domain, or NEQ199 ssDNA Binding protein. Exemplary sequences are as set forth in SEQ ID Nos: 25-28. A 5′ to 3′ helicase with activity against RNA:DNA hybrids is recruited to help facilitate separation of the 5′ DNA strand from the RNA template. Preferred examples of 5′ to 3′ helicase include PIF1. Exemplary sequence(s) are as set forth in SEQ ID No: 29.

DNA repair proteins may be recruited to the site of extension. According to some embodiments, proteins may be recruited to the site of extension by providing one or more sequences encoding said proteins or proteins thereof as fused on one or more other components or subcomponents of the system as described herein. For example, one or more DNA repair proteins may be provided as fused to the Cas protein. In another example, one or more DNA repair proteins may be provided as fused to the reverse transcriptase. According to some embodiments, proteins may be recruited to the site of extension via secondary recruitment using a two component system. Preferred two component systems comprise MCP-MS2 or Suntag systems, or any other systems similar to those listed herein and as known and practiced in the field.

According to some embodiments, reducing competition from the extended DNA product may comprise introducing two (2) nicks into the non-gRNA target strand. In certain embodiments, 2 nicks in the non-targeted strand disassociates the strand. According to some embodiments, reducing competition from the extended DNA product results in more efficient extension of the 3′ DNA end.

According to some embodiments, the RNA template must be a full length and intact in order to allow the reverse transcriptase to use to introduce the desired mutations into the target locus. In some embodiments, the ends of the RNA template must be produced. For example, the ends of the RNA must be protected from exonucleotic degradation. Accordingly in some embodiments, the extended gRNA comprises further modifications to protect the template from degradation.

For example, in some embodiments, the extended gRNA is modified by comprising further protective sequences. According to some embodiments, the protective sequences protect the template extensions from degradation by endogenous exonucleases, increase the efficiency of targeted genome modification, or both. According to some embodiments, such sequences block 3′ to 5′ or 5′ to 3′ exonuclease activity. Preferred sequences include sequences from Kaposi's sarcoma-associated herpesvirus (KSHV) or from the Flavivirus family, that block 3′ to 5′ or 5′ to 3′ exonuclease activity, respectively.

According to some embodiments, protective sequences block Xrn1 or exosome-mediated degradation of the extended gRNA. For example, a structural viral sequence is added to the 5′ or the 3′ end of the extended gRNA to block either Xrn1 or exosome-mediated degradation of the extended gRNA. According to some embodiments, an exonuclease blocking sequence is used to block degradation of the extended gRNA.

According to some embodiments, the desired mutations are introduced downstream of the nick site by extending from the 3′ nick site. According to some embodiments, the desired mutations are introduced upstream of the nick site. According to some embodiments, desired mutations are introduced upstream by through any method known in the art. For example, using a high fidelity reverse transcriptase with a 3′ to 5′ proofreading activity. Preferably a high fidelity reverse transcriptase comprises a protein that is capable of performing RNA-templated DNA synthesis, has preserved the 3′ to 5′ exonuclease activity, or increases the fidelity with which targeted genomic modification, any combination thereof or all of the foregoing. Preferred examples of a high fidelity reverse transcriptase are DNA polymerase RTX, M160 reverse transcriptase, MMULV reverse transcriptase, MAGMA DNA polymerase, and Foamy virus reverse transcriptase. Exemplary sequences are as set forth in SEQ ID Nos: 30-34.

### Mutations

According to some embodiments, the present invention comprises a mutation introduced into a genome. Any type of mutation that is desirable to build into an oligonucleotide may be used. Mutations may be point mutations, deletion mutations, or insertion mutations, for example. In another example, mutations or modifications described herein may be single nucleotide polymorphism, phosphomimetic mutation, phosphonull mutation, missense mutation, nonsense mutation, synonymous mutation, insertion, deletion, knock-out or knock-in. Inserted nucleic acid within an insertion mutation may be heterologous or native to the host cell.

According to some embodiments, the mutation comprises a deletion of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. According to some embodiments, the mutation comprises a deletion of about 3 base pairs in length. According to some embodiments, the mutation comprises an insertion of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. According to some embodiments, the mutation comprises a point mutation of about 1, 10, 20, 30, 40, 50, 60, 70, 80, 90, 100, 200, 300, 400, 50, 600, 700, 800, 900 or more base pairs, or about 1, 2, 3, 4, 5, 6, 7, 8, 9, 10, 20, 30, 40, 50, 60, 70, 80, 90 or more kb in length, or an entire gene or portion thereof. According to some embodiments, the mutation comprises a point mutation of about 1 base pair in length.

According to some embodiments, desired mutations are introduced downstream of nick site. According to some embodiments, desired mutations are introduced upstream of nick site.

Libraries of Mutations

According to some embodiments, the present invention comprises more than one type of mutation to be introduced into a genome, a collection of more than one type of mutations, or a library of mutations. According to some embodiments, the present invention comprises creating libraries of cells with one or more mutations. The number of different mutations represented in a library may range, for example, from 20, 25, 30, 40, 50, 100, 250, 500, 750, 1,000, 2,000, 5,000, 10,000, 100,000, or 1,000,000 to any of 100, 1,000, 10,000, 100,000, 1,000,000, 10,000,000 or 100,000,000. Ranges with any of these lower and upper limits are contemplated. Different mutations within the library may optionally code for the same amino acids, for example, when looking for optimization of translation. Alternatively, no synonymous mutations may be used within a single library. In some libraries, it may be desirable to make a mutation in every nucleotide or every codon. In other libraries it may be desirable to make all possible mutations in a codon by one or more nucleotide changes. In still other libraries it may be desirable to make mutations in a codon that lead to all possible amino acid changes.

According to some embodiments libraries of cells may be created with one or more mutations or each with a different mutation through performing a low MOI transduction of the gRNA-template construct such that each cell receive at most one.

In some embodiments, the present system and methods further comprise generating random mutations at the locus of interest.

### Constructs

According to some embodiments, the present invention comprises introducing one or more components or subcomponents into a cell of interest. According to some embodiments, the present invention comprises introducing a Cas protein, a reverse transcriptase, and an extended guide RNA comprising a guide RNA and a RNA template into a cell of interest.

According to some embodiments, the one or more components or subcomponents may be introduced into the cell of interest as encoded by one or more genetic constructs. The genetic construct, such as a plasmid, expression cassette or vector, can comprise nucleic acids that encodes the systems, components, or subcomponents described herein, for example, a Cas protein, a reverse transcriptase, and an extended guide RNA comprising a guide RNA and a RNA template. The nucleic acid sequences can make up a genetic construct that can be a vector wherein the vector is capable of expressing the system, components or subcomponents described herein in the cell of interest.

According to some embodiments of the disclosure, the genetic constructs encoding the system, components or subcomponents described herein can be operatively associated or linked with a variety of promoters, terminators and other regulatory elements for expression in various organisms or cells. According to some embodiments, the genetic construct further comprises coding for one or more regulatory elements for genetic expression of one or more coding sequences encoded therein. In some embodiments, the regulatory elements can be a promoter, an enhancer, an initiation codon, a stop codon, or a polyadenylation signal.

Coding sequences can be optimized for stability and high levels of expression. The reading frame of the coding sequences, constructs, vectors, or any combination thereof can be optimized for appropriate expression.

The constructs can also can include one or more nucleotide sequences encoding a selectable marker, which can be used to select a transformed cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence can encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence). Of course, many examples of suitable selectable markers are known in the art and can be used in the constructs described herein.

In some embodiments, the genetic construct encoding the present system, or subcomponents thereof, can be introduced in one construct or in different constructs. In some embodiments, the genetic constructs can be located on a single vector or included on multiple different vectors.

The vector can be a plasmid. The vector can be useful for transfecting cells with nucleic acid encoding the Cas protein, reverse transcriptase, and extended guide RNA comprising a guide RNA and a RNA template described herein, which when the transformed host cell is cultured and maintained under conditions wherein expression of the genetic insert takes place. Plasmids which can be used in the methods described include any that have an origin of replication that is functional in the target cells. These plasmids will typically be linearizable. Often such linearization will be accomplished with a restriction endonuclease that cleaves the plasmid one or a few times only. Other methods, enzymatic or mechanical can be used for linearization. Often the plasmid will have one or more markers that are selectable or easily screenable in an intermediate host cells and/or in the target cells. For example, an antibiotic resistance gene can be used for selecting in a host cell, such as puromycin, blasticidin, or nourothricin. Transcription regulatory elements such as promoters and terminators may also be in the plasmid for controlling transcription of elements of the oligonucleotide.

The genetic constructs disclosed in the present invention may be delivered using any method of DNA delivery to cells, including non-viral and viral methods. Common non-viral delivery methods include transformation and transfection. Non-viral gene delivery can be mediated by physical methods such as electroporation, microinjection, particle-medicated gene transfer (‘gene gun’), impalefection, hydrostatic pressure, continuous infusion, sonication, chemical transfection, lipofection, or DNA injection (DNA vaccination) with and without in vivo electroporation. Viral mediated gene delivery, or viral transduction, utilizes the ability of a virus to inject its DNA inside a host cell. In some embodiments, the genetic constructs intended for delivery are packaged into a replication-deficient viral particle. Common viruses used include retrovirus, lentivirus, adenovirus, adeno-associated virus, and herpes simplex virus.

### Cell of Interest

According to some embodiments, the present invention comprises introducing one or more components or subcomponents into a cell of interest. The cell of interest can be any host that can be transformed with nucleic acids or otherwise made to efficiently take up nucleic acids. For example, a cell of interest may be a prokaryotic cell, a eukaryotic cell, a fungal cell, plant cell, yeast cell, bacterial cell, mammalian cell, or the like. According to some embodiments, the cell is a non-dividing cell. According to some embodiments, the cell of interest is a mammalian cell.

According to some embodiments, the present system and methods can be used with any mammalian cell line, including known cancer lines (for example, hela, MCF7, or K562), primary cells (patient fibroblasts), stem cells (induced pluripotent stem cells and embryonic stem cells), organoids, or any other commonly used cell culture system. In some embodiments, the host cell is selected from the group including, but not limited to, a myoblast, a fibroblast, a glioblastoma, a carcinoma, an epithelial cell, a stem cell. In some embodiments, the host cell is selected from the group including, but not limited to, a HEK cell, a HeLa cell, a vero cell, a BHK cell, a MDCK cell, a NIH 3T3 cell, a Neuro-2a cell, and a CHO cell.

A wide variety of cell lines suitable for use as a host cell include, but are not limited to, C816I, CCRF-CEM, MOLT, mIMCD-3, NHDF, HeLa˜S3, Huh1, Huh4, Huii7, HUVEC, HASMC, HEKn, HEKa, MiaPaCell, Panel, PC-3, TF1, CTLL-2, CIR, Rat6, CV1, RPTE, A10, T24, 0.182, A375, ARH-77, Calul, SW480, SW620, S OV3, S-UT, CaCo2, P388D1, SEM-K2, WEHI-231, HB56, TIB55, Jurkat, J45.0L LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A.?0.780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML TL CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepal cl c7, HL-60, HMEC, HT-29, Jurkat, JY cells, 562 cells, Ku812, KCL22, G 1, KY01, LNCap, Via-ic! 1-48, MC-38, MCF-7, MCF-IOA, MDA-MB-231, MDA-MB-468, MDA-MB-435, MDCK II, MDCK 11, MOR/0.2R, MONO-MAC 6, MTD-1 A, MyEnd, NCI-H69/CPR, NCI-H69/LX10, NCI-H69/LX20, NQ-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vera cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). Preferred examples of useful mammalian cells include human cells, for example, HEK 293T cells.

According to some embodiments, the target locus in the host cell may include EMX1 locus.

Methods of introducing a nucleic acid into a cell of interest are known in the art, and any known method can be used to introduce a nucleic acid (e.g., an expression construct encoding one or more component or subcomponent described herein) into a cell. Suitable methods include, include e.g., viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. According to some embodiments, cells of interest are transformed so that each cell receive at most one gRNA-template construct. For example, cells of interest are transformed at a low multiplicity of infection (MOI).

### EXAMPLES

### Example 1. RNA Templated Genome Editing

### Example 1A) Plasmid Constructs

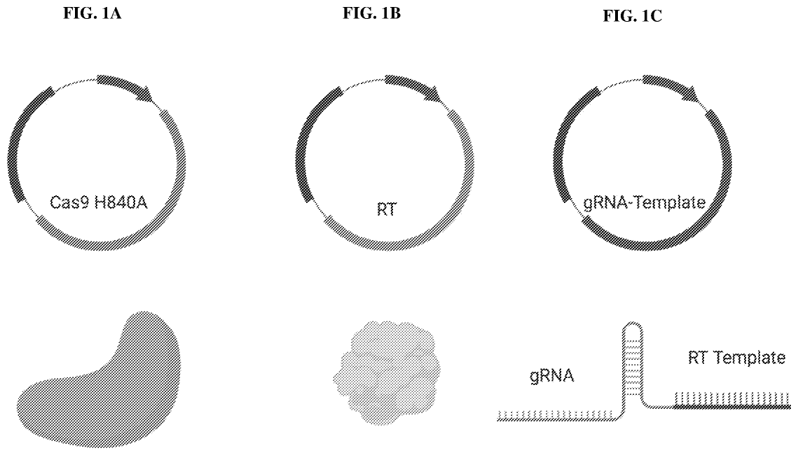

Appropriate constructs were designed or obtained, namely, a plasmid encoding Cas9 H840A nickase (nCas9), a plasmid encoding reverse transcriptase (FIG. 1B), and a plasmid expressing the gRNA-template construct with a sequence encoding the gRNA that targets the locus of interest and the RNA template for reverse transcription which includes the desired mutations, i.e., a sequence complementary to the non-target genomic DNA strand containing the mutation to be introduced (FIG. 1C). A representative schematic is as seen as in FIGS. 1A, 1B, and 1C.

Constructs could be designed or obtained so that the plasmid encoding nCas9 also encodes the RT as fused to the C termini or the N termini.

### Example 1B) Methodology and Molecular Mechanism

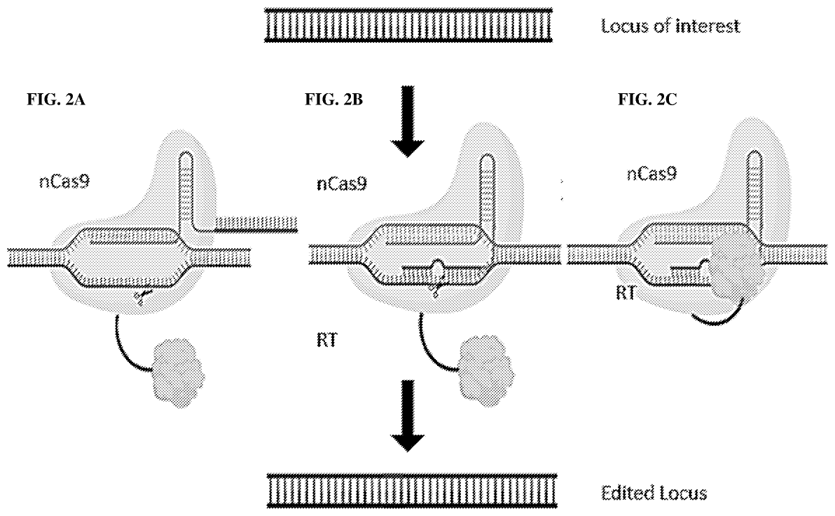

Briefly, host cells were transfected with the plasmids to obtain RNA template genome editing. A representative schematic can be seen in FIGS. 2A, 2B, and 2C.

Once all constructs are within the host cell, the nCas9 complexes with the gRNA-template construct at the genomic locus of interest. After binding to the target locus, the gRNA binds to the target strand and the nCas9 nicks the non-gRNA bound (i.e., the non-target strand). The RNA template hybridizes to the non-target DNA strand, creating a RNA-DNA hybrid. The RT primes from the hybrid by polymerizing from the nick site using the RNA template to introduce mutations in to the target DNA locus.

### Example 2: C-Terminal Vs N-Terminal nCas9-HIV RT Fusions Reverse Transcriptase Activity

The nCas9-RT fusions were tested for reverse-transcription competency. The reverse transcriptase activity level of C-terminal versus N-terminal fused nCas9 were also tested.

Host Cell. HEK293T human cell lines were used as host cells.

Constructs: Appropriate constructs were designed or obtained, namely: a plasmid encoding Cas9 H840A nickase (nCas9) fused with human immunodeficiency virus reverse transcriptase (HIV RT) fused to the C-terminal end of the nCas9; a plasmid encoding Cas9 H840A nickase (nCas9) fused with human immunodeficiency virus reverse transcriptase (HIV RT) fused to the N-terminal end of the nCas9; a plasmid expressing the gRNA-template construct with a sequence encoding the gRNA that targets the locus of interest and a sequence complementary to the non-target genomic DNA strand containing an RNA reporter for HIV RT activity; and a negative control plasmid expressing infrared fluorescent protein (iRFP) instead of RT.

Method. Cells were transfected with the constructs and the amount of single stranded DNA (ssDNA) was qualified via quantitative PCR.

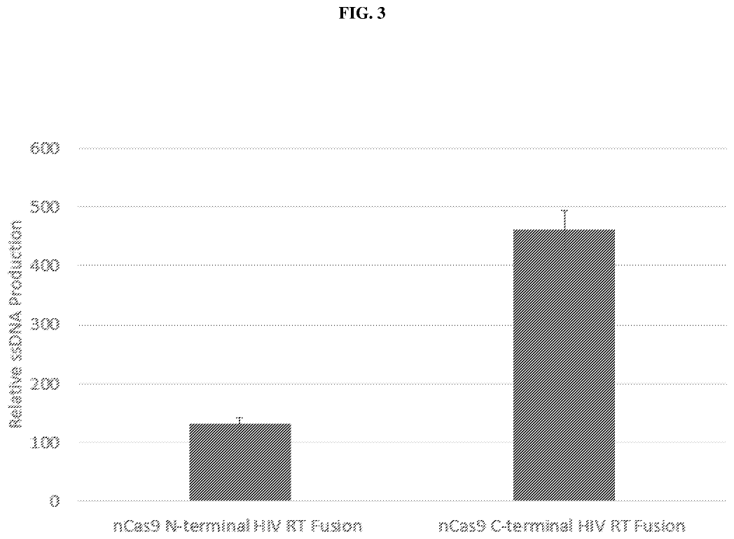

Results. Both N- and C-terminally fused nCas9 demonstrated significant reverse transcriptase activity. C-terminal HIV-RT fusion to nCas9 had approximately three times greater reverse transcriptase activity than the N-terminal fusion. (FIG. 3).

### Example 3: Cas9 RT Fusion Cutting Activity

The C-terminus fused nCas9-RT constructs were tested for nuclease competency, i.e., cutting activity.

Host Cell. HEK293T human cell lines were used as host cells.

Constructs: Appropriate constructs were designed or obtained, namely: a C-terminal fused nCas9 HIV-RT plasmid; a BFP reporter plasmid; and a gRNA against the BFP plasmid.

Method. HEK293T Cells were transfected with the constructs and BFP geometric mean fluorescence intensity measured using flow cytometry.

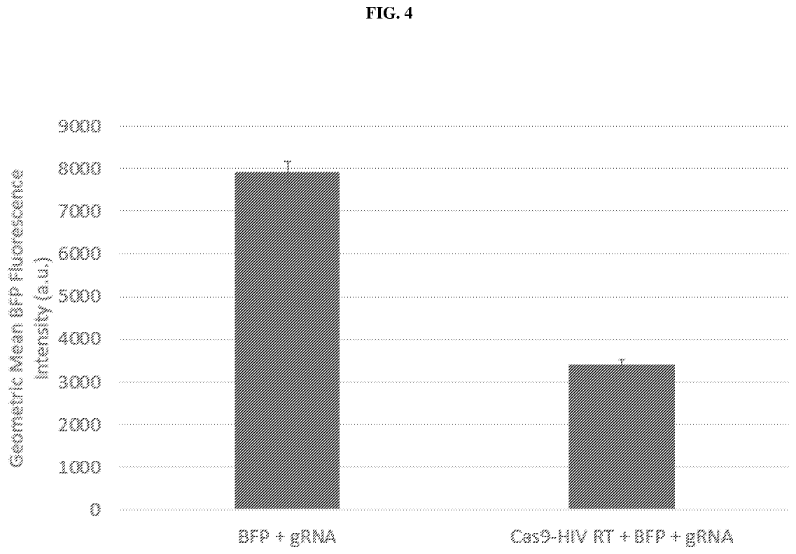

Results. BFP geometric mean fluorescence intensity (a.u.) decreased to 54% in the presence of the nCas9 HIV RT construct, meaning that Cas9 RT fusions still retain nuclease competency. (FIG. 4).

### Example 4: Editing Efficiencies of gRNA-Template Constructs at EMX1 Locus

The activity of the gRNA after being extended with the RNA template complementary to the cut site at the EMX1 locus was tested.

Host Cell. HEK293T human cell lines were used as host cells.

Constructs: Appropriate constructs were designed or obtained, namely: a nuclease competent Cas9 construct, a gRNA construct without a template (“regular gRNA”), a gRNA-template construct with homology to the EMX1 locus seeking to introduce one of three mutations (1 base pair point mutation, or a 3 base pair deletion, or a 3 based pair insertion) (“EMX1 targeting gRNA-template construct”), a gRNA-template construct where the template has no homology to the EMX1 locus (“non-complementary gRNA-template construct”), and a gRNA construct transfected without Cas9 (“gRNA alone”) as a negative control.

Method. HEK293T Cells were transfected with Cas9 and a series of the different extended gRNAs constructs, i.e., Cas9 and regular gRNA, Cas9 and EMX1 targeting gRNA-template construct, Cas9 and non-complementary gRNA-template construct, and with the gRNA alone. Editing efficiencies were measured through next-generation sequencing and the Amplican software package.

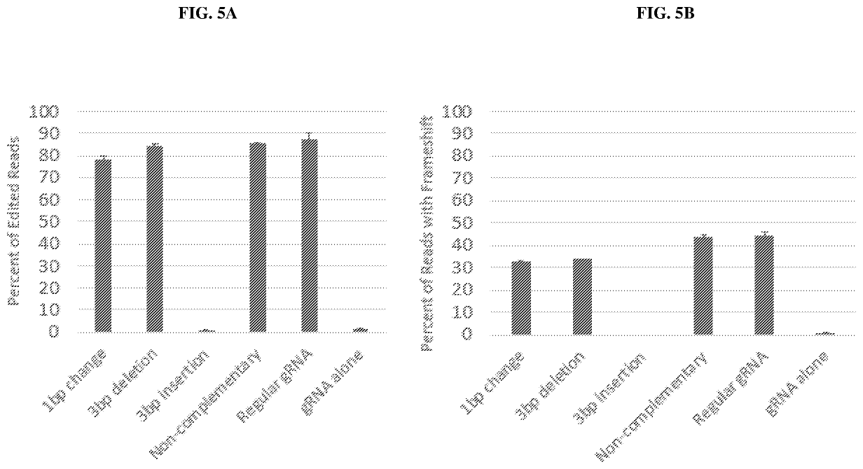

Results. The results indicate that the percentage of edited reads is significantly increased for cells transfected with EMX1 targeting gRNA-template construct as compared to transfection with gRNA alone. (FIG. 5A). The results indicate that the percent of read with frameshift is significantly increased for cells transfected with EMX1 targeting gRNA-template construct as compared to transfection with gRNA alone. (FIG. 5B). Therefore, the results indicate that the RNA template fused to the gRNA is able to efficiently complex with the nicked target DNA strand.

### Example 5: Optimization of RNA Templated Genome Editing

To establish optimization of the system, the following tests may be performed.

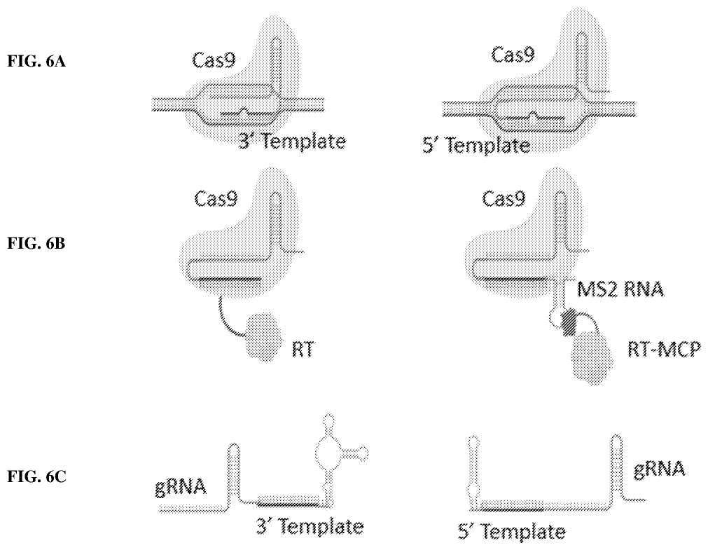

The effect of placing the template region (shown in red) of the gRNA-template construct on the 5′ vs. 3′ end of the construct may be tested. A representative schematic can be seen as in FIG. 6A.

The effect of using a nCas9-HIV RT fusion vs. recruiting HIV RT to the locus via the MCP-MS2 system may be tested. A representative schematic can be seen as in FIG. 6B.

The addition of structured viral sequences to the 5′ or 3′ end of the gRNA-template construct to block either Xrn1 or Exosome-mediated degradation of the gRNA-template may be tested. A representative schematic can be seen as in FIG. 6C.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

SEQUENCE LISTING:

>SEQ ID NO: 1 Cas9 H840A-BPSV40 NLS-GS linker-HIV RT:
ATGGACAAGAAGTACTCCATTGGGCTCGATATCGGCACAAACAGCGTCGGCTGGGCCGT
CATTACGGACGAGTACAAGGTGCCGAGCAAAAAATTCAAAGTTCTGGGCAATACCGATC
GCCACAGCATAAAGAAGAACCTCATTGGCGCCCTCCTGTTCGACTCCGGGGAGACGGCC
GAAGCCACGCGGCTCAAAAGAACAGCACGGCGCAGATATACCCGCAGAAAGAATCGGA
TCTGCTACCTGCAGGAGATCTTTAGTAATGAGATGGCTAAGGTGGATGACTCTTTCTTCC
ATAGGCTGGAGGAGTCCTTTTTGGTGGAGGAGGATAAAAAGCACGAGCGCCACCCAATC
TTTGGCAATATCGTGGACGAGGTGGCGTACCATGAAAAGTACCCAACCATATATCATCTG
AGGAAGAAGCTTGTAGACAGTACTGATAAGGCTGACTTGCGGTTGATCTATCTCGCGCTG
GCGCATATGATCAAATTTCGGGGACACTTCCTCATCGAGGGGGACCTGAACCCAGACAA
CAGCGATGTCGACAAACTCTTTATCCAACTGGTTCAGACTTACAATCAGCTTTTCGAAGA
GAACCCGATCAACGCATCCGGAGTTGACGCCAAAGCAATCCTGAGCGCTAGGCTGTCCA
AATCCCGGCGGCTCGAAAACCTCATCGCACAGCTCCCTGGGGAGAAGAAGAACGGCCTG
TTTGGTAATCTTATCGCCCTGTCACTCGGGCTGACCCCCAACTTTAAATCTAACTTCGACC
TGGCCGAAGATGCCAAGCTTCAACTGAGCAAAGACACCTACGATGATGATCTCGACAAT
CTGCTGGCCCAGATCGGCGACCAGTACGCAGACCTTTTTTTGGCGGCAAAGAACCTGTCA
GACGCCATTCTGCTGAGTGATATTCTGCGAGTGAACACGGAGATCACCAAAGCTCCGCTG
AGCGCTAGTATGATCAAGCGCTATGATGAGCACCACCAAGACTTGACTTTGCTGAAGGC
CCTTGTCAGACAGCAACTGCCTGAGAAGTACAAGGAAATTTTCTTCGATCAGTCTAAAAA
TGGCTACGCCGGATACATTGACGGCGGAGCAAGCCAGGAGGAATTTTACAAATTTATTA
AGCCCATCTTGGAAAAAATGGACGGCACCGAGGAGCTGCTGGTAAAGCTTAACAGAGAA
GATCTGTTGCGCAAACAGCGCACTTTCGACAATGGAAGCATCCCCCACCAGATTCACCTG
GGCGAACTGCACGCTATCCTCAGGCGGCAAGAGGATTTCTACCCCTTTTTGAAAGATAAC
AGGGAAAAGATTGAGAAAATCCTCACATTTCGGATACCCTACTATGTAGGCCCCCTCGCC
CGGGGAAATTCCAGATTCGCGTGGATGACTCGCAAATCAGAAGAGACCATCACTCCCTG
GAACTTCGAGGAAGTCGTGGATAAGGGGGCCTCTGCCCAGTCCTTCATCGAAAGGATGA
CTAACTTTGATAAAAATCTGCCTAACGAAAAGGTGCTTCCTAAACACTCTCTGCTGTACG
AGTACTTCACAGTTTATAACGAGCTCACCAAGGTCAAATACGTCACAGAAGGGATGAGA
AAGCCAGCATTCCTGTCTGGAGAGCAGAAGAAAGCTATCGTGGACCTCCTCTTCAAGAC
GAACCGGAAAGTTACCGTGAAACAGCTCAAAGAAGACTATTTCAAAAAGATTGAATGTT
TCGACTCTGTTGAAATCAGCGGAGTGGAGGATCGCTTCAACGCATCCCTGGGAACGTATC
ACGATCTCCTGAAAATCATTAAAGACAAGGACTTCCTGGACAATGAGGAGAACGAGGAC
ATTCTTGAGGACATTGTCCTCACCCTTACGTTGTTTGAAGATAGGGAGATGATTGAAGAA
CGCTTGAAAACTTACGCTCATCTCTTCGACGACAAAGTCATGAAACAGCTCAAGAGGCG
CCGATATACAGGATGGGGGCGGCTGTCAAGAAAACTGATCAATGGGATCCGAGACAAGC
AGAGTGGAAAGACAATCCTGGATTTTCTTAAGTCCGATGGATTTGCCAACCGGAACTTCA
TGCAGTTGATCCATGATGACTCTCTCACCTTTAAGGAGGACATCCAGAAAGCACAAGTTT
CTGGCCAGGGGGACAGTCTTCACGAGCACATCGCTAATCTTGCAGGTAGCCCAGCTATCA
AAAAGGGAATACTGCAGACCGTTAAGGTCGTGGATGAACTCGTCAAAGTAATGGGAAGG
CATAAGCCCGAGAATATCGTTATCGAGATGGCCCGAGAGAACCAAACTACCCAGAAGGG
ACAGAAGAACAGTAGGGAAAGGATGAAGAGGATTGAAGAGGGTATAAAAGAACTGGGG
TCCCAAATCCTTAAGGAACACCCAGTTGAAAACACCCAGCTTCAGAATGAGAAGCTCTA
CCTGTACTACCTGCAGAACGGCAGGGACATGTACGTGGATCAGGAACTGGACATCAATC
GGCTCTCCGACTACGACGTGGATGCTATCGTGCCCCAGTCTTTTCTCAAAGATGATTCTAT
TGATAATAAAGTGTTGACAAGATCCGATAAAAATAGAGGGAAGAGTGATAACGTCCCCT
CAGAAGAAGTTGTCAAGAAAATGAAAAATTATTGGCGGCAGCTGCTGAACGCCAAACTG
ATCACACAACGGAAGTTCGATAATCTGACTAAGGCTGAACGAGGTGGCCTGTCTGAGTT
GGATAAAGCCGGCTTCATCAAAAGGCAGCTTGTTGAGACACGCCAGATCACCAAGCACG
TGGCCCAAATTCTCGATTCACGCATGAACACCAAGTACGATGAAAATGACAAACTGATT
CGAGAGGTGAAAGTTATTACTCTGAAGTCTAAGCTGGTCTCAGATTTCAGAAAGGACTTT
CAGTTTTATAAGGTGAGAGAGATCAACAATTACCACCATGCGCATGATGCCTACCTGAAT
GCAGTGGTAGGCACTGCACTTATCAAAAAATATCCCAAGCTTGAATCTGAATTTGTTTAC
GGAGACTATAAAGTGTACGATGTTAGGAAAATGATCGCAAAGTCTGAGCAGGAAATAGG
CAAGGCCACCGCTAAGTACTTCTTTTACAGCAATATTATGAATTTTTTCAAGACCGAGAT
TACACTGGCCAATGGAGAGATTCGGAAGCGACCACTTATCGAAACAAACGGAGAAACAG
GAGAAATCGTGTGGGACAAGGGTAGGGATTTCGCGACAGTCCGGAAGGTCCTGTCCATG
CCGCAGGTGAACATCGTTAAAAAGACCGAAGTACAGACCGGAGGCTTCTCCAAGGAAAG
TATCCTCCCGAAAAGGAACAGCGACAAGCTGATCGCACGCAAAAAAGATTGGGACCCCA
AGAAATACGGCGGATTCGATTCTCCTACAGTCGCTTACAGTGTACTGGTTGTGGCCAAAG
TGGAGAAAGGGAAGTCTAAAAAACTCAAAAGCGTCAAGGAACTGCTGGGCATCACAATC
ATGGAGCGATCAAGCTTCGAAAAAAACCCCATCGACTTTCTCGAGGCGAAAGGATATAA
AGAGGTCAAAAAAGACCTCATCATTAAGCTTCCCAAGTACTCTCTCTTTGAGCTTGAAAA
CGGCCGGAAACGAATGCTCGCTAGTGCGGGCGAGCTGCAGAAAGGTAACGAGCTGGCAC
TGCCCTCTAAATACGTTAATTTCTTGTATCTGGCCAGCCACTATGAAAAGCTCAAAGGGT
CTCCCGAAGATAATGAGCAGAAGCAGCTGTTCGTGGAACAACACAAACACTACCTTGAT
GAGATCATCGAGCAAATAAGCGAATTCTCCAAAAGAGTGATCCTCGCCGACGCTAACCT
CGATAAGGTGCTTTCTGCTTACAATAAGCACAGGGATAAGCCCATCAGGGAGCAGGCAG
AAAACATTATCCACTTGTTTACTCTGACCAACTTGGGCGCGCCTGCAGCCTTCAAGTAC
TTCGACACCACCATAGACAGAAAGCGGTACACCTCTACAAAGGAGGTCCTGGACGC
CACACTGATTCATCAGTCAATTACGGGGCTCTATGAAACAAGAATCGACCTCTCTCA
GCTCGGTGGAGACAGCAGGGCTGACCCCAAGAAGAAGAGGAAGGTGGGTTCTGGAA
AACGGACAGCGGACGGTAGCGAGTTTGAGAGTCCGAAGAAAAAGAGGAAAGTAGAGggt
ggttctgccggtggctccggttctggctccagcggtggcagctctggtgcgtccggcacgggtactgcgggtggcactggcagcggttccg
gtactggctctggcCCCATTAGTCCTATTGAGACTGTACCAGTAAAATTAAAGCCAGGAATGGATGGCC
CAAAAGTTAAACAATGGCCATTGACAGAAGAAAAAATAAAAGCATTAGTAGAAATTTGTACAGAA
ATGGAAAAGGAAGGAAAAATTTCAAAAATTGGGCCTGAAAATCCATACAATACTCCAGTATTTGC
CATAAAGAAAAAAGACAGTACTAAATGGAGAAAATTAGTAGATTTCAGAGAACTTAATAAGAGAA
CTCAAGATTTCTGGGAAGTTCAATTAGGAATACCACATCCTGCAGGGTTAAAACAGAAAAAATCA
GTAACAGTACTGGATGTGGGCGATGCATATTTTTCAGTTCCCTTAGATAAAGACTTCAGGAAGTA
TACTGCATTTACCATACCTAGTATAAACAATGAGACACCAGGGATTAGATATCAGTACAATGTGC
TTCCACAGGGATGGAAAGGATCACCAGCAATATTCCAGTGTAGCATGACAAAAATCTTAGAGCC
TTTTAGAAAACAAAATCCAGACATAGTCATCTATCAATACATGGATGATTTGTATGTAGGATCTGA
CTTAGAAATAGGGCAGCATAGAACAAAAATAGAGGAACTGAGACAACATCTGTTGAGGTGGGG
ATTTACCACACCAGACAAAAAACATCAGAAAGAACCTCCATTCCTTTGGATGGGTTATGAACTCC
ATCCTGATAAATGGACAGTACAGCCTATAGTGCTGCCAGAAAAGGACAGCTGGACTGTCAATGA
CATACAGAAATTAGTGGGAAAATTGAATTGGGCAAGTCAGATTTATGCAGGGATTAAAGTAAGG
CAATTATGTAAACITCTTAGGGGAACCAAAGCACTAACAGAAGTAGTACCACTAACAGAAGAAG
CAGAGCTAGAACTGGCAGAAAACAGGGAGATTCTAAAAGAACCGGTACATGGAGTGTATTATGA
CCCATCAAAAGACTTAATAGCAGAAATACAGAAGCAGGGGCAAGGCCAATGGACATATCAAATT
TATCAAGAGCCATTTAAAAATCTGAAAACAGGAAAGTATGCAAGAATGAAGGGTGCCCACACTA
ATGATGTGAAACAATTAACAGAGGCAGTACAAAAAATAGCCACAGAAAGCATAGTAATATGGGG
AAAGACTCCTAAATTTAAATTACCCATACAAAAGGAAACATGGGAAGCATGGTGGACAGAGTATT
GGCAAGCCACCTGGATTCCTGAGTGGGAGTTTGTCAATACCCCTCCCTTAGTGAAGTTATGGTA
CCAGTTAGAGAAAGAACCCATAATAGGAGCAGAAACTTTCTATGTAGATGGGGCAGCCAATAGG
GAAACTAAATTAGGAAAAGCAGGATATGTAACTGACAGAGGAAGACAAAAAGTTGTCCCCCTAA
CGGACACAACAAATCAGAAGACTGAGTTACAAGCAATTCATCTAGCTTTGCAGGATTCGGGATT
AGAAGTAAACATAGTGACAGACTCACAATATGCATTGGGAATCATTCAAGCACAACCAGATAAG
AGTGAATCAGAGTTAGTCAGTCAAATAATAGAGCAGTTAATAAAAAAGGAAAAAGTCTACCTGGC
ATGGGTACCAGCACACAAAGGAATTGGAGGAAATGAACAAGTAGATAAATTGGTCAGTGCTGGA
ATCAGGAAAGTACTAGGCGGGGGTTCTGGGGGAGGATCAGGTGGTGGGTCCGGGGGAGGAA
GCGGGGGTGGCTCTGGGGGTGGATCACCGATTAGCCCGATTGAAACCGTTCCGGTTAAACTG
AAACCGGGTATGGATGGTCCGAAAGTTAAACAGTGGCCTCTGACCGAAGAAAAAATCAAAGCA
CTGGTTGAAATCTGCACCGAGATGGAAAAAGAAGGCAAAATTAGCAAAATCGGTCCGGAAAATC
CGTATAATACACCGGTTTTTGCCATTAAGAAAAAAGATAGCACCAAATGGCGCAAACTGGTGGA
TTTTCGTGAACTGAATAAACGCACCCAGGATTTTTGGGAAGTTCAGCTGGGTATTCCGCATCCG
GCAGGTCTGAAACAGAAAAAAAGCGTTACCGTTCTGGATGTTGGTGATGCATATTTTAGCGTTC
CGCTGGATAAAGATTTCCGTAAATATACCGCATTTACCATCCCGAGCATTAATAACGAAACACCG
GGTATTCGCTATCAGTATAATGTTCTGCCGCAGGGTTGGAAAGGTAGTCCGGCAATTTTTCAGT
GTAGCATGACCAAAATTCTGGAACCGTTTCGTAAACAGAATCCGGATATTGTGATCTACCAGTAT
ATGGATGATCTGTATGTTGGTAGCGATCTGGAAATTGGTCAGCATCGTACCAAAATTGAAGAAC
TGCGTCAGCATCTGCTGCGTTGGGGTTTTACCACACCGGATAAAAAACATCAGAAAGAACCGCC
TTTTCTGTGGATGGGTTATGAACTGCATCCGGATAAATGGACCGTTCAGCCGATTGTTCTGCCG
GAAAAAGATAGCTGGACCGTTAATGATATTCAGAAACTGGTGGGTAAACTGAATTGGGCAAGCC
AGATTTATGCCGGTATTAAAGTTCGTCAGCTGTGTAAACTGCTGCGTGGCACCAAAGCACTGAC
CGAAGTTGTTCCGCTGACAGAAGAAGCAGAACTGGAACTGGCAGAAAATCGTGAAATTCTGAAA
GAACCGGTTCACGGCGTTTATTATGATCCGAGCAAAGATCTGATTGCCGAAATTCAGAAACAGG
GTCAGGGTCAGTGGACCTATCAGATTTATCAAGAACCGTTTAAAAACCTGAAAACCGGCAAATA
TGCACGTATGAAAGGTGCACATACCAACGATGTTAAACAGCTGACCGAAGCAGTTCAGAAAATT
GCAACCGAAAGCATTGTGATTTGGGGTAAAACCCCGAAATTCAAACTGCCGATTCAGAAAGAAA
CCTGGGAAGCATGGTGGACCGAATATTGGCAGGCAACCTGGATTCCGGAATGGGAATTTGTTA
ATACCCCTCCGCTGGTTAAACTGTGGTATCAGCTGGAAAAAGAACCGATTATTGGTGCCGAAAC
CTTTTGA
>SEQ ID NO: 2 HIV RT-GS linker-Cas9 H840A-BPSV40 NLS
ATGCCCATTAGTCCTATTGAGACTGTACCAGTAAAATTAAAGCCAGGAATGGATGGCCCAAAAG
TTAAACAATGGCCATTGACAGAAGAAAAAATAAAAGCATTAGTAGAAATTTGTACAGAAATGGAA
AAGGAAGGAAAAATTTCAAAAATTGGGCCTGAAAATCCATACAATACTCCAGTATTTGCCATAAA
GAAAAAAGACAGTACTAAATGGAGAAAATTAGTAGATTTCAGAGAACTTAATAAGAGAACTCAAG
ATTTCTGGGAAGTTCAATTAGGAATACCACATCCTGCAGGGTTAAAACAGAAAAAATCAGTAACA
GTACTGGATGTGGGCGATGCATATTTTTCAGTTCCCTTAGATAAAGACTTCAGGAAGTATACTGC
ATTTACCATACCTAGTATAAACAATGAGACACCAGGGATTAGATATCAGTACAATGTGCTTCCAC
AGGGATGGAAAGGATCACCAGCAATATTCCAGTGTAGCATGACAAAAATCTTAGAGCCTTTTAG
AAAACAAAATCCAGACATAGTCATCTATCAATACATGGATGATTTGTATGTAGGATCTGACTTAG
AAATAGGGCAGCATAGAACAAAAATAGAGGAACTGAGACAACATCTGTTGAGGTGGGGATTTAC
CACACCAGACAAAAAACATCAGAAAGAACCTCCATTCCTTTGGATGGGTTATGAACTCCATCCT
GATAAATGGACAGTACAGCCTATAGTGCTGCCAGAAAAGGACAGCTGGACTGTCAATGACATAC
AGAAATTAGTGGGAAAATTGAATTGGGCAAGTCAGATTTATGCAGGGATTAAAGTAAGGCAATTA
TGTAAACTTCTTAGGGGAACCAAAGCACTAACAGAAGTAGTACCACTAACAGAAGAAGCAGAGC
TAGAACTGGCAGAAAACAGGGAGATTCTAAAAGAACCGGTACATGGAGTGTATTATGACCCATC
AAAAGACTTAATAGCAGAAATACAGAAGCAGGGGCAAGGCCAATGGACATATCAAATTTATCAA
GAGCCATTTAAAAATCTGAAAACAGGAAAGTATGCAAGAATGAAGGGTGCCCACACTAATGATG
TGAAACAATTAACAGAGGCAGTACAAAAAATAGCCACAGAAAGCATAGTAATATGGGGAAAGAC
TCCTAAATTTAAATTACCCATACAAAAGGAAACATGGGAAGCATGGTGGACAGAGTATTGGCAA
GCCACCTGGATTCCTGAGTGGGAGTTTGTCAATACCCCTCCCTTAGTGAAGTTATGGTACCAGT
TAGAGAAAGAACCCATAATAGGAGCAGAAACTTTCTATGTAGATGGGGCAGCCAATAGGGAAAC
TAAATTAGGAAAAGCAGGATATGTAACTGACAGAGGAAGACAAAAAGTTGTCCCCCTAACGGAC
ACAACAAATCAGAAGACTGAGTTACAAGCAATTCATCTAGCTTTGCAGGATTCGGGATTAGAAGT
AAACATAGTGACAGACTCACAATATGCATTGGGAATCATTCAAGCACAACCAGATAAGAGTGAAT
CAGAGTTAGTCAGTCAAATAATAGAGCAGTTAATAAAAAAGGAAAAAGTCTACCTGGCATGGGT
ACCAGCACACAAAGGAATTGGAGGAAATGAACAAGTAGATAAATTGGTCAGTGCTGGAATCAGG
AAAGTACTAGGCGGGGGTTCTGGGGGAGGATCAGGTGGTGGGTCCGGGGGAGGAAGCGGGG
GTGGCTCTGGGGGTGGATCACCGATTAGCCCGATTGAAACCGTTCCGGTTAAACTGAAACCGG
GTATGGATGGTCCGAAAGTTAAACAGTGGCCTCTGACCGAAGAAAAAATCAAAGCACTGGTTGA
AATCTGCACCGAGATGGAAAAAGAAGGCAAAATTAGCAAAATCGGTCCGGAAAATCCGTATAAT
ACACCGGTTTTTGCCATTAAGAAAAAAGATAGCACCAAATGGCGCAAACTGGTGGATTTTCGTG
AACTGAATAAACGCACCCAGGATTTTTGGGAAGTTCAGCTGGGTATTCCGCATCCGGCAGGTCT
GAAACAGAAAAAAAGCGTTACCGTTCTGGATGTTGGTGATGCATATTTTAGCGTTCCGCTGGAT
AAAGATTTCCGTAAATATACCGCATTTACCATCCCGAGCATTAATAACGAAACACCGGGTATTCG
CTATCAGTATAATGTTCTGCCGCAGGGTTGGAAAGGTAGTCCGGCAATTTTTCAGTGTAGCATG
ACCAAAATTCTGGAACCGTTTCGTAAACAGAATCCGGATATTGTGATCTACCAGTATATGGATGA
TCTGTATGTTGGTAGCGATCTGGAAATTGGTCAGCATCGTACCAAAATTGAAGAACTGCGTCAG
CATCTGCTGCGTTGGGGTTTTACCACACCGGATAAAAAACATCAGAAAGAACCGCCTTTTCTGT
GGATGGGTTATGAACTGCATCCGGATAAATGGACCGTTCAGCCGATTGTTCTGCCGGAAAAAG
ATAGCTGGACCGTTAATGATATTCAGAAACTGGTGGGTAAACTGAATTGGGCAAGCCAGATTTA
TGCCGGTATTAAAGTTCGTCAGCTGTGTAAACTGCTGCGTGGCACCAAAGCACTGACCGAAGTT
GTTCCGCTGACAGAAGAAGCAGAACTGGAACTGGCAGAAAATCGTGAAATTCTGAAAGAACCG
GTTCACGGCGTTTATTATGATCCGAGCAAAGATCTGATTGCCGAAATTCAGAAACAGGGTCAGG
GTCAGTGGACCTATCAGATTTATCAAGAACCGTTTAAAAACCTGAAAACCGGCAAATATGCACGT
ATGAAAGGTGCACATACCAACGATGTTAAACAGCTGACCGAAGCAGTTCAGAAAATTGCAACCG
AAAGCATTGTGATTTGGGGTAAAACCCCGAAATTCAAACTGCCGATTCAGAAAGAAACCTGGGA
AGCATGGTGGACCGAATATTGGCAGGCAACCTGGATTCCGGAATGGGAATTTGTTAATACCCCT
CCGCTGGTTAAACTGTGGTATCAGCTGGAAAAAGAACCGATTATTGGTGCCGAAACCTTTTGAgg
tggttctgccggtggctccggttctggctccagcggtggcagctctggtgcgtccggcacgggtactgcgggtggcactggcagcggttccg
gtactggctctggcGACAAGAAGTACTCCATTGGGCTCGATATCGGCACAAACAGCGTCGGCTG
GGCCGTCATTACGGACGAGTACAAGGTGCCGAGCAAAAAATTCAAAGTTCTGGGCAATA
CCGATCGCCACAGCATAAAGAAGAACCTCATTGGCGCCCTCCTGTTCGACTCCGGGGAG
ACGGCCGAAGCCACGCGGCTCAAAAGAACAGCACGGCGCAGATATACCCGCAGAAAGA
ATCGGATCTGCTACCTGCAGGAGATCTTTAGTAATGAGATGGCTAAGGTGGATGACTCTT
TCTTCCATAGGCTGGAGGAGTCCTTTTTGGTGGAGGAGGATAAAAAGCACGAGCGCCAC
CCAATCTTTGGCAATATCGTGGACGAGGTGGCGTACCATGAAAAGTACCCAACCATATAT
CATCTGAGGAAGAAGCTTGTAGACAGTACTGATAAGGCTGACTTGCGGTTGATCTATCTC
GCGCTGGCGCATATGATCAAATTTCGGGGACACTTCCTCATCGAGGGGGACCTGAACCC
AGACAACAGCGATGTCGACAAACTCTTTATCCAACTGGTTCAGACTTACAATCAGCTTTT
CGAAGAGAACCCGATCAACGCATCCGGAGTTGACGCCAAAGCAATCCTGAGCGCTAGGC
TGTCCAAATCCCGGCGGCTCGAAAACCTCATCGCACAGCTCCCTGGGGAGAAGAAGAAC
GGCCTGTTTGGTAATCTTATCGCCCTGTCACTCGGGCTGACCCCCAACTTTAAATCTAACT
TCGACCTGGCCGAAGATGCCAAGCTTCAACTGAGCAAAGACACCTACGATGATGATCTC
GACAATCTGCTGGCCCAGATCGGCGACCAGTACGCAGACCTTTTTTTGGCGGCAAAGAA
CCTGTCAGACGCCATTCTGCTGAGTGATATTCTGCGAGTGAACACGGAGATCACCAAAGC
TCCGCTGAGCGCTAGTATGATCAAGCGCTATGATGAGCACCACCAAGACTTGACTTTGCT
GAAGGCCCTTGTCAGACAGCAACTGCCTGAGAAGTACAAGGAAATTTTCTTCGATCAGTC
TAAAAATGGCTACGCCGGATACATTGACGGCGGAGCAAGCCAGGAGGAATTTTACAAAT
TTATTAAGCCCATCTTGGAAAAAATGGACGGCACCGAGGAGCTGCTGGTAAAGCTTAAC
AGAGAAGATCTGTTGCGCAAACAGCGCACTTTCGACAATGGAAGCATCCCCCACCAGAT
TCACCTGGGCGAACTGCACGCTATCCTCAGGCGGCAAGAGGATTTCTACCCCTTTTTGAA
AGATAACAGGGAAAAGATTGAGAAAATCCTCACATTTCGGATACCCTACTATGTAGGCC
CCCTCGCCCGGGGAAATTCCAGATTCGCGTGGATGACTCGCAAATCAGAAGAGACCATC
ACTCCCTGGAACTTCGAGGAAGTCGTGGATAAGGGGGCCTCTGCCCAGTCCTTCATCGAA
AGGATGACTAACTTTGATAAAAATCTGCCTAACGAAAAGGTGCTTCCTAAACACTCTCTG
CTGTACGAGTACTTCACAGTTTATAACGAGCTCACCAAGGTCAAATACGTCACAGAAGG
GATGAGAAAGCCAGCATTCCTGTCTGGAGAGCAGAAGAAAGCTATCGTGGACCTCCTCT
TCAAGACGAACCGGAAAGTTACCGTGAAACAGCTCAAAGAAGACTATTTCAAAAAGATT
GAATGTTTCGACTCTGTTGAAATCAGCGGAGTGGAGGATCGCTTCAACGCATCCCTGGGA
ACGTATCACGATCTCCTGAAAATCATTAAAGACAAGGACTTCCTGGACAATGAGGAGAA
CGAGGACATTCTTGAGGACATTGTCCTCACCCTTACGTTGTTTGAAGATAGGGAGATGAT
TGAAGAACGCTTGAAAACTTACGCTCATCTCTTCGACGACAAAGTCATGAAACAGCTCA
AGAGGCGCCGATATACAGGATGGGGGCGGCTGTCAAGAAAACTGATCAATGGGATCCGA
GACAAGCAGAGTGGAAAGACAATCCTGGATTTTCTTAAGTCCGATGGATTTGCCAACCG
GAACTTCATGCAGTTGATCCATGATGACTCTCTCACCTTTAAGGAGGACATCCAGAAAGC
ACAAGTTTCTGGCCAGGGGGACAGTCTTCACGAGCACATCGCTAATCTTGCAGGTAGCCC
AGCTATCAAAAAGGGAATACTGCAGACCGTTAAGGTCGTGGATGAACTCGTCAAAGTAA
TGGGAAGGCATAAGCCCGAGAATATCGTTATCGAGATGGCCCGAGAGAACCAAACTACC
CAGAAGGGACAGAAGAACAGTAGGGAAAGGATGAAGAGGATTGAAGAGGGTATAAAA
GAACTGGGGTCCCAAATCCTTAAGGAACACCCAGTTGAAAACACCCAGCTTCAGAATGA
GAAGCTCTACCTGTACTACCTGCAGAACGGCAGGGACATGTACGTGGATCAGGAACTGG
ACATCAATCGGCTCTCCGACTACGACGTGGATGCTATCGTGCCCCAGTCTTTTCTCAAAG
ATGATTCTATTGATAATAAAGTGTTGACAAGATCCGATAAAAATAGAGGGAAGAGTGAT
AACGTCCCCTCAGAAGAAGTTGTCAAGAAAATGAAAAATTATTGGCGGCAGCTGCTGAA
CGCCAAACTGATCACACAACGGAAGTTCGATAATCTGACTAAGGCTGAACGAGGTGGCC
TGTCTGAGTTGGATAAAGCCGGCTTCATCAAAAGGCAGCTTGTTGAGACACGCCAGATC
ACCAAGCACGTGGCCCAAATTCTCGATTCACGCATGAACACCAAGTACGATGAAAATGA
CAAACTGATTCGAGAGGTGAAAGTTATTACTCTGAAGTCTAAGCTGGTCTCAGATTTCAG
AAAGGACTTTCAGTTTTATAAGGTGAGAGAGATCAACAATTACCACCATGCGCATGATG
CCTACCTGAATGCAGTGGTAGGCACTGCACTTATCAAAAAATATCCCAAGCTTGAATCTG
AATTTGTTTACGGAGACTATAAAGTGTACGATGTTAGGAAAATGATCGCAAAGTCTGAG
CAGGAAATAGGCAAGGCCACCGCTAAGTACTTCTTTTACAGCAATATTATGAATTTTTTC
AAGACCGAGATTACACTGGCCAATGGAGAGATTCGGAAGCGACCACTTATCGAAACAAA
CGGAGAAACAGGAGAAATCGTGTGGGACAAGGGTAGGGATTTCGCGACAGTCCGGAAG
GTCCTGTCCATGCCGCAGGTGAACATCGTTAAAAAGACCGAAGTACAGACCGGAGGCTT
CTCCAAGGAAAGTATCCTCCCGAAAAGGAACAGCGACAAGCTGATCGCACGCAAAAAA
GATTGGGACCCCAAGAAATACGGCGGATTCGATTCTCCTACAGTCGCTTACAGTGTACTG
GTTGTGGCCAAAGTGGAGAAAGGGAAGTCTAAAAAACTCAAAAGCGTCAAGGAACTGCT
GGGCATCACAATCATGGAGCGATCAAGCTTCGAAAAAAACCCCATCGACTTTCTCGAGG
CGAAAGGATATAAAGAGGTCAAAAAAGACCTCATCATTAAGCTTCCCAAGTACTCTCTCT
TTGAGCTTGAAAACGGCCGGAAACGAATGCTCGCTAGTGCGGGCGAGCTGCAGAAAGGT
AACGAGCTGGCACTGCCCTCTAAATACGTTAATTTCTTGTATCTGGCCAGCCACTATGAA
AAGCTCAAAGGGTCTCCCGAAGATAATGAGCAGAAGCAGCTGTTCGTGGAACAACACAA
ACACTACCTTGATGAGATCATCGAGCAAATAAGCGAATTCTCCAAAAGAGTGATCCTCG
CCGACGCTAACCTCGATAAGGTGCTTTCTGCTTACAATAAGCACAGGGATAAGCCCATCA
GGGAGCAGGCAGAAAACATTATCCACTTGTTTACTCTGACCAACTTGGGCGCGCCTGCA
GCCTTCAAGTACTTCGACACCACCATAGACAGAAAGCGGTACACCTCTACAAAGGA
GGTCCTGGACGCCACACTGATTCATCAGTCAATTACGGGGCTCTATGAAACAAGAA
TCGACCTCTCTCAGCTCGGTGGAGACAGCAGGGCTGACCCCAAGAAGAAGAGGAA
GGTGGGTTCTGGAAAACGGACAGCGGACGGTAGCGAGTTTGAGAGTCCGAAGAAAAAG
AGGAAAGTAGATGA
>SEQ ID NO: 3 gRNA-1 base change template
GAGTCCGAGCAGAAGAAGAAgttttagagctagaaatagcaagttaaaataaggctagtccgttatcaacttgaaaaagtggcac
cgagtcggtgccgccaccggttgatgtgatgggagcccTTCcTCTTCTGCTCGGACTCaggcccttcctcc
>SEQ ID NO: 4 gRNA-3 base deletion template
GAGTCCGAGCAGAAGAAGAAgttttagagctagaaatagcaagttaaaataaggctagtccgttatcaacttgaaaaagtggcac
cgagtcggtgccgccaccggttgatgtgatgggagcccTTCTTCTGCTCGGACTCaggcccttcctcc
>SEQ ID NO: 5 gRNA-SPACER-1 base change template
GAGTCCGAGCAGAAGAAGAAgttttagagctagaaatagcaagttaaaataaggctagtccgttatcaacttgaaaaagtggcac
cgagtcggtgcTCTCTCCGCTTATCTTCTCTATTTCCTTTATTCCGTCCCTCCAcgccaccggttgatgtgatgg
gagcccTTCcTCTTCTGCTCGGACTCaggcccttcctcc
>SEQ ID NO: 6 gRNA-SPACER-3 base deletion template
GAGTCCGAGCAGAAGAAGAAgttttagagctagaaatagcaagttaaaataaggctagtccgttatcaacttgaaaaagtggcac
cgagtcggtgcTCTCTCCGCTTATCTTCTCTATTTCCTTTATTCCGTCCCTCCAcgccaccggttgatgtgatgg
gagcccTTCTTCTGCTCGGACTCaggcccttcctcc
>SEQ ID No: 7 PolH:
GCTACTGGACAGGATCGAGTGGTTGCTCTCGTGGACATGGACTGTTTTTTTGTTCAAGTG
GAGCAGCGGCAAAATCCTCATTTGAGGAATAAACCTTGTGCAGTCGTACAGTACAAATC
ATGGAAGGGTGGTGGAATAATTGCAGTGAGTTATGAAGCTCGTGCATTTGGAGTCACTA
GAAGTATGTGGGCAGATGATGCTAAGAAGTTATGTCCAGATCTTCTACTGGCACAAGTTC
GTGAGTCCCGTGGGAAAGCTAACCTCACCAAGTACCGGGAAGCCAGTGTTGAAGTGATG
GAGATAATGTCTCGTTTTGCTGTGATTGAACGTGCCAGCATTGATGAGGCTTACGTAGAT
CTGACCAGTGCCGTACAAGAGAGACTACAAAAGCTACAAGGTCAGCCTATCTCGGCAGA
CTTGTTGCCAAGCACTTACATTGAAGGGTTGCCCCAAGGCCCTACAACGGCAGAAGAGA
CTGTTCAGAAAGAGGGGATGCGAAAACAAGGCTTATTTCAATGGCTCGATTCTCTTCAGA
TTGATAACCTCACCTCTCCAGACCTGCAGCTCACCGTGGGAGCAGTGATTGTGGAGGAAA
TGAGAGCAGCCATAGAGAGGGAGACTGGTTTTCAGTGTTCAGCTGGAATTTCACACAAT
AAGGTCCTGGCAAAACTGGCCTGTGGACTAAACAAGCCCAACCGCCAAACCCTGGTTTC
ACATGGGTCAGTCCCACAGCTCTTCAGCCAAATGCCCATTCGCAAAATCCGTAGTCTTGG
AGGAAAGCTAGGGGCCTCTGTCATTGAGATTCTAGGGATAGAATACATGGGTGAACTGA
CCCAGTTCACTGAATCCCAGCTCCAGAGTCATTTTGGGGAGAAGAATGGGTCTTGGCTAT
ATGCCATGTGCCGAGGGATTGAACATGATCCAGTTAAACCCAGGCAACTACCCAAAACC
ATTGGCTGTAGTAAGAACTTCCCAGGAAAAACAGCTCTTGCTACTCGGGAACAGGTACA
ATGGTGGCTGTTGCAATTAGCCCAGGAACTAGAGGAGAGACTGACTAAAGACCGAAATG
ATAATGACAGGGTAGCCACCCAGCTGGTTGTGAGCATTCGCGTACAAGGAGACAAACGC
CTCAGCAGCCTGCGCCGCTGCTGTGCCCTTACCCGCTATGATGCTCACAAGATGAGCCAT
GATGCATTTACTGTCATCAAGAACTGTAATACTTCTGGAATCCAGACAGAATGGTCTCCT
CCTCTCACAATGCTTTTCCTCTGTGCTACAAAATTTTCTGCCTCTGCCCCTTCATCTTCTAC
AGACATCACCAGCTTCTTGAGCAGTGACCCAAGTTCTCTGCCAAAGGTGCCAGTTACCAG
CTCAGAAGCTAAGACCCAGGGAAGTGGCCCAGCGGTGACAGCCACTAAGAAAGCAACC
ACGTCTCTGGAATCATTCTTCCAAAAAGCTGCAGAAAGGCAGAAAGTTAAAGAAGCTTC
GCTTTCATCTCTTACTGCTCCCACTCAGGCTCCCATGAGCAATTCACCATCCAAGCCCTCA
TTACCTTTTCAAACCAGTCAAAGTACAGGAACTGAGCCCTTCTTTAAGCAGAAAAGTCTG
CTTCTAAAGCAGAAACAGCTTAATAATTCTTCAGTTTCTTCCCCCCAACAAAACCCATGG
TCCAACTGTAAAGCATTACCAAACTCTTTACCAACAGAGTATCCAGGGTGTGTCCCTGTT
TGTGAAGGGGTGTCGAAGCTAGAAGAATCCTCTAAAGCAACTCCTGCAGAGATGGATTT
GGCCCACAACAGCCAAAGCATGCACGCCTCTTCAGCTTCCAAATCTGTGCTGGAGGTGAC
TCAGAAAGCAACCCCAAATCCAAGTCTTCTAGCTGCTGAGGACCAAGTGCCCTGTGAGA
AGTGTGGCTCCCTGGTACCGGTATGGGATATGCCAGAACACATGGACTATCATTTTGCAT
TGGAGTTGCAGAAATCCTTTTTGCAGCCCCACTCTTCAAACCCCCAGGTTGTTTCTGCCGT
ATCTCATCAAGGCAAAAGAAATCCCAAGAGCCCTTTGGCCTGCACTAATAAACGCCCCA
GGCCTGAGGGCATGCAAACATTGGAATCATTTTTTAAGCCATTAACACAT
>SEQ ID No: 8 DinB2:
ACATCCTGGGTCTTGCACGTAGACCTCGATCAATTCCTTGCCAGCGTGGAGTTGCGGCGC
AGACCCGACCTGAGAGGTCTCCCGGTAATCGTAGGGGGATCAGGCGATCCCACCGAGCC
GCGCAAAGTTGTCACGTGTGCTAGTTACGAGGCGCGCGAGTTCGGTGTCCATGCTGGCAT
GCCGCTGAGGGCCGCGGCTCGAAGGTGCCCAGACGCCACATTTCTTCCTTCTGATCCCGC
AGCATACGATGAAGCCAGCGAGCAGGTAATGGGGTTGCTGAGGGACTTGGGGCACCCTT
TGGAAGTATGGGGGTGGGATGAGGCGTACTTGGGTGCCGACTTGGAGCCTGACGCAGAT
CCGGTGGAACTCGCCGAAAGGATAAGAACTGTCGTTGCCGCTGAAACGGGGCTTTCCTG
TTCTGTAGGAATATCCGACAACAAGCAAAGAGCAAAGGTGGCAACTGGGTTTGCAAAAC
CAGCGGGTATCTACGTGCTTACTGAAGCAAATTGGATGACCGTAATGGGCGATAGACCC
CCGGATGCGCTCTGGGGTATCGGGCCTAAAACGACCAAGAAGTTGGCGGCAATGGGCAT
AACAACAGTCGCGGATCTCGCGGCCACCGACGCAAGTGTTCTCACTGCGGCGTTCGGTCC
TAGTACCGGACTGTGGATATTGCTCCTCGCCAAAGGAGGGGGAGATACTGAGGTGTCAA
GTGAGCCGTGGATACCCAGATCCCGCTCACATGTAGTGACTTTTCCGCAGGACCTCACCG
ACCGGCGGGAAATCGATTCCGCCGTCCGCGACCTTGCACTTCAGACACTTACTGAGATCG
TTGAGCAAGGGCGCACCGTTACTAGAGTTGCTGTCACGGTGCGGACATCTACATTTTACA
CGCGAACCAAGATACGAAAGCTGCCAACACCGGGTACTGACGCTGATCAAATAGTGGCG
ACCGCACTGGCAGTCTTGGACCAATTCGAATTGGATCGACCTGTCCGACTCCTTGGCGTT
CGACTCGAGCTTGCAATGGATGATGTTGCGGCACCGACCGTTGGTACCGGGACA
>SEQ ID No: 9 HIV reverse transcriptase:
CCCATTAGTCCTATTGAGACTGTACCAGTAAAATTAAAGCCAGGAATGGATGGCCCAAA
AGTTAAACAATGGCCATTGACAGAAGAAAAAATAAAAGCATTAGTAGAAATTTGcACAG
AAATGGAAAAGGAAGGAAAAATTTCAAAAATTGGGCCTGAAAATCCATACAATACTCCA
GTATTTGCCATAAAGAAAAAAGACAGTACTAAATGGAGAAAATTAGTAGATTTCAGAGA
ACTTAATAAGAGAACTCAAGATTTCTGGGAAGTTCAATTAGGAATACCACATCCTGCAG
GGTTAAAACAGAAAAAATCAGTAACAGTACTGGATGTGGGCGATGCATATTTTTCAGTTC
CCTTAGATAAAGACTTCAGGAAGTATACTGCATTTACCATACCTAGTATAAACAATGAGA
CACCAGGGATTAGATATCAGTACAATGTGCTTCCACAGGGATGGAAAGGATCACCAGCA
ATATTCCAGTGTAGCATGACAAAAATCTTAGAGCCTTTTAGAAAACAAAATCCAGACAT
AGTCATCTATCAATACATGGATGATTTGTATGTAGGATCTGACTTAGAAATAGGGCAGCA
TAGAACAAAAATAGAGGAACTGAGACAACATCTGTTGAGGTGGGGATTTACCACACCAG
ACAAAAAACATCAGAAAGAACCTCCATTCCTTTGGATGGGTTATGAACTCCATCCTGATA
AATGGACAGTACAGCCTATAGTGCTGCCAGAAAAGGACAGCTGGACTGTCAATGACATA
CAGAAATTAGTGGGAAAATTGAATTGGGCAAGTCAGATTTATGCAGGGATTAAAGTAAG
GCAATTATGTAAACTTCTTAGGGGAACCAAAGCACTAACAGAAGTAGTACCACTAACAG
AAGAAGCAGAGCTAGAACTGGCAGAAAACAGGGAGATTCTAAAAGAACCGGTACATGG
AGTGTATTATGACCCATCAAAAGACTTAATAGCAGAAATACAGAAGCAGGGGCAAGGCC
AATGGACATATCAAATTTATCAAGAGCCATTTAAAAATCTGAAAACAGGAAAGTATGCA
AGAATGAAGGGTGCCCACACTAATGATGTGAAACAATTAACAGAGGCAGTACAAAAAAT
AGCCACAGAAAGCATAGTAATATGGGGAAAGACTCCTAAATTTAAATTACCCATACAAA
AGGAAACATGGGAAGCATGGTGGACAGAGTATTGGCAAGCCACCTGGATTCCTGAGTGG
GAGTTTGTCAATACCCCTCCCTTAGTGAAGTTATGGTACCAGTTAGAGAAAGAACCCATA
ATAGGAGCAGAAACTTTCTATGTAGATGGGGCAGCCAATAGGGAAACTAAATTAGGAAA
AGCAGGATATGTAACTGACAGAGGAAGACAAAAAGTTGTCCCCCTAACGGACACAACAA
ATCAGAAGACTGAGTTACAAGCAATTCATCTAGCTTTGCAGGATTCGGGATTAGAAGTA
AACATAGTGACAGACTCACAATATGCATTGGGAATCATTCAAGCACAACCAGATAAGAG
TGAATCAGAGTTAGTCAGTCAAATAATAGAGCAGTTAATAAAAAAGGAAAAAGTCTACC
TGGCATGGGTACCAGCACACAAAGGAATTGGAGGAAATGAACAAGTAGATAAATTGGTC
AGTGCTGGAATCAGGAAAGTACTAGGCGGGGGTTCTGGGGGAGGATCAGGTGGTGGGTC
CGGGGGAGGAAGCGGGGGTGGCTCTGGGGGTGGATCACCGATTAGCCCGATTGAAACCG
TTCCGGTTAAACTGAAACCGGGTATGGATGGTCCGAAAGTTAAACAGTGGCCTCTGACC
GAAGAAAAAATCAAAGCACTGGTTGAAATCTGCACCGAGATGGAAAAAGAAGGCAAAA
TTAGCAAAATCGGTCCGGAAAATCCGTATAATACACCGGTTTTTGCCATTAAGAAAAAA
GATAGCACCAAATGGCGCAAACTGGTGGATTTTCGTGAACTGAATAAACGCACCCAGGA
TTTTTGGGAAGTTCAGCTGGGTATTCCGCATCCGGCAGGTCTGAAACAGAAAAAAAGCG
TTACCGTTCTGGATGTTGGTGATGCATATTTTAGCGTTCCGCTGGATAAAGATTTCCGTAA
ATATACCGCATTTACCATCCCGAGCATTAATAACGAAACACCGGGTATTCGCTATCAGTA
TAATGTTCTGCCGCAGGGTTGGAAAGGTAGTCCGGCAATTTTTCAGTGTAGCATGACCAA
AATTCTGGAACCGTTTCGTAAACAGAATCCGGATATTGTGATCTACCAGTATATGGATGA
TCTGTATGTTGGTAGCGATCTGGAAATTGGTCAGCATCGTACCAAAATTGAAGAACTGCG
TCAGCATCTGCTGCGTTGGGGTTTTACCACACCGGATAAAAAACATCAGAAAGAACCGC
CTTTTCTGTGGATGGGTTATGAACTGCATCCGGATAAATGGACCGTTCAGCCGATTGTTC
TGCCGGAAAAAGATAGCTGGACCGTTAATGATATTCAGAAACTGGTGGGTAAACTGAAT
TGGGCAAGCCAGATTTATGCCGGTATTAAAGTTCGTCAGCTGTGTAAACTGCTGCGTGGC
ACCAAAGCACTGACCGAAGTTGTTCCGCTGACAGAAGAAGCAGAACTGGAACTGGCAGA
AAATCGTGAAATTCTGAAAGAACCGGTTCACGGCGTTTATTATGATCCGAGCAAAGATCT
GATTGCCGAAATTCAGAAACAGGGTCAGGGTCAGTGGACCTATCAGATTTATCAAGAAC
CGTTTAAAAACCTGAAAACCGGCAAATATGCACGTATGAAAGGTGCACATACCAACGAT
GTTAAACAGCTGACCGAAGCAGTTCAGAAAATTGCAACCGAAAGCATTGTGATTTGGGG
TAAAACCCCGAAATTCAAACTGCCGATTCAGAAAGAAACCTGGGAAGCATGGTGGACCG
AATATTGGCAGGCAACCTGGATTCCGGAATGGGAATTTGTTAATACCCCTCCGCTGGTTA
AACTGTGGTATCAGCTGGAAAAAGAACCGATTATTGGTGCCGAAACCTTT
>SEQ ID No: 10 Baboon endogenous virus reverse transcriptase:
ACTGTCTCCCTTCAAGATGAACACAGACTGTTTGACATCCCTGTTACTACATCCCTCCCTG
ACGTATGGTTGCAGGATTTCCCTCAAGCGTGGGCCGAGACAGGTGGTCTTGGTCGGGCA
AAATGTCAGGCTCCAATAATCATTGATCTGAAGCCCACAGCCGTTCCGGTTAGTATAAAA
CAGTACCCAATGAGTCTCGAGGCACATATGGGGATTCGACAACACATTATAAAATTTCTG
GAATTGGGGGTCTTGAGACCGTGTCGCAGTCCTTGGAACACGCCCTTGCTGCCGGTCAAG
AAACCTGGTACCCAGGATTACCGCCCGGTGCAAGATCTTCGCGAAATAAATAAGCGCAC
TGTTGACATCCATCCAACTGTCCCCAATCCATACAATCTGCTTTCCACATTGAAGCCGGA
TTATAGCTGGTACACCGTCCTGGACCTTAAGGATGCCTTCTTTTGTCTCCCTCTCGCTCCA
CAGTCCCAGGAGCTTTTTGCGTTCGAGTGGAAGGACCCCGAGCGAGGGATTTCTGGGCA
GTTGACGTGGACCCGCCTGCCGCAGGGATTTAAGAACAGCCCCACACTCTTTGATGAAGC
CCTCCACAGAGACCTGACTGATTTCCGAACGCAGCATCCGGAGGTGACACTGCTGCAAT
ATGTGGATGATCTCCTCCTTGCTGCGCCAACTAAAAAAGCGTGCACGCAGGGTACGAGA
CATCTCTTGCAGGAGCTTGGAGAGAAAGGCTATAGGGCGAGCGCCAAAAAAGCTCAAAT
CTGCCAGACGAAGGTCACCTACCTTGGATACATATTGTCCGAAGGGAAGAGGTGGCTCA
CTCCCGGGAGGATAGAAACAGTAGCTCGCATTCCTCCGCCCCGCAATCCAAGGGAGGTG
AGAGAATTCCTTGGGACAGCTGGTTTTTGTCGATTGTGGATCCCCGGCTTTGCCGAGTTG
GCCGCTCCGCTGTATGCGCTTACAAAAGAGAGCACGCCCTTCACCTGGCAAACTGAACAT
CAGCTCGCCTTTGAAGCGCTTAAAAAAGCACTGCTCTCCGCACCGGCGTTGGGCCTGCCG
GACACGTCCAAACCTTTCACTCTCTTCCTGGACGAGCGGCAAGGAATAGCTAAAGGAGT
GCTGACCCAGAAACTTGGGCCATGGAAGAGGCCTGTCGCATATCTGTCTAAGAAGCTCG
ATCCCGTTGCAGCGGGATGGCCCCCATGCCTGCGGATAATGGCGGCAACAGCTATGCTTG
TAAAGGACAGCGCAAAACTTACTTTGGGGCAACCACTGACAGTCATAACTCCTCATACA
CTTGAAGCGATCGTGCGACAACCACCAGACCGCTGGATTACAAATGCTAGACTCACCCA
TTACCAGGCTCTGTTGTTGGACACAGACAGAGTGCAATTTGGTCCGCCCGTCACCCTTAA
TCCTGCTACCCTCCTTCCGGTGCCAGAAAATCAACCCTCCCCACACGATTGCCGACAGGT
TCTCGCTGAGACACACGGGACCCGCGAAGACCTGAAAGATCAGGAACTGCCTGATGCCG
ATCATACGTGGTACACAGATGGGAGCAGTTACCTGGATTCAGGAACAAGAAGGGCAGGA
GCCGCAGTCGTGGACGGTCATAATACGATCTGGGCCCAGTCATTGCCCCCTGGGACTAGC
GCCCAGAAGGCGGAGCTCATTGCTCTGACCAAAGCGTTGGAACTTTCCAAGGGTAAGAA
AGCTAACATTTACACGGACAGTCGCTATGCTTTTGCTACTGCTCACACCCATGGAAGTAT
ATACGAGCGGCGAGGACTGTTGACTTCAGAGGGTAAAGAAATCAAAAATAAGGCCGAA
ATAATTGCGCTCTTGAAGGCTCTGTTCCTGCCGCAAGAAGTGGCTATCATCCATTGTCCA
GGTCATCAGAAGGGGCAAGACCCGGTCGCAGTTGGTAACCGGCAAGCAGATAGAGTAGC
GAGACAAGCCGCAATGGCAGAAGTTCTGACCTTGGCGACTGAACCCGACAACACTTCAC
ATATAACT
>SEQ ID No: 11 Woolly monkey reverse transcriptase:
GTGTTGAACCTCGAGGAGGAATATCGACTCCATGAAAAGCCCGTTCCGTCCAGTATTGAC
CCCTCCTGGCTCCAACTGTTTCCTACAGTATGGGCAGAGCGAGCGGGGATGGGCCTGGCT
AATCAAGTCCCGCCAGTTGTTGTTGAGCTCCGCTCTGGAGCATCTCCGGTAGCGGTCCGA
CAGTACCCAATGAGTAAGGAAGCTCGGGAGGGGATCCGCCCCCACATTCAACGCTTTCT
GGATCTGGGCGTACTCGTACCTTGCCAGTCACCATGGAATACACCGCTCCTGCCAGTAAA
AAAGCCTGGCACAAATGACTATAGACCTGTGCAGGACCTGAGGGAGATCAACAAACGGG
TGCAAGACATACATCCTACAGTCCCTAACCCCTACAACTTGCTGAGCAGCCTTCCGCCCA
GTCACACATGGTACTCTGTCCTGGACCTTAAAGACGCTTTTTTTTGTTTGAAGTTGCATCC
AAATTCTCAACCCTTGTTCGCATTCGAGTGGAGGGACCCAGAAAAGGGAAACACAGGCC
AGCTGACCTGGACTAGACTGCCCCAAGGATTCAAAAACAGCCCAACGTTGTTCGATGAA
GCTTTGCACAGAGATCTCGCACCGTTCCGAGCTCTCAATCCTCAAGTCGTACTGCTGCAG
TACGTAGACGATCTTTTGGTAGCTGCGCCGACTTATCGGGATTGTAAAGAAGGCACTCAG
AAGCTCCTTCAAGAACTGTCAAAACTCGGCTATAGGGTCTCAGCTAAAAAAGCTCAGCT
GTGCCAGAAAGAGGTCACATATCTCGGTTACTTGCTTAAGGAAGGGAAGCGATGGCTTA
CGCCGGCCCGAAAAGCGACCGTTATGAAGATACCCCCTCCGACTACGCCCCGCCAAGTC
CGGGAGTTCCTGGGAACAGCCGGTTTCTGCCGGCTTTGGATTCCCGGATTCGCTAGTTTG
GCTGCGCCCCTGTATCCCCTCACGAAAGAATCTATTCCTTTTATTTGGACTGAGGAACAC
CAAAAGGCCTTTGATAGAATAAAAGAAGCCTTGTTGTCAGCGCCCGCACTGGCCCTGCCT
GACCTGACGAAACCATTTACACTCTACGTCGATGAGCGCGCTGGTGTGGCACGGGGAGT
ACTGACTCAAACGCTCGGTCCATGGCGCCGACCAGTCGCGTACCTCTCTAAGAAACTTGA
TCCAGTCGCATCAGGATGGCCGACATGCCTTAAAGCAGTAGCTGCCGTTGCCCTGCTCTT
GAAGGACGCAGACAAACTCACACTCGGCCAGAATGTGACAGTCATCGCGAGTCACTCCC
TGGAGTCCATCGTAAGACAACCTCCAGACCGCTGGATGACAAACGCACGCATGACACAT
TACCAATCTCTGCTTCTGAATGAGCGGGTCAGCTTTGCGCCGCCCGCTGTACTTAATCCC
GCGACCCTTCTTCCTGTGGAAAGTGAGGCGACACCCGTTCACAGGTGCTCAGAGATTCTT
GCTGAAGAAACAGGCACCCGGAGAGACCTTAAAGATCAACCCCTGCCGGGTGTTCCGGC
GTGGTATACCGACGGTAGCAGTTTCATTGCGGAAGGGAAGCGACGAGCCGGCGCTGCGA
TCGTTGATGGGAAGAGGACTGTGTGGGCTTCCTCCCTGCCTGAAGGGACATCTGCTCAAA
AGGCTGAGCTCGTCGCCCTTACACAAGCCCTTCGATTGGCGGAAGGCAAGGACATAAAC
ATCTATACAGATTCCCGGTATGCCTTTGCTACTGCACATATACATGGTGCAATTTACAAA
CAGAGGGGCCTCTTGACAAGTGCTGGTAAGGATATCAAAAACAAGGAGGAAATCCTGGC
GTTGTTGGAGGCAATTCACCTCCCAAAGCGCGTTGCAATAATCCATTGTCCGGGTCACCA
AAAAGGCAACGACCCAGTGGCGACAGGGAACAGACGGGCTGACGAGGCAGCGAAGCAA
GCTGCGCTGTCCACCCGCGTGTTGGCAGAGACAACAAAACCG
>SEQ ID No: 12 Avian reticuloendotheliosis virus reverse transcriptase:
GTGTTGAACCTCGAGGAGGAATATCGACTCCATGAAAAGCCCGTTCCGTCCAGTATTGAC
CCCTCCTGGCTCCAACTGTTTCCTACAGTATGGGCAGAGCGAGCGGGGATGGGCCTGGCT
AATCAAGTCCCGCCAGTTGTTGTTGAGCTCCGCTCTGGAGCATCTCCGGTAGCGGTCCGA
CAGTACCCAATGAGTAAGGAAGCTCGGGAGGGGATCCGCCCCCACATTCAACGCTTTCT
GGATCTGGGCGTACTCGTACCTTGCCAGTCACCATGGAATACACCGCTCCTGCCAGTAAA
AAAGCCTGGCACAAATGACTATAGACCTGTGCAGGACCTGAGGGAGATCAACAAACGGG
TGCAAGACATACATCCTACAGTCCCTAACCCCTACAACTTGCTGAGCAGCCTTCCGCCCA
GTCACACATGGTACTCTGTCCTGGACCTTAAAGACGCTTTTTTTTGTTTGAAGTTGCATCC
AAATTCTCAACCCTTGTTCGCATTCGAGTGGAGGGACCCAGAAAAGGGAAACACAGGCC
AGCTGACCTGGACTAGACTGCCCCAAGGATTCAAAAACAGCCCAACGTTGTTCGATGAA
GCTTTGCACAGAGATCTCGCACCGTTCCGAGCTCTCAATCCTCAAGTCGTACTGCTGCAG
TACGTAGACGATCTTTTGGTAGCTGCGCCGACTTATCGGGATTGTAAAGAAGGCACTCAG
AAGCTCCTTCAAGAACTGTCAAAACTCGGCTATAGGGTCTCAGCTAAAAAAGCTCAGCT
GTGCCAGAAAGAGGTCACATATCTCGGTTACTTGCTTAAGGAAGGGAAGCGATGGCTTA
CGCCGGCCCGAAAAGCGACCGTTATGAAGATACCCCCTCCGACTACGCCCCGCCAAGTC
CGGGAGTTCCTGGGAACAGCCGGTTTCTGCCGGCTTTGGATTCCCGGATTCGCTAGTTTG
GCTGCGCCCCTGTATCCCCTCACGAAAGAATCTATTCCTTTTATTTGGACTGAGGAACAC
CAAAAGGCCTTTGATAGAATAAAAGAAGCCTTGTTGTCAGCGCCCGCACTGGCCCTGCCT
GACCTGACGAAACCATTTACACTCTACGTCGATGAGCGCGCTGGTGTGGCACGGGGAGT
ACTGACTCAAACGCTCGGTCCATGGCGCCGACCAGTCGCGTACCTCTCTAAGAAACTTGA
TCCAGTCGCATCAGGATGGCCGACATGCCTTAAAGCAGTAGCTGCCGTTGCCCTGCTCTT
GAAGGACGCAGACAAACTCACACTCGGCCAGAATGTGACAGTCATCGCGAGTCACTCCC
TGGAGTCCATCGTAAGACAACCTCCAGACCGCTGGATGACAAACGCACGCATGACACAT
TACCAATCTCTGCTTCTGAATGAGCGGGTCAGCTTTGCGCCGCCCGCTGTACTTAATCCC
GCGACCCTTCTTCCTGTGGAAAGTGAGGCGACACCCGTTCACAGGTGCTCAGAGATTCTT
GCTGAAGAAACAGGCACCCGGAGAGACCTTAAAGATCAACCCCTGCCGGGTGTTCCGGC
GTGGTATACCGACGGTAGCAGTTTCATTGCGGAAGGGAAGCGACGAGCCGGCGCTGCGA
TCGTTGATGGGAAGAGGACTGTGTGGGCTTCCTCCCTGCCTGAAGGGACATCTGCTCAAA
AGGCTGAGCTCGTCGCCCTTACACAAGCCCTTCGATTGGCGGAAGGCAAGGACATAAAC
ATCTATACAGATTCCCGGTATGCCTTTGCTACTGCACATATACATGGTGCAATTTACAAA
CAGAGGGGCCTCTTGACAAGTGCTGGTAAGGATATCAAAAACAAGGAGGAAATCCTGGC
GTTGTTGGAGGCAATTCACCTCCCAAAGCGCGTTGCAATAATCCATTGTCCGGGTCACCA
AAAAGGCAACGACCCAGTGGCGACAGGGAACAGACGGGCTGACGAGGCAGCGAAGCAA
GCTGCGCTGTCCACCCGCGTGTTGGCAGAGACAACAAAACCG
>SEQ ID No: 13 Feline endogenous virus reverse transcriptase:
CTCCAAGATTTTCCGCAAGCTTGGGCCGAAACTGGCGGCTTGGGACGAGCGAAGTGCCA
GGTTCCGATTATTATTGACCTTAAACCTACAGCAATGCCTGTTTCCATTAGGCAGTATCCA
ATGAGCAAAGAGGCACATATGGGAATTCAACCACATATTACCCGGTTCCTGGAGCTGGG
GGTTTTGCGGCCATGCCGATCACCATGGAATACTCCACTGCTTCCTGTTAAGAAGCCCGG
TACCCGCGACTACCGCCCAGTGCAGGATCTTAGGGAAGTGAACAAAAGGACTATGGATA
TTCACCCAACCGTTCCCAACCCATATAATCTGCTGAGCACACTCTCTCCCGACCGAACCT
GGTATACAGTTCTCGATTTGAAAGATGCGTTCTTTTGCCTGCCTTTGGCTCCTCAGAGCCA
AGAACTCTTTGCGTTTGAGTGGCGCGATCCGGAACGCGGTATCTCAGGGCAGTTGACCTG
GACACGCCTTCCTCAGGGTTTTAAAAATAGCCCAACGCTTTTCGATGAAGCGTTGCATCG
GGATCTTACAGATTTCAGGACACAGCATCCCGAGGTTACATTGCTGCAGTATGTGGATGA
TCTGCTTCTGGCTGCTCCGACGAAGGAGGCCTGTATTAGAGGTACTAAACACCTTCTGCG
AGAGCTTGGCGATAAAGGTTATAGGGCCTCTGCGAAAAAAGCGCAGATCTGTCAAACAA
AGGTCACGTATTTGGGATATATTTTGAGTGAAGGTAAACGATGGCTCACCCCGGGGCGG
ATTGAGACTGTCGCACACATACCACCTCCACAAAATCCTCGGGAAGTCCGCGAGTTCCTC
GGCACCGCGGGATTCTGTAGACTTTGGATCCCGGGATTCGCTGAACTTGCGGCACCCCTC
TACGCGCTCACCAAGGAATCTGCTCCTTTCACGTGGCAGGAGAAGCACCAGTCCGCGTTC
GAGGCCCTTAAGGAAGCTTTGCTTTCTGCACCAGCCCTGGGCCTGCCCGATACGAGTAAA
CCCTTTACTCTCTTTATAGATGAGAAGCAGGGGATTGCGAAAGGCGTGCTGACACAAAA
GCTCGGGCCGTGGAAACGCCCGGTCGCCTACTTGTCTAAGAAGCTTGACCCAGTCGCTGC
AGGATGGCCACCCTGCCTGAGGATCATGGCGGCCACTGCTATGCTCGTCAAGGATTCAGC
AAAGCTCACGCTGGGTCAGCCTTTGACGGTAATTACTCCGCATGCACTTGAGGCAATTGT
TCGGCAAACTCCTGATAGATGGATCACGAATGCTCGCCTTACGCATTACCAAGCACTCCT
GCTTGATACCGATAGGATTCAATTTGGACCACCTGTCACTCTTAACCCTGCGACTCTGCTT
CCGGCGCCAGAGGATCAACAAAGCGCTCACGACTGTAGGCAGGTACTTGCTGAAACCCA
TGGAACTCGAGAGGACCTTAAGGATCAAGAGCTCCCCGACGCAGACCATAGCTGGTACA
CAGACGGGTCCAGTTACATAGACTCTGGCACACGCAGAGCAGGGGCTGCTGTGGTGGAC
GGTCATCACATTATATGGGCCCAGTCACTTCCCCCGGGGACATCAGCCCAAAAGGCGGA
GCTCATAGCATTGACAAAAGCTTTGGAACTGAGTGAAGGTAAAAAAGCTAACATTTACA
CGGACTCACGGTATGCCTTCGCCACGGCGCACACGCACGGCTCCATATACGAGCGGCGA
GGATTGCTCACATCTGAGGGAAAGGAAATAAAGAATAAGGCCGAAATAATAGCCCTGTT
GAAAGCTTTGTTTCTCCCTCGCAAAGTTGCGATTATCCATTGCCCAGGCCATCAGAAAGG
ACAAGACCCTATCGCTACTGGGAATAGACAGGCCGATCAGGTTGCCAGACAGGTTGCCG
TGGCTGAAACTCTTACACTCACGACGAAGCTT
>SEQ ID No: 14 Gibbon leukemia virus reverse transcriptase:
GTTTTGAACCTCGAAGAAGAGTACCGGCTGCACGAAAAACCGGTCCCTTCAAGCATCGA
CCCTTCTTGGCTTCAGCTCTTCCCGACCGTTTGGGCAGAAAGAGCTGGTATGGGCCTCGC
GAACCAGGTACCTCCCGTAGTGGTGGAGTTGAGGAGCGGTGCGTCCCCCGTAGCTGTGA
GGCAGTATCCTATGTCTAAAGAAGCGCGCGAAGGTATACGCCCCCATATCCAAAAGTTTC
TGGACCTGGGTGTCCTCGTTCCATGTCGCTCCCCGTGGAATACCCCTTTGCTGCCGGTAA
AGAAGCCTGGAACTAATGATTACCGCCCCGTCCAAGATCTTCGAGAGATTAATAAACGC
GTACAGGATATCCACCCAACTGTACCAAATCCCTACAATCTCCTGAGCAGTCTTCCTCCT
TCATACACGTGGTATTCAGTGCTCGATCTTAAAGATGCCTTCTTTTGCCTGAGACTTCATC
CTAATAGTCAACCGCTCTTTGCTTTTGAATGGAAAGATCCAGAAAAAGGCAACACTGGTC
AGCTGACGTGGACGAGGCTTCCTCAGGGTTTTAAAAATTCCCCCACCCTCTTCGATGAGG
CGCTTCATCGAGACCTCGCTCCTTTCAGAGCTCTGAATCCCCAAGTGGTACTGCTTCAGT
ACGTCGATGATCTGTTGGTTGCCGCTCCGACTTATGAGGACTGCAAGAAGGGCACACAG
AAGCTCCTGCAGGAACTTAGCAAACTTGGCTACAGAGTGTCTGCGAAGAAAGCTCAATT
GTGTCAGAGAGAGGTTACATATCTGGGCTACCTTTTGAAAGAGGGAAAAAGATGGCTGA
CACCAGCCAGGAAGGCAACAGTAATGAAGATTCCTGTACCCACTACGCCCCGGCAAGTA
AGAGAATTTTTGGGTACCGCAGGATTTTGCAGACTGTGGATCCCTGGCTTTGCGTCACTT
GCCGCACCCCTTTACCCACTTACTAAGGAATCCATCCCTTTTATCTGGACTGAGGAGCAC
CAGCAGGCCTTTGACCACATCAAAAAAGCACTGCTGAGTGCGCCAGCTTTGGCCCTGCCT
GACCTGACGAAGCCATTTACGTTaTACATCGACGAGAGGGCTGGTGTGGCACGGGGGGT
GCTCACGCAAACGCTCGGCCCTTGGAGGCGGCCAGTTGCTTACCTTAGTAAGAAGCTTGA
CCCAGTTGCGTCAGGCTGGCCGACATGCTTGAAAGCCGTTGCCGCGGTCGCCCTGTTGTT
GAAGGACGCTGACAAGTTGACGCTGGGGCAAAATGTCACTGTGATTGCGTCCCACTCTCT
CGAGAGTATCGTTCGCCAACCCCCCGACAGGTGGATGACTAACGCCAGAATGACACACT
ACCAGTCACTTCTCTTGAACGAAAGGGTTAGCTTCGCCCCACCCGCCGTCCTGAATCCGG
CGACTCTTCTTCCTGTGGAAAGTGAGGCCACACCAGTACATAGATGCTCAGAGATACTTG
CCGAAGAAACAGGAACCCGGAGGGACCTGGAAGATCAACCTTTGCCGGGCGTACCAACC
TGGTATACAGACGGATCTTCCTTTATTACGGAAGGCAAGCGACGGGCGGGTGCTCCTATC
GTTGATGGGAAGCGGACAGTATGGGCGAGCAGCCTTCCAGAAGGCACTTCTGCTCAGAA
AGCGGAGTTGGTTGCACTCACTCAAGCGCTTAGACTTGCTGAGGGGAAGAATATTAATAT
ATATACGGATTCTCGCTATGCATTCGCGACGGCCCACATCCATGGCGCAATCTACAAGCA
GCGCGGATTGCTGACCTCCGCTGGCAAGGATATAAAGAATAAGGAGGAGATTCTGGCGC
TGCTTGAGGCGATACATTTGCCACGCAGGGTAGCCATAATACATTGCCCCGGACACCAG
AGGGGCTCTAATCCGGTGGCCACTGGCAACCGAAGAGCGGACGAGGCCGCTAAGCAAGC
AGCACTTTCAACGCGGGTACTTGCCGGTACGACCAAACCC
>SEQ ID No: 15 Walleye dermal sarcoma virus reverse transcriptase:
TCCTGCCAGACGAAGAATACATTGAACATCGACGAGTATTTGCTGCAATTTCCGGACCAA
CTTTGGGCCTCCCTTCCTACTGACATTGGCAGGATGCTTGTACCTCCAATTACCATAAAA
ATAAAGGACAACGCGAGCCTTCCGTCTATTCGACAATACCCATTGCCCAAGGATAAAAC
CGAGGGCCTCAGGCCGCTCATTAGTTCCCTCGAAAATCAGGGGATCCTTATAAAATGCCA
TTCTCCGTGTAATACACCAATCTTCCCTATCAAGAAGGCTGGGCGCGATGAATATAGAAT
GATACACGACCTGCGCGCTATTAATAATATAGTGGCTCCACTGACTGCTGTTGTCGCGTC
CCCCACCACAGTGCTTAGCAACCTCGCCCCTAGCCTGCATTGGTTCACAGTCATTGACCT
TAGTAATGCATTTTTTAGCGTACCTATACACAAGGACAGTCAATACTTGTTTGCCTTCACT
TTCGAGGGGCACCAATACACTTGGACCGTCCTTCCCCAGGGTTTCATTCATAGTCCCACG
CTCTTTTCTCAAGCTCTTTACCAGTCACTCCATAAGATCAAGTTTAAAATCTCTAGCGAAA
TTTGCATTTACATGGATGACGTACTCATAGCCTCAAAAGACAGGGACACGAATCTTAAAG
ATACAGCGGTTATGCTTCAGCATCTGGCATCCGAGGGGCACAAGGTGTCCAAAAAGAAA
TTGCAGTTGTGTCAGCAAGAGGTTGTGTACCTTGGACAACTCCTGACCCCTGAAGGTCGG
AAAATTCTTCCAGATCGAAAGGTTACAGTCAGCCAATTCCAGCAACCTACTACGATCCGA
CAAATTCGGGCGTTTCTTGGACTCGTGGGTTATTGTAGACATTGGATCCCAGAGTTCTCC
ATACACTCCAAATTCCTGGAGAAGCAGTTGAAGAAGGACACGGCGGAGCCGTTTCAATT
GGACGATCAGCAGGTTGAAGCATTCAACAAACTTAAACATGCGATAACCACCGCGCCAG
TTCTTGTGGTACCAGATCCTGCCAAGCCCTTTCAGTTaTACACGAGTCACAGCGAGCACG
CATCTATTGCCGTTTTGACGCAAAAGCATGCAGGAAGAACAAGGCCAATTGCCTTTCTTT
CCTCTAAGTTCGATGCTATCGAGTCAGGCCTTCCCCCGTGTCTGAAGGCTTGCGCCAGTA
TTCACCGCTCCTTGACCCAGGCTGACTCCTTCATACTGGGCGCACCCCTGATTATCTACAC
AACTCACGCTATCTGCACACTCCTCCAGAGGGACCGAAGCCAGCTTGTAACCGCATCTCG
ATTTAGCAAGTGGGAAGCCGATCTTCTTAGACCGGAATTGACATTTGTGGCTTGCTCCGC
GGTGAGCCCCGCGCACCTaTACATGCAATCCTGTGAAAATAATATTCCACCGCATGACTG
CGTTCTCCTCACCCACACAATCTCAAGGCCGCGGCCGGACTTGAGTGATCTGCCAATTCC
GGACCCGGACATGACCCTGTTCAGCGATGGATCTTATACCACCGGACGGGGGGGTGCAG
CAGTAGTCATGCATCGCCCCGTTACGGATGATTTCATCATAATCCACCAACAGCCGGGTG
GAGCCTCCGCGCAAACAGCGGAACTCCTCGCTCTCGCCGCGGCGTGCCATCTTGCCACGG
ACAAAACAGTCAACATATACACTGACTCACGGTACGCGTATGGCGTCGTTCACGATTTTG
GTCACCTCTGGATGCACAGGGGATTCGTAACTAGTGCCGGTACGCCGATAAAAAATCAT
AAGGAGATAGAATATCTTCTCAAGCAAATTATGAAGCCCAAGCAGGTATCCGTTATAAA
AATTGAAGCACACACCAAAGGCGTAAGCATGGAGGTTCGGGGCAATGCAGCTGCAGATG
AGGCGGCTAAAAACGCTGTGTTTTTGGTACAGCGG
>SEQ ID No: 16 RNH1:
AGCCTGGACATCCAGAGCCTGGACATCCAGTGTGAGGAGCTGAGCGACGCTAGATGGGC
CGAGCTCCTCCCTCTGCTCCAGCAGTGCCAAGTGGTCAGGCTGGACGACTGTGGCCTCAC
GGAAGCACGGTGCAAGGACATCAGCTCTGCACTTCGAGTCAACCCTGCACTGGCAGAGC
TCAACCTGCGCAGCAACGAGCTGGGCGATGTCGGCGTGCATTGCGTGCTCCAGGGCCTG
CAGACCCCCTCCTGCAAGATCCAGAAGCTGAGCCTCCAGAACTGCTGCCTGACGGGGGC
CGGCTGCGGGGTCCTGTCCAGCACACTACGCACCCTGCCCACCCTGCAGGAGCTGCACCT
CAGCGACAACCTCTTGGGGGATGCGGGCCTGCAGCTGCTCTGCGAAGGACTCCTGGACC
CCCAGTGCCGCCTGGAAAAGCTGCAGCTGGAGTATTGCAGCCTCTCGGCTGCCAGCTGCG
AGCCCCTGGCCTCCGTGCTCAGGGCCAAGCCGGACTTCAAGGAGCTCACGGTTAGCAAC
AACGACATCAATGAGGCTGGCGTTCATGTGCTATGCCAGGGCCTGAAGGACTCCCCCTGC
CAGCTGGAGGCGCTCAAGCTGGAGAGCTGCGGTGTGACATCAGACAACTGCCGGGACCT
GTGCGGCATTGTGGCCTCCAAGGCCTCGCTGCGGGAGCTGGCCCTGGGCAGCAACAAGC
TGGGTGATGTGGGCATGGCGGAGCTGTGCCCAGGGCTGCTCCACCCCAGCTCCAGGCTC
AGGACCCTGTGGATCTGGGAGTGTGGCATCACTGCCAAGGGCTGCGGGGATCTGTGCCG
TGTCCTCAGGGCCAAGGAGAGCCTGAAGGAGCTCAGCCTGGCCGGCAACGAGCTGGGGG
ATGAGGGTGCCCGACTGTTGTGTGAGACCCTGCTGGAACCTGGCTGCCAGCTGGAGTCGC
TGTGGGTGAAGTCCTGCAGCTTCACAGCCGCCTGCTGCTCCCACTTCAGCTCAGTGCTGG
CCCAGAACAGGTTTCTCCTGGAGCTACAGATAAGCAACAACAGGCTGGAGGATGCGGGC
GTGCGGGAGCTGTGCCAGGGCCTGGGCCAGCCTGGCTCTGTGCTGCGGGTGCTCTGGTTG
GCCGACTGCGATGTGAGTGACAGCAGCTGCAGCAGCCTCGCCGCAACCCTGTTGGCCAA
CCACAGCCTGCGTGAGCTGGACCTCAGCAACAACTGCCTGGGGGACGCGGGCATCCTGC
AGCTGGTGGAGAGCGTCCGGCAGCCGGGCTGCCTCCTGGAGCAGCTGGTCCTGTACGAC
ATTTACTGGTCTGAGGAGATGGAGGACCGGCTGCAGGCCCTGGAGAAGGACAAGCCATC
CCTGAGGGTCATCTCC
>SEQ ID No: 17 FEN1:
GGAATTCAAGGCCTGGCCAAACTAATTGCTGATGTGGCCCCCAGTGCCATCCGGGAGAA
TGACATCAAGAGCTACTTTGGCCGTAAGGTGGCCATTGATGCCTCTATGAGCATTTATCA
GTTCCTGATTGCTGTTCGCCAGGGTGGGGATGTGCTGCAGAATGAGGAGGGTGAGACCA
CCAGCCACCTGATGGGCATGTTCTACCGCACCATTCGCATGATGGAGAACGGCATCAAG
CCCGTGTATGTCTTTGATGGCAAGCCGCCACAGCTCAAGTCAGGCGAGCTGGCCAAACG
CAGTGAGCGGCGGGCTGAGGCAGAGAAGCAGCTGCAGCAGGCTCAGGCTGCTGGGGCC
GAGCAGGAGGTGGAAAAATTCACTAAGCGGCTGGTGAAGGTCACTAAGCAGCACAATG
ATGAGTGCAAACATCTGCTGAGCCTCATGGGCATCCCTTATCTTGATGCACCCAGTGAGG
CAGAGGCCAGCTGTGCTGCCCTGGTGAAGGCTGGCAAAGTCTATGCTGCGGCTACCGAG
GACATGGACTGCCTCACCTTCGGCAGCCCTGTGCTAATGCGACACCTGACTGCCAGTGAA
GCCAAAAAGCTGCCAATCCAGGAATTCCACCTGAGCCGGATTCTGCAGGAGCTGGGCCT
GAACCAGGAACAGTTTGTGGATCTGTGCATCCTGCTAGGCAGTGACTACTGTGAGAGTAT
CCGGGGTATTGGGCCCAAGCGGGCTGTGGACCTCATCCAGAAGCACAAGAGCATCGAGG
AGATCGTGCGGCGACTTGACCCCAACAAGTACCCTGTGCCAGAAAATTGGCTCCACAAG
GAGGCTCACCAGCTCTTCTTGGAACCTGAGGTGCTGGACCCAGAGTCTGTGGAGCTGAA
GTGGAGCGAGCCAAATGAAGAAGAGCTGATCAAGTTCATGTGTGGTGAAAAGCAGTTCT
CTGAGGAGCGAATCCGCAGTGGGGTCAAGAGGCTGAGTAAGAGCCGCCAAGGCAGCAC
CCAGGGCCGCCTGGATGATTTCTTCAAGGTGACCGGCTCACTCTCTTCAGCTAAGCGCAA
GGAGCCAGAACCCAAGGGATCCACTAAGAAGAAGGCAAAGACTGGGGCAGCAGGGAAG
TTTAAAAGGGGAAAA
>SEQ ID No: 18 TAQ exonuclease domain
CGCGGAATGCTCCCACTCTTCGAACCTAAGGGCAGAGTTCTTCTTGTTGACGGACACCAC
TTGGCATATAGAACATTCCATGCACTCAAAGGGCTCACGACCTCACGGGGAGAACCTGT
GCAAGCTGTGTACGGTTTTGCCAAGAGTTTGTTGAAGGCCCTCAAGGAGGATGGTGATGC
TGTAATAGTTGTATTTGATGCCAAGGCTCCTTCTTTCCGACATGAGGCTTATGGCGGCTAT
AAGGCTGGGCGGGCGCCTACACCAGAAGATTTTCCTCGACAACTGGCGTTGATCAAAGA
GTTGGTTGATTTGCTCGGACTCGCCCGACTTGAGGTTCCGGGATACGAAGCCGACGACGT
GTTGGCATCTTTGGCAAAGAAGGCGGAAAAAGAAGGATACGAGGTACGGATTCTTACAG
CTGACAAGGATCTGTACCAGTTGTTGTCAGATCGCATACACGTTTTGCATCCCGAGGGTT
ACCTTATTACACCCGCCTGGCTCTGGGAGAAATACGGCCTTCGGCCCGACCAATGGGCTG
ATTATCGAGCCCTGACGGGTGACGAATCAGATAACCTGCCCGGCGTTAAAGGGATTGGT
GAGAAAACGGCCCGAAAGTTGCTTGAAGAATGGGGCTCTTTGGAGGCACTTCTCAAGAA
CCTGGACCGCCTGAAACCTGCCATCCGCGAAAAAATACTCGCACACATGGATGATCTCA
AACTCAGCTGGGACTTGGCGAAAGTCCGAACAGATCTGCCTCTCGAAGTGGACTTTGCA
AAGAGGCGGGAGCCAGACAGGGAACGACTCAGGGCCTTCCTGGAACGACTGGAATTTGG
ATCATTGTTGCACGAGTTCGGACTCCTGGAATCTGGTGGTGGAGGTTCTGGTGGTGGTGG
CAGC
>SEQ ID No: 19 T7 exonuclease
GCACTTCTTGACCTTAAACAATTCTATGAGTTACGTGAAGGCTGCGACGACAAGGGTATC
CTTGTGATGGACGGCGACTGGCTGGTCTTCCAAGCTATGAGTGCTGCTGAGTTTGATGCC
TCTTGGGAGGAAGAGATTTGGCACCGATGCTGTGACCACGCTAAGGCCCGTCAGATTCTT
GAGGATTCCATTAAGTCCTACGAGACCCGTAAGAAGGCTTGGGCAGGTGCTCCAATTGTC
CTTGCGTTCACCGATAGTGTTAACTGGCGTAAAGAACTGGTTGACCCGAACTATAAGGCT
AACCGTAAGGCCGTGAAGAAACCTGTAGGGTACTTTGAGTTCCTTGATGCTCTCTTTGAG
CGCGAAGAGTTCTATTGCATCCGTGAGCCTATGCTTGAGGGTGATGACGTTATGGGAGTT
ATTGCTTCCAATCCGTCTGCCTTCGGTGCTCGTAAGGCTGTAATCATCTCTTGCGATAAGG
ACTTTAAGACCATCCCTAACTGTGACTTCCTGTGGTGTACCACTGGTAACATCCTGACTC
AGACCGAAGAGTCCGCTGACTGGTGGCACCTCTTCCAGACCATCAAGGGTGACATCACT
GATGGTTACTCAGGGATTGCTGGATGGGGTGATACCGCCGAGGACTTCTTGAATAACCCG
TTCATAACCGAGCCTAAAACGTCTGTGCTTAAGTCCGGTAAGAACAAAGGCCAAGAGGT
TACTAAATGGGTTAAACGCGACCCTGAGCCTCATGAGACGCTTTGGGACTGCATTAAGTC
CATTGGCGCGAAGGCTGGTATGACCGAAGAGGATATTATCAAGCAGGGCCAAATGGCTC
GAATCCTACGGTTCAACGAGTACAACTTTATTGACAAGGAGATTTACCTGTGGAGACCG
>SEQ ID No: 20 Lambda exonuclease
acaccggacattatcctgcagcgtaccgggatcgatgtgagagctgtcgaacagggggatgatgcgtggcacaaattacggctcggcgtcatc
accgcttcagaagttcacaacgtgatagcaaaaccccgctccggaaagaagtggcctgacatgaaaatgtcctacttccacaccctgcttgct
gaggtttgcaccggtgtggctccggaagttaacgctaaagcactggcctggggaaaacagtacgagaacgacgccagaaccctgtttgaattc
acttccggcgtgaatgttactgaatccccgatcatctatcgcgacgaaagtatgcgtaccgcctgctctcccgatggtttatgcagtgacggc
aacggccttgaactgaaatgcccgtttacctcccgggatttcatgaagttccggctcggtggtttcgaggccataaagtcagcttacatggcc
caggtgcagtacagcatgtgggtgacgcgaaaaaatgcctggtactttgccaactatgacccgcgtatgaagcgtgaaggcctgcattatgtc
gtgattgagcgggatgaaaagtacatggcgagttttgacgagatcgtgccggagttcatcgaaaaaatggacgaggcactggctgaaattgg
ttttgtatttggggagcaatggcga
>SEQ ID No: 21 Polymerase A 5′ to 3′ exonuclease domain (5′ to 3′ exonuclease domain from
E. coli DNA polymerase)
GTTCAGATCCCCCAAAATCCACTTATCCTTGTAGATGGTTCATCTTATCTTTATCGCGCAT
ATCACGCGTTTCCCCCGCTGACTAACAGCGCAGGCGAGCCGACCGGTGCGATGTATGGT
GTCCTCAACATGCTGCGCAGTCTGATCATGCAATATAAACCGACGCATGCAGCGGTGGTC
TTTGACGCCAAGGGAAAAACCTTTCGTGATGAACTGTTTGAACATTACAAATCACATCGC
CCGCCAATGCCGGACGATCTGCGTGCACAAATCGAACCCTTGCACGCGATGGTTAAAGC
GATGGGACTGCCGCTGCTGGCGGTTTCTGGCGTAGAAGCGGACGACGTTATCGGTACTCT
GGCGCGCGAAGCCGAAAAAGCCGGGCGTCCGGTGCTGATCAGCACTGGCGATAAAGATA
TGGCGCAGCTGGTGACGCCAAATATTACGCTTATCAATACCATGACGAATACCATCCTCG
GACCGGAAGAGGTGGTGAATAAGTACGGCGTGCCGCCAGAACTGATCATCGATTTCCTG
GCGCTGATGGGTGACTCCTCTGATAACATTCCTGGCGTACCGGGCGTCGGTGAAAAAACC
GCGCAGGCATTGCTGCAAGGTCTTGGCGGACTGGATACGCTGTATGCCGAGCCAGAAAA
AATTGCTGGGTTGAGCTTCCGTGGCGCGAAAACAATGGCAGCGAAGCTCGAGCAAAACA
AAGAAGTTGCTTATCTCTCATACCAGCTGGCGACGATTAAAACCGACGTTGAACTGGAGC
TGACCTGTGAACAACTGGAAGTGCAGCAACCGGCAGCGGAAGAGTTGTTGGGGCTGTTC
AAAAAGTATGAGTTCAAACGCTGGACTGCTGATGTCGAAGCGGGCAAATGGTTACAGGC
CAAAGGGGCAAAACCAGCCGCGAAGCCACAGGAAACCAGTGTTGCAGACGAAGCACCA
GAAGTGACGGCAACG
>SEQ ID No: 22 5′ to 3′ exonuclease domain from BST DNA polymerase
AAGAAGAAATTGGTTCTGATCGACGGAAACTCCGTTGCGTATAGAGCGTTCTTCGCGCTC
CCTCTCTTGCATAACGACAAGGGTATCCACACGAACGCGGTCTACGGGTTCACTATGATG
CTTAACAAAATCCTGGCTGAGGAGCAACCAACTCACCTCCTCGTCGCATTTGATGCTGGG
AAAACAACCTTCCGGCACGAAACATTCCAGGAATATAAAGGCGGAAGGCAACAGACGC
CGCCAGAACTGTCAGAGCAATTTCCTCTGCTTCGAGAGCTCCTTAAAGCTTATAGGATAC
CGGCATACGAGCTCGATCACTACGAGGCGGACGATATTATCGGAACGCTTGCTGCTCGA
GCAGAGCAGGAGGGCTTCGAGGTCAAGATTATCTCCGGGGACCGAGACTTGACTCAACT
TGCTTCACGCCATGTAACAGTCGACATAACGAAAAAAGGGATTACAGATATTGAACCCT
ATACACCAGAGACGGTACGCGAAAAGTACGGCCTCACCCCAGAGCAGATAGTTGATCTC
AAAGGTCTCATGGGCGACAAGTCAGACAACATCCCAGGTGTCCCAGGGATTGGGGAAAA
AACAGCTGTCAAACTTTTGAAACAGTTCGGTACAGTGGAAAACGTTCTTGCGTCCATAGA
CGAAGTAAAAGGTGAGAAGCTCAAAGAGAATCTTAGGCAACATAGAGACTTGGCATTGT
TGTCTAAACAACTCGCGAGTATATGTCGAGATGCGCCTGTAGAGCTTTCCCTTGACGATA
TTGTGTACGAGGGACAGGACCGGGAAAAGGTGATTGCTCTTTTCAAAGAACTCGGATTC
CAGTCTTTTCTTGAGAAAATGGCTGCCCCC
>SEQ ID No: 23 BST DNA polymerase without exonuclease domain:
GCGGCTGAGGGTGAGAAGCCTCTTGAGGAGATGGAGTTTGCGATAGTCGACGTTATTAC
TGAGGAAATGCTCGCTGATAAAGCCGCGCTCGTTGTTGAGGTAATGGAAGAGAACTATC
ATGACGCCCCCATCGTCGGTATAGCGCTGGTAAACGAACATGGGCGATTTTTCATGCGGC
CCGAAACAGCGTTGGCAGACAGTCAATTTCTTGCCTGGCTTGCAGACGAGACGAAGAAA
AAAAGCATGTTTGACGCGAAACGCGCGGTAGTGGCACTCAAATGGAAGGGCATCGAGCT
CAGGGGTGTAGCCTTCGATCTCCTGCTCGCTGCGTACCTTCTTAATCCCGCGCAGGATGC
AGGCGACATAGCCGCTGTCGCAAAGATGAAGCAATATGAGGCGGTCCGATCCGATGAAG
CCGTTTACGGCAAGGGCGTGAAACGGAGTCTCCCTGATGAGCAAACACTTGCGGAACAT
CTTGTGCGAAAAGCCGCAGCGATATGGGCTCTGGAACAGCCATTTATGGATGACTTGCG
AAACAACGAGCAAGATCAGCTGTTGACGAAGTTGGAACAACCGCTTGCGGCGATACTGG
CGGAGATGGAATTCACGGGGGTGAACGTTGATACGAAAAGGCTTGAGCAGATGGGATCA
GAACTCGCTGAACAACTTAGAGCCATCGAACAAAGAATATACGAACTTGCGGGGCAGGA
ATTCAATATAAATAGCCCAAAACAACTTGGGGTCATACTCTTTGAGAAGCTTCAACTCCC
CGTATTGAAAAAGACGAAGACGGGGTATAGTACAAGTGCGGATGTCCTGGAAAAGTTGG
CGCCGCATCACGAAATTGTAGAAAATATACTGCATTACAGGCAACTTGGGAAACTCCAA
TCAACGTACATAGAAGGACTCCTTAAAGTTGTCCGACCTGATACAGGCAAGGTCCACAC
GATGTTTAATCAAGCACTTACGCAAACCGGTCGCCTGAGCTCTGCGGAGCCAAATCTCCA
GAATATACCGATTCGGCTGGAAGAAGGTCGCAAAATTCGGCAGGCGTTCGTACCTAGCG
AACCTGATTGGCTTATATTCGCGGCGGATTACTCTCAGATAGAGCTTAGGGTATTGGCTC
ACATTGCCGATGACGACAACTTGATTGAAGCGTTCCAGCGCGATTTGGACATACATACTA
AGACAGCAATGGATATCTTCCACGTGTCTGAGGAGGAGGTAACTGCTAACATGCGGCGG
CAGGCAAAGGCCGTAAACTTTGGTATTGTTTATGGAATAAGCGACTACGGGCTCGCCCA
GAACCTTAACATCACACGCAAAGAAGCCGCCGAGTTTATTGAGAGATATTTCGCAAGTTT
CCCCGGAGTAAAACAATACATGGAGAATATCGTACAAGAGGCTAAGCAGAAGGGCTATG
TCACCACATTGCTCCACAGAAGACGGTATTTGCCAGACATTACTAGTCGAAACTTTAACG
TGAGGTCATTCGCAGAGCGGACGGCGATGAATACACCCATTCAAGGAAGTGCAGCTGAC
ATTATCAAAAAGGCCATGATTGACCTCGCAGCTAGGTTGAAAGAAGAACAGCTCCAGGC
CCGCCTGCTGCTCCAGGTGCATGATGAGCTCATACTCGAAGCCCCGAAGGAGGAAATAG
AACGGCTGTGCGAGTTGGTCCCAGAAGTAATGGAGCAAGCTGTCACGCTCCGAGTTCCC
CTTAAGGTGGACTACCATTATGGTCCAACGTGGTATGATGCTAAG
>SEQ ID No: 24 BST full polymerase with exonuclease domain:
AAGAAGAAATTGGTTCTGATCGACGGAAACTCCGTTGCGTATAGAGCGTTCTTCGCGCTC
CCTCTCTTGCATAACGACAAGGGTATCCACACGAACGCGGTCTACGGGTTCACTATGATG
CTTAACAAAATCCTGGCTGAGGAGCAACCAACTCACCTCCTCGTCGCATTTGATGCTGGG
AAAACAACCTTCCGGCACGAAACATTCCAGGAATATAAAGGCGGAAGGCAACAGACGC
CGCCAGAACTGTCAGAGCAATTTCCTCTGCTTCGAGAGCTCCTTAAAGCTTATAGGATAC
CGGCATACGAGCTCGATCACTACGAGGCGGACGATATTATCGGAACGCTTGCTGCTCGA
GCAGAGCAGGAGGGCTTCGAGGTCAAGATTATCTCCGGGGACCGAGACTTGACTCAACT
TGCTTCACGCCATGTAACAGTCGACATAACGAAAAAAGGGATTACAGATATTGAACCCT
ATACACCAGAGACGGTACGCGAAAAGTACGGCCTCACCCCAGAGCAGATAGTTGATCTC
AAAGGTCTCATGGGCGACAAGTCAGACAACATCCCAGGTGTCCCAGGGATTGGGGAAAA
AACAGCTGTCAAACTTTTGAAACAGTTCGGTACAGTGGAAAACGTTCTTGCGTCCATAGA
CGAAGTAAAAGGTGAGAAGCTCAAAGAGAATCTTAGGCAACATAGAGACTTGGCATTGT
TGTCTAAACAACTCGCGAGTATATGTCGAGATGCGCCTGTAGAGCTTTCCCTTGACGATA
TTGTGTACGAGGGACAGGACCGGGAAAAGGTGATTGCTCTTTTCAAAGAACTCGGATTC
CAGTCTTTTCTTGAGAAAATGGCTGCCCCCGCGGCTGAGGGTGAGAAGCCTCTTGAGGAG
ATGGAGTTTGCGATAGTCGACGTTATTACTGAGGAAATGCTCGCTGATAAAGCCGCGCTC
GTTGTTGAGGTAATGGAAGAGAACTATCATGACGCCCCCATCGTCGGTATAGCGCTGGTA
AACGAACATGGGCGATTTTTCATGCGGCCCGAAACAGCGTTGGCAGACAGTCAATTTCTT
GCCTGGCTTGCAGACGAGACGAAGAAAAAAAGCATGTTTGACGCGAAACGCGCGGTAGT
GGCACTCAAATGGAAGGGCATCGAGCTCAGGGGTGTAGCCTTCGATCTCCTGCTCGCTGC
GTACCTTCTTAATCCCGCGCAGGATGCAGGCGACATAGCCGCTGTCGCAAAGATGAAGC
AATATGAGGCGGTCCGATCCGATGAAGCCGTTTACGGCAAGGGCGTGAAACGGAGTCTC
CCTGATGAGCAAACACTTGCGGAACATCTTGTGCGAAAAGCCGCAGCGATATGGGCTCT
GGAACAGCCATTTATGGATGACTTGCGAAACAACGAGCAAGATCAGCTGTTGACGAAGT
TGGAACAACCGCTTGCGGCGATACTGGCGGAGATGGAATTCACGGGGGTGAACGTTGAT
ACGAAAAGGCTTGAGCAGATGGGATCAGAACTCGCTGAACAACTTAGAGCCATCGAACA
AAGAATATACGAACTTGCGGGGCAGGAATTCAATATAAATAGCCCAAAACAACTTGGGG
TCATACTCTTTGAGAAGCTTCAACTCCCCGTATTGAAAAAGACGAAGACGGGGTATAGTA
CAAGTGCGGATGTCCTGGAAAAGTTGGCGCCGCATCACGAAATTGTAGAAAATATACTG
CATTACAGGCAACTTGGGAAACTCCAATCAACGTACATAGAAGGACTCCTTAAAGTTGTC
CGACCTGATACAGGCAAGGTCCACACGATGTTTAATCAAGCACTTACGCAAACCGGTCG
CCTGAGCTCTGCGGAGCCAAATCTCCAGAATATACCGATTCGGCTGGAAGAAGGTCGCA
AAATTCGGCAGGCGTTCGTACCTAGCGAACCTGATTGGCTTATATTCGCGGCGGATTACT
CTCAGATAGAGCTTAGGGTATTGGCTCACATTGCCGATGACGACAACTTGATTGAAGCGT
TCCAGCGCGATTTGGACATACATACTAAGACAGCAATGGATATCTTCCACGTGTCTGAGG
AGGAGGTAACTGCTAACATGCGGCGGCAGGCAAAGGCCGTAAACTTTGGTATTGTTTAT
GGAATAAGCGACTACGGGCTCGCCCAGAACCTTAACATCACACGCAAAGAAGCCGCCGA
GTTTATTGAGAGATATTTCGCAAGTTTCCCCGGAGTAAAACAATACATGGAGAATATCGT
ACAAGAGGCTAAGCAGAAGGGCTATGTCACCACATTGCTCCACAGAAGACGGTATTTGC
CAGACATTACTAGTCGAAACTTTAACGTGAGGTCATTCGCAGAGCGGACGGCGATGAAT
ACACCCATTCAAGGAAGTGCAGCTGACATTATCAAAAAGGCCATGATTGACCTCGCAGC
TAGGTTGAAAGAAGAACAGCTCCAGGCCCGCCTGCTGCTCCAGGTGCATGATGAGCTCA
TACTCGAAGCCCCGAAGGAGGAAATAGAACGGCTGTGCGAGTTGGTCCCAGAAGTAATG
GAGCAAGCTGTCACGCTCCGAGTTCCCCTTAAGGTGGACTACCATTATGGTCCAACGTGG
TATGATGCTAAG
>SEQ ID No: 25 RAD51 ssDNA binding domain:
Gcgatgcagatgcagttggaagcgaatgcagatactagtgtcgaggaagagtcatttggcccgcaacccatctcgcgtttagagcaatgtggc
atcaatgcaaacgatgtgaaaaaattagaggaagctggattccacacggtcgaagcggtcgcatacgcaccgaaaaaagagctgatcaacatc
aaaggcatcagcgaggcgaaagccgataagattcttgcagaggcggcgaaattagttcccatgggatttacgacggcgactgagttccatcaa
cgtcgttccgagatcattcaaatcacgaccggaagcaaggagttggataaactgctt
>SEQ ID No: 26 RAD51D ssDNA binding domain:
GGCGTGCTCAGGGTCGGACTGTGCCCTGGCCTTACCGAGGAGATGATCCAGCTTCTCAGG
AGCCACAGGATCAAGACAGTGGTGGACCTGGTTTCTGCAGACCTGGAAGAGGTAGCTCA
GAAATGTGGCTTGTCTTACAAGGCCCTGGTTGCCCTGAGGCGGGTGCTGCTGGCTCAGTT
CTCGGCTTTCCCCGTGAATGGCGCTGATCTCTACGAGGAACTGAAGACCTCCACTGCCAT
CCTGTCC
>SEQ ID No: 27 RAD51AP1 ssDNA binding domain:
GGCAGTGATGGTGATAGTGCTAATGACACTGAACCAGACTTTGCACCTGGTGAAGATTCT
GAGGATGATTCTGATTTTTGTGAGAGTGAGGATAATGACGAAGACTTCTCTATGAGAAA
AAGTAAAGTTAAAGAAATTAAAAAGAAAGAAGTGAAGGTAAAATCCCCAGTAGAAAAG
AAAGAGAAGAAATCTAAATCCAAATGTAATGCTTTGGTGACTTCGGTGGACTCTGCTCCA
GCTGCCGTCAAATCAGAATCTCAGTCCTTGCCAAAAAAGGTTTCTCTGTCTTCAGATACC
ACTAGGAAACCATTAGAAATACGCAGTCCTTCAGCTGAAAGCAAGAAACCTAAATGGGT
CCCACCAGCGGCATCTGGAGGTAGCAGAAGTAGCAGCAGCCCACTGGTGGTAGTGTCTG
TGAAGTCTCCCAATCAGAGTCTCCGCCTTGGC
>SEQ ID No: 28 NEQ199 ssDNA Binding protein:
GACGAAGAGGAACTCATCCAGTTGATAATAGAAAAAACTGGTAAGTCCCGCGAAGAAAT
AGAGAAGATGGTTGAGGAGAAAATAAAGGCGTTCAACAATCTCATCTCACGAAGAGGA
GCTTTGCTCCTCGTGGCAAAGAAACTTGGAGTATTaTACAAGAACACGCCGAAGGAAAA
AAAAATTGGCGAGCTTGAATCCTGGGAGTATGTTAAGGTTAAAGGCAAGATACTGAAGA
GCTTTGGGCTTATTTCTTACAGCAAAGGCAAGTTCCAGCCCATTATTCTGGGAGACGAAA
CTGGCACAATTAAGGCGATTATATGGAACACCGACAAAGAATTGCCAGAGAACACAGTT
ATAGAAGCTATAGGTAAGACCAAGATCAACAAGAAAACTGGGAATCTTGAACTTCATAT
AGACTCCTATAAAATCCTCGAATCCGATCTTGAGATAAAACCTCAAAAGCAAGAATTTGT
TGGGATCTGTATTGTGAAGTACCCCAAGAAACAAACACAGAAAGGGACAATCGTTTCTA
AAGCGATATTGACCAGTCTCGATAGGGAACTTCCCGTGGTGTACTTCAATGACTTCGATT
GGGAAATTGGCCATATCTATAAGGTGTATGGAAAACTGAAAAAGAATATAAAAACGGGA
AAAATCGAGTTTTTCGCGGATAAGGTGGAAGAAGCCACGCTTAAGGATCTCAAAGCGTT
TAAGGGCGAAGCTGAC
>SEQ ID No: 29 PIF1:
AGTAGTCGTGGTTTCAGGTCTAATAACTTTATTCAAGCACAATTGAAGCATCCTTCCATA
CTTTCAAAAGAAGACCTAGATTTGCTCTCTGATTCGGATGATTGGGAAGAACCTGATTGC
ATACAGTTAGAAACTGAGAAGCAAGAAAAGAAAATTATCACTGACATACATAAAGAAG
ACCCGGTGGACAAAAAGCCTATGAGGGATAAAAATGTCATGAATTTTATCAATAAAGAC
AGTCCTTTATCCTGGAACGATATGTTTAAACCCAGTATAATACAACCACCGCAGTTAATT
TCTGAAAACTCATTTGACCAGAGCAGTCAAAAAAAATCGAGATCGACAGGATTCAAGAA
TCCATTAAGACCAGCGTTGAAAAAGGAAAGTTCTTTTGATGAACTTCAAAATAATTCTAT
ATCTCAAGAGAGAAGTTTGGAAATGATAAATGAAAACGAAAAGAAGAAAATGCAATTT
GGAGAAAAGATTGCTGTTTTGACGCAAAGACCTAGCTTCACTGAATTGCAGAATGACCA
AGATGACAGTAACTTGAATCCCCATAATGGTGTGAAAGTCAAGATACCGATTTGCTTAAG
CAAAGAACAAGAAAGTATCATCAAGTTGGCAGAAAATGGCCACAACATTTTTTATACAG
GGAGTGCCGGTACCGGTAAATCCATTCTTTTACGTGAAATGATAAAAGTTTTAAAAGGCA
TATATGGTAGGGAGAATGTTGCAGTCACTGCTTCCACGGGTTTAGCTGCTTGTAATATCG
GTGGTATAACCATACACTCGTTCGCTGGTATAGGATTAGGAAAAGGTGATGCGGATAAA
CTCTATAAAAAAGTTCGTAGGTCTCGAAAGCACCTAAGGCGCTGGGAAAATATTGGTGC
TTTGGTTGTCGATGAAATATCAATGTTAGACGCAGAACTGCTTGATAAACTCGATTTCAT
AGCTAGAAAAATACGGAAAAATCATCAACCCTTCGGTGGAATTCAACTCATCTTCTGTGG
CGATTTTTTCCAGTTACCGCCAGTATCAAAAGATCCTAATAGACCAACTAAGTTTGCTTTC
GAATCCAAGGCTTGGAAAGAAGGTGTAAAGATGACGATTATGCTACAAAAGGTTTTTAG
ACAGCGAGGCGATGTTAAGTTCATTGACATGTTGAATCGGATGAGACTAGGCAATATTG
ATGATGAAACAGAAAGAGAGTTCAAGAAGCTTTCTAGACCATTGCCAGACGATGAAATT
ATTCCCGCGGAACTTTATAGTACCAGAATGGAAGTAGAAAGGGCCAATAATTCAAGGCT
AAGTAAATTGCCAGGCCAGGTGCATATTTTTAATGCAATCGATGGCGGTGCTTTGGAAGA
CGAAGAGTTAAAGGAAAGGCTGTTACAAAATTTTTTAGCTCCAAAGGAATTACATTTGA
AAGTTGGCGCTCAGGTTATGATGGTAAAAAATCTAGACGCAACATTAGTTAATGGATCCC
TTGGTAAAGTCATCGAATTCATGGATCCAGAAACATATTTTTGCTATGAGGCGCTAACAA
ACGATCCATCTATGCCTCCAGAAAAACTCGAGACTTGGGCAGAAAACCCTTCAAAACTA
AAAGCTGCAATGGAGAGGGAGCAAAGTGATGGGGAAGAAAGTGCGGTAGCTAGTCGCA
AATCTTCAGTGAAGGAGGGATTTGCTAAGAGTGATATAGGTGAGCCGGTCTCTCCCCTAG
ATTCCTCAGTTTTTGACTTCATGAAGAGAGTCAAGACAGATGACGAAGTTGTGCTGGAAA
ATATAAAACGCAAGGAACAACTGATGCAGACCATACATCAAAACTCTGCAGGAAAACGA
AGGTTACCTCTCGTGAGATTCAAAGCTTCTGATATGAGTACGAGGATGGTGCTTGTCGAG
CCGGAGGATTGGGCGATAGAAGACGAAAATGAAAAGCCACTGGTATCAAGGGTTCAATT
ACCGCTAATGCTTGCCTGGTCACTATCCATTCACAAATCTCAGGGTCAGACACTTCCAAA
AGTTAAAGTGGATTTACGTAGAGTATTCGAAAAGGGTCAGGCGTAtGTTGCCCTTTCTAG
AGCTGTTTCAAGAGAAGGACTACAGGTGTTAAATTTTGACAGAACTAGGATCAAAGCAC
ATCAAAAGGTAATTGATTTTTATCTTACTTTATCTTCAGCCGAAAGTGCCTATAAGCAACT
TGAGGCAGATGAGCAAGTGAAAAAAAGGAAGTTAGACTACGCACCAGGCCCTAAATAT
AAGGCTAAATCCAAGTCAAAGTCAAATTCTCCAGCACCCATATCAGCGACCACACAATC
TAATAATGGTATCGCAGCGATGTTGCAAAGACACAGTAGGAAGAGATTTCAGTTGAAAA
AAGAGTCTAATAGTAATCAAGTTCATTCATTGGTTTCCGACGAACCTCGTGGTCAGGATA
CCGAAGACCACATCTTAGAA
>SEQ ID No: 30 RTX:
attcttgacacggattacatcacggaagacggcaagccggttatccgtattttcaagaaagaaaacggcgaattcaagattgaatacgatcgg
acatttgaaccgtacctgtacgctctcctcaaggatgatagcgcaatcgaagaagtgaaaaaaatcaccgcagagcggcatggcacagtggta
acagttaagcgggtcgagaaagtgcagaagaagttcttaggccggccagtcgaagtatggaaattatacttcacacatccacaggacgttccg
gcgatcatggataagattcgggagcatccggcggtaatcgatatctatgaatacgatattccgttcgctattcgctaccttattgacaaaggt
ttagttccaatggagggtgatgaggaacttaaactgttagcattcgatatcgaaacactttatcacgaaggtgaagagtttgccgaaggtccg
attttaatgatctcAtacgccgatgaagaaggcgcacgcgtaattacgtggaaaaatgtggacctcccAtacgtagacgtagtgagcactgag
cgcgagatgattaaacgtttccttcgggtagtaaaagaaaaagacccagacgtgctgattacgtataacggcgacaactttgattttgcctat
ctcaagaagcgttgcgaaaagttaggcattaatttcgccctgggtcgggacggttcagagccgaaaattcagcggatgggcgaccgctttgct
gtggaggtaaaaggtcgcatccatttcgatttatatccggttatccggcgcaccatcaacttgccgacttacacacttgaagcagtttacgaa
gcggtgttcggccaaccaaaagaaaaggtttatgccgaggagattaccaccgcatgggaaactggcgaaaacttggagcgggtggctcggtat
tccatggaagatgccaaggtgacctacgaactgggcaaagagtttttaccgatggaagcacaattaagccgccttattggtcagtccctctg
ggatgtgtcgcgttcttcaacgggcaatttagtcgaatggtttcttcttcggaaagcAtacgagcgtaacgagcttgctccaaataagccag
acgaaaaagaattggctcggcgccatcagtcacatgagggcggctacattaaggagccagaacggggcttgtgggagaacatcgtctacctt
gattttcggtctctttatccgtctattatcatcacacataacgtctcgccagataccctgaaccgtgaaggctgtaaagaatatgatgtggca
ccacaggtcggccatcgtttttgtaaagacttcccgggcttcattccatctcttctgggtgatttgttagaagagcgtcaaaagatcaagaaa
cgtatgaaagcgacaattgacccaattgaacgcaaattacttgattaccgtcagcgtgcaatcaagatcctcgcgaactctctgtacggtta
ttacggctacgcacgcgcccggtggtattgcaaagaatgtgcagaatcagtcattgcttggggtcgggagtacctgaccatgacgattaagga
aattgaggagaaatacggtttcaaggtcatctatagtgacacggatggtttctttgcaacgattccaggtgcggacgcagaaactgtaaagaa
aaaggcaatggagttcttgaagtatattaatgcgaagttgccaggcgccctggaattagagtacgaaggtttttataagcgtggcctgttcg
tgacaaagaagaaatacgcggtaattgacgaggaaggcaagatcacaactcgtggcttggaaattgttcgtcgcgattggagcgagatcgca
aaggagacccaagctcgtgtgttggaggccctcctgaaggatggtgacgtcgaaaaagcAgtacgcatcgttaaggaggttacagagaagct
tagcaagtatgaggtcccaccagagaaacttgttattcataaacaaatcactcgcgaccttaaagactataaggccactggtccacacgtcg
ccgtagcaaagcggcttgcggctcggggcgtcaagattcggccaggcacggttattagttacatcgtcctcaaaggctcaggccggattgtt
gatcgcgcgattccatttgatgaatttgatccgacgaagcataaatatgatgcggaatattacattgaaaaacaggttctgccggcggtgga
gcgcatcttacgtgcgttcggctatcgcaaggaggatttgcggtaccagaaaactcgtcaagtcggtttgagtgcctggctgaagccgaaag
gtacctga
>SEQ ID No: 31 M160 reverse transcriptase:
AACACACCAAAACCCATTCTCAAACCGCAATCTAAGGCCTTGGTAGAGCCCGTACTTTGT
GATTCTATCGACGAGATCCCGGCCAAGTACAACGAGCCCGTGTATTTTGACTTGGaAACG
GATGAAGATCGACCAGTACTCGCATCCATATATCAACCTCATTTTGAAAGGAAAGTCTAT
TGTCTCAACTTGCTGAGGGAAAAGTTGGCCCGCTTTAAGGAGTGGCTTCTCAAGTTTTCC
GAGATCCGAGGGTGGGGACTTGACTTCGACCTCCGAGTGTTGGGCTACACATACGAACA
GCTGAGGAATAAGAAGATTGTAGACGTCCAACTCGCGATAAAGGTACAGCACTATGAGC
GATTCAAGCAAGGAGGGACGAAGGGAGAAGGCTTTAGATTGGACGACGTTGCCCGAGAT
CTGTTGGGTATCGAGTATCCAATGAACAAAACGAAAATAAGAACGACCTTTAAGTATAA
CATGTACTCTAGCTTCTCTTACGAGCAATTGCTGTACGCAAGCCTCGACGCATACATTCCT
CACCTGCTGTATGAGAGGCTTAGCAGTGACACGCTCAATTCTTTGGTATACCAAATAGAT
CAAGAGGTGCAGAAAGTTGTCATAGAAACATCTCAGCATGGCATGCCCGTAAAACTGAA
AGCACTGGAGGAAGAAATACATAGACTCACACAGCTTAGGTCAGAAATGCAAAAACAG
ATTCCCTTCAACTACAATTCTCCTAAGCAGACAGCGAAGTTTTTCGGCGTTAACTCTTCTT
CAAAGGACGTCCTCATGGATCTTGCCCTCAGGGGCAACGAAGTTGCGAAAAAAGTGCTG
GAGGCAAGACAAATCGAGAAGTCCCTGGCATTCGCGAAGGACCTCTACGATATAGCCAA
GAAAAATGGCGGCCGAATTTATGGAAATTTCTTCACGACGACAGCCCCCAGCGGAAGGA
TGAGCTGCTCAGATATCAATTTGCAGCAGATCCCGCGACGGCTTAGGCCGTTCATAGGTT
TTGAAACGGAGGATAAGAAGCTTATCACCGCTGACTTCCCACAGATCGAACTTCGGCTG
GCTGGGGTTATGTGGAACGAACCTGAGTTCCTGAAAGCCTTTCGGGACGGAATAGATCTC
CATAAATTGACGGCCAGCATTCTCTTCGATAAAAAAATAAATGAGGTGAGCAAAGAAGA
GCGCCAAATTGGTAAATCAGCGAATTTTGGCTTGATTTACGGAATTTCTCCGAAAGGGTT
CGCGGAGTATTGCATCTCCAATGGAATCAATATAACAGAGGAGATGGCAATCGAAATCG
TCAAGAAATGGAAGAAGTTCTATCGCAAGATAGCCGAACAGCACCAACTCGCCTACGAA
CGGTTCAAATACGCTGAGTTCGTTGATAATGAAACCTGGTTGAACAGGCCCTATCGCGCT
TGGAAACCCCAGGACCTCCTCAACTATCAAATCCAAGGCAGTGGAGCTGAACTCTTCAA
GAAAGCAATCGTGTTGTTGAAAGAAGCAAAGCCAGATCTCAAAATTGTGAACCTCGTGC
ATGATGAAATAGTGGTCGAGACCTCCACCGAGGAAGCAGAAGATATTGCACTCCTTGTT
AAACAAAAGATGGAAGAGGCTTGGGACTACTGCCTGGAGAAGGCCAAGGAATTTGGTA
ATAACGTCGCTGATATTAAGCTTGAGGTTGAGAAACCAAACATATCCAGCGTCTGGGAA
AAAGAA
>SEQ ID No: 32 MMULV reverse transcriptase
accctaaatatagaagatgagtatcggctacatgagacctcaaaagagccagatgtttctctagggtccacatggctgtctgattttcctca
ggcctgggcggaaaccgggggcatgggactggcagttcgccaagctcctctgatcatacctctgaaagcaacctctacccccgtgtccataa
aacaataccccatgtcacaagaagccagactggggatcaagccccacatacagagactgttggaccagggaatactggtaccctgccagtcc
ccctggaacacgcccctgctacccgttaagaaaccagggactaatgattataggcctgtccaggatctgagagaagtcaacaagcgggtgga
agacatccaccccaccgtgcccaacccttacaacctcttgagcgggctcccaccgtcccaccagtggtacactgtgcttgatttaaaggatg
cctttttctgcctgagactccaccccaccagtcagcctctcttcgcctttgagtggagagatccagagatgggaatctcaggacaattgacc
tggaccagactcccacagggtttcaaaaacagtcccaccctgtttaatgaggcactgcacagagacctagcagacttccggatccagcaccc
agacttgatcctgctacagtacgtggatgacttactgctggccgccacttctgagctagactgccaacaaggtactcgggccctgttacaa
acActagggaacctcgggtatcgggcctcggccaagaaagcccaaatttgccagaaacaggtcaagtatctggggtatcttctaaaagaggg
tcagagatggctgactgaggccagaaaagagactgtgatggggcagcctactccgaagacccctcgacaactaagggagttTctagggaagg
caggcttctgtcgcctcttcatccctgggtttgcagaaatggcagcccccctgtaccctctcaccaaaccggggactctgtttaattggggc
ccagaccaacaaaaggcctatcaagaaatcaagcaagctcttctaactgccccagccctggggttgccagatttgactaagccctttgaact
ctttgtcgacgagaagcagggctacgccaaaggtgtcctaacgcaaaaactgggaccttggcgtcggccggtggcctacctgtccaaaaagc
tagacccagtagcagctgggtggcccccttgcctacggatggtagcagccattgccgtactgacaaaggatgcaggcaagctaaccatggga
cagccactagtcattctggccccccatgcagtagaggcactagtcaaacaaccccccgaccgctggctttccaacgcccggatgactcacta
tcaggccttgcttttggacacggaccgggtccagttcggaccggtggtagccctgaacccggctacgctgctcccactgcctgaggaagggc
tgcaacacaactgccttgatatcctggccgaagcccacggaacccgacccgacctaacggaccagccgctcccagacgccgaccacacctgg
tacacggatggaagcagtctcttacaagagggacagcgtaaggcgggagctgcggtgaccaccgagaccgaggtaatctgggctaaagccct
gccagccgggacatccgctcagcgggctgaactgatagcactcacccaggccctaaagatggcagaaggtaagaagctaaatgtttatactg
atagccgttatgcttttgctactgcccatatccatggagaaatatacagaaggcgtgggtggctcacatcagaaggcaaagagatcaaaaat
aaagacgagatcttggccctactaaaagccctctttctgcccaaaagacttagcataatccattgtccaggacatcaaaagggacacagcgc
cgaggctagaggcaaccggatggctgaccaagcggcccgaaaggcagccatcacagagactccagacacctctaccctcctcatagaaaatt
catcaccctctggcggctcaaaaagaaccgccgacggcagcgaattcgagcccaagaagaagaggaaagtc
>SEQ ID No: 33 MAGMA DNA polymerase
CGCGGAATGCTCCCACTCTTCGAACCTAAGGGCAGAGTTCTTCTTGTTGACGGACACCAC
TTGGCATATAGAACATTCCATGCACTCAAAGGGCTCACGACCTCACGGGGAGAACCTGT
GCAAGCTGTGTACGGTTTTGCCAAGAGTTTGTTGAAGGCCCTCAAGGAGGATGGTGATGC
TGTAATAGTTGTATTTGATGCCAAGGCTCCTTCTTTCCGACATGAGGCTTATGGCGGCTAT
AAGGCTGGGCGGGCGCCTACACCAGAAGATTTTCCTCGACAACTGGCGTTGATCAAAGA
GTTGGTTGATTTGCTCGGACTCGCCCGACTTGAGGTTCCGGGATACGAAGCCGACGACGT
GTTGGCATCTTTGGCAAAGAAGGCGGAAAAAGAAGGATACGAGGTACGGATTCTTACAG
CTGACAAGGATCTGTACCAGTTGTTGTCAGATCGCATACACGTTTTGCATCCCGAGGGTT
ACCTTATTACACCCGCCTGGCTCTGGGAGAAATACGGCCTTCGGCCCGACCAATGGGCTG
ATTATCGAGCCCTGACGGGTGACGAATCAGATAACCTGCCCGGCGTTAAAGGGATTGGT
GAGAAAACGGCCCGAAAGTTGCTTGAAGAATGGGGCTCTTTGGAGGCACTTCTCAAGAA
CCTGGACCGCCTGAAACCTGCCATCCGCGAAAAAATACTCGCACACATGGATGATCTCA
AACTCAGCTGGGACTTGGCGAAAGTCCGAACAGATCTGCCTCTCGAAGTGGACTTTGCA
AAGAGGCGGGAGCCAGACAGGGAACGACTCAGGGCCTTCCTGGAACGACTGGAATTTGG
ATCATTGTTGCACGAGTTCGGACTCCTGGAATCTGGTGGTGGAGGTTCTGGTGGTGGTGG
CAGCAACACACCAAAACCCATTCTCAAACCGCAATCTAAGGCCTTGGTAGAGCCCGTAC
TTTGTGATTCTATCGACGAGATCCCGGCCAAGTACAACGAGCCCGTGTATTTTGACTTGGa
AACGGATGAAGATCGACCAGTACTCGCATCCATATATCAACCTCATTTTGAAAGGAAAG
TCTATTGTCTCAACTTGCTGAGGGAAAAGTTGGCCCGCTTTAAGGAGTGGCTTCTCAAGT
TTTCCGAGATCCGAGGGTGGGGACTTGACTTCGACCTCCGAGTGTTGGGCTACACATACG
AACAGCTGAGGAATAAGAAGATTGTAGACGTCCAACTCGCGATAAAGGTACAGCACTAT
GAGCGATTCAAGCAAGGAGGGACGAAGGGAGAAGGCTTTAGATTGGACGACGTTGCCC
GAGATCTGTTGGGTATCGAGTATCCAATGAACAAAACGAAAATAAGAACGACCTTTAAG
TATAACATGTACTCTAGCTTCTCTTACGAGCAATTGCTGTACGCAAGCCTCGACGCATAC
ATTCCTCACCTGCTGTATGAGAGGCTTAGCAGTGACACGCTCAATTCTTTGGTATACCAA
ATAGATCAAGAGGTGCAGAAAGTTGTCATAGAAACATCTCAGCATGGCATGCCCGTAAA
ACTGAAAGCACTGGAGGAAGAAATACATAGACTCACACAGCTTAGGTCAGAAATGCAAA
AACAGATTCCCTTCAACTACAATTCTCCTAAGCAGACAGCGAAGTTTTTCGGCGTTAACT
CTTCTTCAAAGGACGTCCTCATGGATCTTGCCCTCAGGGGCAACGAAGTTGCGAAAAAA
GTGCTGGAGGCAAGACAAATCGAGAAGTCCCTGGCATTCGCGAAGGACCTCTACGATAT
AGCCAAGAAAAATGGCGGCCGAATTTATGGAAATTTCTTCACGACGACAGCCCCCAGCG
GAAGGATGAGCTGCTCAGATATCAATTTGCAGCAGATCCCGCGACGGCTTAGGCCGTTC
ATAGGTTTTGAAACGGAGGATAAGAAGCTTATCACCGCTGACTTCCCACAGATCGAACTT
CGGCTGGCTGGGGTTATGTGGAACGAACCTGAGTTCCTGAAAGCCTTTCGGGACGGAAT
AGATCTCCATAAATTGACGGCCAGCATTCTCTTCGATAAAAAAATAAATGAGGTGAGCA
AAGAAGAGCGCCAAATTGGTAAATCAGCGAATTTTGGCTTGATTTACGGAATTTCTCCGA
AAGGGTTCGCGGAGTATTGCATCTCCAATGGAATCAATATAACAGAGGAGATGGCAATC
GAAATCGTCAAGAAATGGAAGAAGTTCTATCGCAAGATAGCCGAACAGCACCAACTCGC
CTACGAACGGTTCAAATACGCTGAGTTCGTTGATAATGAAACCTGGTTGAACAGGCCCTA
TCGCGCTTGGAAACCCCAGGACCTCCTCAACTATCAAATCCAAGGCAGTGGAGCTGAAC
TCTTCAAGAAAGCAATCGTGTTGTTGAAAGAAGCAAAGCCAGATCTCAAAATTGTGAAC
CTCGTGCATGATGAAATAGTGGTCGAGACCTCCACCGAGGAAGCAGAAGATATTGCACT
CCTTGTTAAACAAAAGATGGAAGAGGCTTGGGACTACTGCCTGGAGAAGGCCAAGGAAT
TTGGTAATAACGTCGCTGATATTAAGCTTGAGGTTGAGAAACCAAACATATCCAGCGTCT
GGGAAAAAGAA
>SEQ ID No: 34 Foamy virus reverse transcriptase:
caagtcgggcatagaaaaattaggccacataatatagcaactggtgattatcctcctcgccctcaaaaacaatatcctattaatcctaaggc
aaagcctagtatacaaattgtaatagatgacttattgaaacaaggggtgttaacgcctcaaaatagtacaatgaatacaccagtgtatcctg
ttcctaaaccagatggaaggtggagaatggtattagattatagagaagtaaataaaactattccattaacagctgcccaaaaccaacactct
gctggtattttagctactattgttagacaaaaatataaaactaccttagatttagctaatggattttgggctcatcctattacaccagaatc
ttattggttaacagcatttacctggcaaggtaaacagtattgttggacacgtcttcctcaaggatttttaaatagtccagcattgtttacag
ctgatgtagtagatttactaaaagaaatccctaaCgtacaagtgtatgttgatgatatatatttaagccatgatgatcctaaagagcatgtt
caacaattagaaaaagtgtttcaaattttactacaggcaggatatgtagtatctttgaaaaaatcagaaattggtcaaaaaactgtagaat
ttttaggatttaatattactaaagaaggtcgtggcctaacagacacttttaaaacaaaactgttaaatattactcctccaaaagacttaaa
gcaattacaaagcatattaggattgttaaattttgctagaaattttatacctaattttgctgaactggtacaaccattatacaatttaatag
cctcagcaaaaggcaaatatattgagtggtctgaagaaaatactaaacaattaaatatggtaatagaagcattaaacactgcctctaattt
agaagaaaggttaccagaacagagactggtaattaaagtcaatacttctccatcagcaggatatgtaagatattataatgagactggtaaa
aagcctattatgtacctaaattatgtgttttccaaagcagaattaaaattttctatgttagaaaaactattaactacaatgcacaaagcct
taattaaggctatggatttggccatgggacaagaaatattagtttatagtcccattgtatctatgactaaaatacaaaaaactccactacc
agaaagaaaagctttacccattagatggataacatggatgacttatttagaagatccaagaatccaatttcattatgataaaaccttacca
gaacttaagcatattccagatgtatatacatctagtcagtctcctgttaaacatccttctcaatatgaaggagtgttttatactgatggct
cggccatcaaaagtcctgatcctacaaaaagcaataatgctggcatgggaatagtacatgccacatacaaacctgaatatcaagttttgaa
tcaatggtcaataccactaggtaatcatactgctcagatggctgaaatagctgcagttgaatttgcctgtaaaaaagctttaaaaatacc
tggtcctgtattagttataactgatagtttctatgtagcagaaagtgctaataaagaattaccatactggaaatctaatgggtttgttaat
aataagaaaaagcctcttaaacatatctccaaatggaagtctattgctgagtgtttatctatgaaaccagacattactattcaacatgaaa
aagggcatcagcctacaaataccagtattcatactgaaggcaatgccctagcagataagcttgccacccaaggaagttat
>SEQ ID No: 35 Bordetella bacteriophage reverse transcriptase
GGAAAAAGGCACAGGAACCTTATAGATCAGATTACGACGTGGGAAAATCTCTTGGACGC
GTACCGAAAAACTAGCCACGGTAAAAGACGAACATGGGGTTACCTGGAGTTCAAAGAGT
ACGACTTGGCAAATTTGTTGGCGCTCCAAGCGGAACTGAAGGCTGGAAACTACGAAAGA
GGCCCTTACCGCGAATTTCTGGTATATGAACCGAAACCACGGCTTATATCTGCTCTTGAA
TTCAAGGATAGACTCGTGCAGCATGCACTTTGTAATATAGTTGCCCCGATATTTGAAGCG
GGGCTTCTGCCATATACATACGCATGTCGGCCGGACAAGGGGACTCATGCGGGCGTTTGT
CATGTCCAGGCAGAGCTTCGACGAACACGAGCGACTCATTTTCTCAAATCCGATTTCAGT
AAATTCTTCCCCAGTATTGATCGAGCGGCTCTTTATGCCATGATCGACAAAAAGATTCAC
TGCGCCGCCACTCGGAGACTCTTGAGGGTGGTCCTGCCGGATGAAGGAGTAGGCATACC
GATTGGTAGCCTGACGAGTCAACTTTTTGCCAACGTATACGGCGGGGCAGTGGATCGCCT
TCTTCACGATGAACTTAAACAACGCCATTGGGCTAGGTATATGGATGACATCGTGGTTTT
GGGGGATGATCCCGAAGAATTGCGAGCGGTGTTCTACCGGCTTCGAGACTTCGCCAGCG
AGAGACTTGGCCTTAAAATAAGTCATTGGCAGGTTGCCCCCGTGAGCAGGGGCATAAAT
TTCCTGGGCTATCGGATTTGGCCGACGCATAAGCTCCTTCGAAAGTCTAGTGTCAAGAGG
GCCAAAAGAAAGGTAGCAAACTTTATTAAACACGGCGAGGACGAAAGTCTTCAGCGCTT
CTTGGCGAGCTGGAGCGGGCATGCCCAATGGGCTGACACGCACAATTTGTTCACTTGGAT
GGAGGAGCAGTACGGAATCGCGTGTCATtag
>SEQ ID No: 36 Treponema DGR reverse transcriptase
AAACGCAAGGGCAACTTGTATCACAAAATTACAGAATGGAACAACCTGATAGCCGCATT
TTACAACGCTAGTAGAGGCAAGAGGCTTAAGCCGGATGTCCTGCTGTACGAAAAGAACC
TTTACACAAATTTGAAGACCCTGCAAAATTATCTGATAAACCAGACCGTTCTCCTCGGTA
GCTACCGGTTTTTCAAAATTTACGATCCGAAGGAACGCATCATATGTGCGGCCCCGTTCA
ATGAACGAGTACTTCACCACGCGATAATAAATATAACAGAGAGCGTCTTTGAAAAGTTC
CAAATTTACGATTCCTACGCTTGTAGAAAAAACAAGGGGACGCAAGCCGCATTGTTGAG
GGCTCTCTACTTTTCCCGGCGGTTCAAATACTTCCTGAAATTGGATATGAAAAAGTACTTT
GATTCTATACCTCATTCCAAGCTCTCCCTGCTTCTGACCTGCAAATTCAAGGATAAGGCG
TTGCTGCATTTGTTTAACAAACTTATCGCATCTTACAGCGTAACTGAAGGGTGGGGCGTG
CCTATAGGCAATTTGACGAGTCAGTACTTCGCCAATTTTTATCTGTCTTTTTTCGATCACT
ATGCTAAGGAAAAAATGAATGTCCGGGGGTATATCCGGTACATGGATGATGTGCTGTTG
TTCTCCGATAACCTCAAAGATATTAAACTGATCCAAAAGAAAGCTAAAAATTTTCTCAGC
TGCGAACTGGATCTCACCTTGAAGGAGGAGATAATTGGTATGGTGAAGAATGGCATCCC
GTTTCTCGGATTCCTCGTGAAACCACAAGGGATCTACTTGAGCCAAAAAAAGAAGAAAA
GGCTGAAGAAGAAAATTAAAGATTACGTTCACAAGTTTAAGATTGCTTATTGGACGGAG
GAGGAGTTTGCTTTGCACATTACGCCAGTTTTCGCCCACATTGCGATATCCCGATGTCGC
GCATACTGTAACAAATACCTCTTGACAtag
>SEQ ID No: 37 Bacteroides DGR reverse transcriptase
TGGAGGGAAGACAATATTATCGAAGAAATAGTCGAAGATAGCAACATCGAAGATGCGAT
AAAGACCGTACTGAGGAAGCGCAGGCGAAAACGGTCATTTGCGGGTCGCAGGATTCTGG
CGGATGTCCCAAAAGCGGTGGAGCGGATTAGGAAAAGGATACGAAGTGGGAGGTTTAA
GCTCGGTGGCTACAGAGAGATGACGGTAGACGATGGGCCCAAGGTGCGCATAGTTCAGG
CCGTGAGCCTCGAAGACCGCATCGTTCTTAATGCCGTCATGAATGTAGTAGATAGGCACT
TGAAGGTCAGATTCATACGCACGACCAGTGCCTCCATCAAGAACCGAGGCACTCACGAT
CTCCTCCAATATATCGTGAAGGATATTAAGGACGATCCTGAGGGGACGCTTTTCGGCTAT
CAATTTGACATAACGAAATTTTACGAGTCAGTTGACCAGGATGTGCTGCTCGACGCCGTA
AAACGCATGTTTAAAGACAAAATCTTGATAGGTATCCTCGAAGAATGCATCAGAATGAT
GCCTAAGGGGGTATCAATCGGATTGAGATCCTCCCAGGGCCTCTGCAACCTTCTCCTCTC
TATATATTTGGATCATCGGCTTAAAGATCAAGAGGCTGTCGCACATTATTACAGGTATTG
CGATGACGGTCTCGTCCTCAGCGGCTCTAAAAAATATTTGTGGAAAGTCCGGGATATCAT
CCACGAACAAACTAGGAAAGCCCGGTTGGAAATAAAATCTAATGATACTGTGTTCCCTA
TCACAGAAGGAATCGATTTCCTTGGTTACGTCACCAGGCCCGATCACGTGAGGCTCAGAA
AGCGGAATAAGCAAAAATTCGCCCGCAAAATGCACAAGATTAAATCAAAGAAGCGCCG
CCAAGAGCTGACAGCTTCTTTTTACGGTTTGACTAAGCATGCGGACTGTAAAAACTTGTT
CTATAAGCTGACAGGCAAGAAAATGAAGAAGCTTAAAGATTTGGGATACAAGTACAAGC
CCAAGGATGGAAGAAAGCGGTTTACAGGGACCCGAATCAAATCTCCCGAACTGATGAAC
AAGGATGTAATCGTTTTGGATTATGAAAAAGATGTCCCTACCAAGAATGGTAATCGAAC
AGTTATCAAACTGGAGCTCGATGGCAAGGAACGGAAGTATTTCACGTCTCTCGAAGAAA
CTCTCTTTATATGTGAATCTGCTGCGAAGGATGGCGAACTGCCATTTGAGGCCCATTGTG
AGGGGGAAGTATCCGAGAAAGGTCTCATTATCATTCACTTCACAtag
>SEQ ID No: 38 Eggerthella lenta DGR reverse transcriptase gene:
AACTCAGATGAACGCAGGGCCGCAAGACGCGCGAGAAGAGAAGCTGAGCGGGCACGAC
GCAAAGCAGAGCGCAACGCAGGTTGTGACCTCGAAGCAGTGGCCGATCTTAATGCTCTC
TACAAAGCGGCGAAACAGGCGGCCCGAGGAGTGGCATGGAAGGCATCAGTTCAAAGAT
ATCAGGCTGATGTTTTGCGAAACGTAATGAAGGCTCGGAGAGACTTGCTTGAGGGGAGG
GATGTCTGTCGAGGATTCATAAGGTTCGACCTCTGGGAGCGCGGGAAGCTTAGGCACAT
CAGTGCGGTACGATTTAGTGAACGGGTCATACAAAAAAGTCTCACACAGAATGCACTGG
TTCCAGCTATAGCACCGACACTCACGTATGACAATTCAGCAAACTTGAAAGGGAAAGGA
ACTGACTTTGCCATTGCACGGATGAAAAAGCAGTTGGCTAGATTTTATAGGAAACACGG
CGCCGATGGGTATATCCTGCTGGTGGATTTTTCTGATTACTTCGCAAGAATCTCTCATGGC
CCTGCTAAGGCAATTGTTGCTGGGGCCCTTGAGGATAGGCGGCTCGTAGCGTTGGAACAC
CGGTTCATTGACGCACAGGGAGACATTGGGCTCGGTCTCGGCAGTGAACCCAACCAGAT
TCTTGCTGTAGCATTTCCATCTTATATAGATCACTTCGCAGCTGAAATGTGCGGACTGGA
GGCCACCGGCCGGTATATGGATGACTCATATTATATACACGAGTCTAAAGCATATCTCGA
AGTTGTATTGATGCTGATAGAGCAGAAGTGCGATCAATGTGGCATTTCAATCAATAGAA
AGAAGACAAGAATCGTAAAACTGTCCCGAGGGTTCACATTCCTGAAAAAGAAAATTTCC
TTTGGTGAGAATGGGAGAATCGTAGTCCGCCCATCACGAGAGAGTATAACACGCGAGCG
ACGGAAACTGAAGAAACAAAGAAAACTTGTCGACCTGGGTATGATGACTCCAGAACAGG
TGGAACGCAGTTATCAGAGTTGGAGAGGCGGCATGAAAAAGTTGGATGCGCATAGAACG
GTACTGTCCATGGACGCATTGTATAAAGATCTCTTCTCAAACCCTGAAAATGCGTCAAGG
GGTGGAGTGTCATTGAAATAA
>SEQ ID No: 39 CDT degron
AGCACTGACGTTGAGCCTAGCCCTGCACGGCCGGCATTGCGGGCACCCGCCTCAGCTACT
AGCGGGAGCAGGAAGAGAGCCAGGCCCCCTGCAGCACCTGGCAGGGACCAGGCCAGGC
CACCCGCTCGCAGACGACTTCGCCTGTCCGTCGATGAGGTCTCATCCCCTTCCACCCCCG
AAGCACCTGACATACCCGCCTGTCCTAGTCCCGGTCAGAAGATTAAGAAATCCACCCCCG
CCGCCGGCCAACCACCCCACCTGACCAGCGCCCAGGATCAGGACACCATT
>SEQ ID No: 40 CDT degron tandem copy:
AGCACTGACGTTGAGCCTAGCCCTGCACGGCCGGCATTGCGGGCACCCGCCTCAGCTACT
AGCGGGAGCAGGAAGAGAGCCAGGCCCCCTGCAGCACCTGGCAGGGACCAGGCCAGGC
CACCCGCTCGCAGACGACTTCGCCTGTCCGTCGATGAGGTCTCATCCCCTTCCACCCCCG
AAGCACCTGACATACCCGCCTGTCCTAGTCCCGGTCAGAAGATTAAGAAATCCACCCCCG
CCGCCGGCCAACCACCCCACCTGACCAGCGCCCAGGATCAGGACACCATTGGAAGCGGC
TCTGGCAGTACCGACGTGGAACCATCTCCAGCTCGACCCGCCCTCAGGGCCCCAGCATCT
GCGACAAGTGGCAGTCGCAAGAGAGCACGGCCTCCTGCCGCACCCGGTCGGGACCAGGC
ACGCCCCCCCGCAAGACGCCGACTTAGACTGTCAGTTGATGAAGTGTCCAGCCCCTCTAC
ACCTGAGGCACCTGATATTCCTGCTTGCCCAAGTCCTGGACAGAAAATCAAGAAGAGCA
CGCCCGCCGCAGGTCAGCCTCCACACCTCACGTCTGCGCAGGACCAAGACACCATT
>SEQ ID No: 41 scFV S9.6 protein:
GACATAGTTATGACTCAAACCCCGCTTTCCCTCCCAGTCTCACTGGGGGATCAAGCGTCC
ATCTCATGCCGCTCTTCACAGAGTATTGTGCATTCTAACGGTAACACATACCTGGAATGG
TATTTGCAAAAGCCAGGTCAAAGCCCAAAGCTTCTCATCTATAAGGTTTCAAATAGGTTT
TCTGGCGTCCCAGATCGATTCTCCGGGAGTGGGTCTGGTACTGATTTTACTCTTAAGATAT
CAAGAGTCGAGGCCGAGGACTTGGGGGTCTATTACTGTTTCCAAGGGAGCCACGTTCCAT
ATACTTTTGGGGGTGGGACAAAACTGGAAATAAAACGAGGGGGCGGAGGGTCCGGAGG
AGGGGGGAGTGGCGGAGGAGGGTCAGGTGGCGGAGGATCCCAGGTGCAGTTGCAACAG
TCAGGTCCAGAATTGGTTAAACCTGGCGCGTCTGTAAAAATGTCCTGTAAAGCGTCCGGA
TACACGTTTACGAGTTACGTTATGCACTGGGTGAAACAGAAACCGGGGCAGGGCCTGGA
ATGGATCGGGTTTATCAACTTaTACAACGATGGAACAAAGTACAATGAAAAGTTTAAAGG
CAAAGCCACGTTGACTTCAGATAAAAGCTCATCAACTGCATATATGGAGCTGTCATCTCT
TACTTCCAAGGATAGCGCGGTTTATTACTGTGCTCGGGATTATTATGGAAGCAGATGGTT
TGACTATTGGGGACAAGGGACGACATTGACTGTATCTAGC
>SEQ ID No: 42 Protein G B1 domain (GB1):
GGTGGAGGTCGGACCGAAGAGTACAAGCTTATCCTGAACGGTAAAACCCTGAAAGGTGA
AACCACCACCGAAGCTGTTGACGCTGCTACCGCGGAAAAAGTTTTCAAACAGTACGCTA
ACGACAACGGTGTTGACGGTGAATGGACCTACGACGACGCTACCAAAACCTTCACGGTA
ACCGAAGGTGGTGGTAGCGGTGGTGGTACTAGTCCCAAGAAGAAGCGCAAGGTG
>SEQ ID No: 43 Maltose Binding Protein (MBP):
TCTAACCAAATATACTCAGCGAGATATTCGGGGGTTGATGTTTATGAATTCATTCATTCT
ACAGGATCTATCATGAAAAGGAAAAAGGATGATTGGGTCAATGCTACACATATTTTAAA
GGCCGCCAATTTTGCCAAGGCTAAAAGAACAAGGATTCTAGAGAAGGAAGTACTTAAGG
AAACTCATGAAAAAGTTCAGGGTGGATTTGGTAAATATCAGGGTACATGGGTCCCACTG
AACATAGCGAAACAACTGGCAGAAAAATTTAGTGTCTACGATCAGCTGAAACCGTTGTT
CGACTTTACGCAAACAGATGGGTCTGCTTCTCCACCTCCTGCTCCAAAACATCACCATGC
CTCGAAGGTGGATAGGAAAAAGGCTATTAGAAGTGCAAGTACTTCCGCAATTATGGAAA
CAAAAAGAAACAACAAGAAAGCCGAGGAAAATCAATTTCAAAGCAGCAAAATATTGGG
AAATCCCACGGCTGCACCAAGGAAAAGAGGTAGACCGGTAGGATCTACGAGGGGAAGT
AGGCGGAAGTTAGGTGTCAATTTACAACGTTCTCAAAGTGATATGGGATTTCCTAGACCG
GCGATACCGAATTCTTCAATATCGACAACGCAACTTCCCTCTATTAGATCCACCATGGGA
CCACAATCCCCTACATTGGGTATTCTGGAAGAAGAAAGGCACGATTCTCGACAGCAGCA
GCCGCAACAAAATAATTCTGCACAGTTCAAAGAAATTGATCTTGAGGACGGCTTATCAA
GCGATGTGGAACCTTCACAACAATTACAACAAGTTTTTAATCAAAATACTGGATTTGTAC
CCCAACAACAATCTTCCTTGATACAGACACAGCAAACAGAATCAATGGCCACGTCCGTA
TCTTCCTCTCCTTCATTACCTACGTCACCGGGCGATTTTGCCGATAGTAATCCATTTGAAG
AGCGATTTCCCGGTGGTGGAACATCTCCTATTATTTCCATGATCCCGCGTTATCCTGTAAC
TTCAAGGCCTCAAACATCGGATATTAATGATAAAGTTAACAAATACCTTTCAAAATTGGT
TGATTATTTTATTTCCAATGAAATGAAGTCAAATAAGTCCCTACCACAAGTGTTATTGCA
CCCACCTCCACACAGCGCTCCCTATATAGATGCTCCAATCGATCCAGAATTACATACTGC
CTTCCATTGGGCTTGTTCTATGGGTAATTTACCAATTGCTGAGGCGTTGTACGAAGCCGG
AACAAGTATCAGATCGACAAATTCTCAAGGCCAAACTCCATTGATGAGAAGTTCCTTATT
CCACAATTCATACACTAGAAGAACTTTCCCTAGAATTTTCCAGCTACTGCACGAGACCGT
ATTTGATATCGATTCGCAATCACAAACAGTAATTCACCATATTGTGAAACGAAAATCAAC
AACACCTTCTGCAGTTTATTATCTTGATGTTGTGCTATCTAAGATCAAGGATTTTTCCCCA
CAGTATAGAATTGAATTACTTTTAAACACACAAGACAAAAATGGCGATACCGCACTTCAT
ATTGCTTCTAAAAATGGAGATGTTGTTTTTTTTAATACACTGGTCAAAATGGGTGCATTA
ACTACTATTTCCAATAAGGAAGGATTAACCGCCAATGAAATAATGAATCAACAATATGA
GCAAATGATGATACAAAATGGTACAAATCAACATGTCAATTCTTCAAACACGGACTTGA
ATATCCACGTTAATACAAACAACATTGAAACGAAAAATGATGTTAATTCAATGGTAATC
ATGTCGCCTGTTTCTCCTTCGGATTACATAACCTATCCATCTCAAATTGCCACCAATATAT
CAAGAAATATTCCAAATGTAGTGAATTCTATGAAGCAAATGGCTAGCATATACAACGAT
CTTCATGAACAGCATGACAACGAAATAAAAAGTTTGCAAAAAACTTTAAAAAGCATTTC
TAAGACGAAAATACAGGTAAGCCTAAAAACTTTAGAGGTATTGAAAGAGAGCAGTAAA
GATGAAAACGGCGAAGCTCAGACTAATGATGACTTCGAAATTTTATCTCGTCTACAAGA
ACAAAATACTAAGAAATTGAGAAAAAGGCTCATACGATACAAACGGTTGATAAAACAA
AAGCTGGAATACAGGCAAACGGTTTTATTGAACAAATTAATAGAAGATGAAACTCAGGC
TACCACCAATAACACAGTTGAGAAAGATAATAATACGCTGGAAAGGTTGGAATTGGCTC
AAGAACTAACGATGTTGCAATTACAAAGGAAAAACAAATTGAGTTCCTTGGTGAAGAAA
TTTGAAGACAATGCCAAGATTCATAAATATAGACGGATTATCAGGGAAGGTACGGAAAT
GAATATTGAAGAAGTAGATAGTTCGCTGGATGTAATACTACAGACATTGATAGCCAACA
ATAATAAAAATAAGGGCGCAGAACAGATCATCACAATCTCAAACGCGAATAGTCATGCA
>SEQ ID No: 44 Thioredoxin (TRXA):
agcgataaaattattcacctgactgacgacagttttgacacggatgtactcaaagcggacggggcgatcctcgtcgatttctgggcagagtg
gtgcggtccgtgcaaaatgatcgccccgattctggatgaaatcgctgacgaatatcagggcaaactgaccgttgcaaaactgaacatcgatc
aaaaccctggcactgcgccgaaatatggcatccgtggtatcccgactctgctgctgttcaaaaacggtgaagtggcggcaaccaaagtgggt
gcactgtctaaaggtcagttgaaagagttcctcgacgctaacctggcc
>SEQ ID No: 45 scFV S9.6 GB1 fusion:
GACATAGTTATGACTCAAACCCCGCTTTCCCTCCCAGTCTCACTGGGGGATCAAGCGTCC
ATCTCATGCCGCTCTTCACAGAGTATTGTGCATTCTAACGGTAACACATACCTGGAATGG
TATTTGCAAAAGCCAGGTCAAAGCCCAAAGCTTCTCATCTATAAGGTTTCAAATAGGTTT
TCTGGCGTCCCAGATCGATTCTCCGGGAGTGGGTCTGGTACTGATTTTACTCTTAAGATAT
CAAGAGTCGAGGCCGAGGACTTGGGGGTCTATTACTGTTTCCAAGGGAGCCACGTTCCAT
ATACTTTTGGGGGTGGGACAAAACTGGAAATAAAACGAGGGGGCGGAGGGTCCGGAGG
AGGGGGGAGTGGCGGAGGAGGGTCAGGTGGCGGAGGATCCCAGGTGCAGTTGCAACAG
TCAGGTCCAGAATTGGTTAAACCTGGCGCGTCTGTAAAAATGTCCTGTAAAGCGTCCGGA
TACACGTTTACGAGTTACGTTATGCACTGGGTGAAACAGAAACCGGGGCAGGGCCTGGA
ATGGATCGGGTTTATCAACTTaTACAACGATGGAACAAAGTACAATGAAAAGTTTAAAGG
CAAAGCCACGTTGACTTCAGATAAAAGCTCATCAACTGCATATATGGAGCTGTCATCTCT
TACTTCCAAGGATAGCGCGGTTTATTACTGTGCTCGGGATTATTATGGAAGCAGATGGTT
TGACTATTGGGGACAAGGGACGACATTGACTGTATCTAGCGGTGGAGGTCGGACCGAAG
AGTACAAGCTTATCCTGAACGGTAAAACCCTGAAAGGTGAAACCACCACCGAAGCTGTT
GACGCTGCTACCGCGGAAAAAGTTTTCAAACAGTACGCTAACGACAACGGTGTTGACGG
TGAATGGACCTACGACGACGCTACCAAAACCTTCACGGTAACCGAAGGTGGTGGTAGCG
GTGGTGGTACTAGTCCCAAGAAGAAGCGCAAGGTG
>SEQ ID No: 46 SS07D
GCTACAGTGAAATTTAAGTATAAGGGGGAGGAGAAGGAAGTGGATATCTCCAAGATCAA
GAAGGTGTGGCGCGTAGGGAAAATGATTTCTTTTACTTATGACGAGGGTGGGGGGAAGA
CCGGACGGGGAGCCGTGTCAGAGAAAGACGCCCCCAAGGAGCTCCTGCAGATGCTCGAG
AAGCAGAAAAAA
>SEQ ID No: 47 ADARI
AGCCTTGGAACAGGAAATCGGTGTGTCAAGGGGGACTCATTGAGCCTCAAAGGGGAGAC
AGTAAATGATTGTCACGCGGAAATCATAAGTCGACGGGGCTTCATTCGATTTCTCTACAG
CGAATTGATGAAATACAACTCTCAGACGGCAAAAGATAGCATATTCGAACCTGCGAAAG
GGGGGGAGAAGCTCCAAATCAAGAAGACCGTCAGTTTTCACCTTTATATCAGTACCGCA
CCCTGCGGTGACGGCGCGCTTTTCGACAAGAGTTGTTCAGACCGCGCAATGGAATCCACG
GAAAGCAGACATTATCCAGTCTTTGAGAATCCGAAACAGGGCAAACTCCGGACAAAAGT
CGAAAATGGTCAGGGCACGATCCCCGTTGAGTCTTCAGATATCGTTCCCACCTGGGACGG
GATTAGACTCGGAGAGAGGCTCCGGACGATGAGCTGTTCAGATAAGATCCTGCGATGGA
ATGTCCTGGGCTTGCAAGGCGCGCTGTTGACACACTTTCTTCAGCCAATTTACCTCAAAT
CAGTCACTCTCGGCTACCTCTTTTCACAAGGGCATCTCACCCGGGCCATTTGTTGTCGCGT
GACAAGGGACGGTTCCGCTTTTGAGGACGGGCTTCGCCATCCCTTCATAGTAAATCACCC
CAAGGTCGGACGAGTCTCAATTTACGACTCCAAACGGCAATCAGGAAAGACTAAAGAAA
CGTCTGTCAACTGGTGTCTGGCTGATGGCTACGATCTTGAAATACTTGACGGGACCCGAG
GAACCGTCGACGGCCCCAGGAACGAGCTTAGCAGGGTAAGTAAGAAAAATATATTCCTC
CTCTTCAAGAAACTTTGTTCATTTCGATATAGGCGCGACCTGTTGCGACTGAGCTACGGC
GAGGCCAAGAAGGCGGCGCGCGACTACGAGACCGCCAAGAATTATTTCAAAAAGGGAC
TCAAGGATATGGGCTATGGAAATTGGATTTCCAAACCGCAAGAGGAAAAGAATTTC
>SEQ ID No: 48 ADAR2
cagctgcatttaccgcaggttttagctgacgctgtctcacgcctggtcctgggtaagtttggtgacctgaccgacaacttctcctcccctc
acgctcgcagaaaagtgctggctggagtcgtcatgacaacaggcacagatgttaaagatgccaaggtgataagtgtttctacaggaacaaa
atgtattaatggtgaatacatgagtgatcgtggccttgcattaaatgactgccatgcagaaataatatctcggagatccttgctcagattt
ctttatacacaacttgagctttacttaaataacaaagatgatcaaaaaagatccatctttcagaaatcagagcgaggggggtttaggctg
aaggagaatgtccagtttcatctAtacatcagcacctctccctgtggagatgccagaatcttctcaccacatgagccaatcctggaagaac
cagcagatagacacccaaatcgtaaagcaagaggacagctacggaccaaaatagagtctggtCaggggacgattccagtgcgctccaatgc
gagcatccaaacgtgggacggggtgctgcaaggggagcggctgctcaccatgtcctgcagtgacaagattgcacgctggaacgtggtgggc
atccagggatcActgctcagcattttcgtggagcccatttacttctcgagcatcatcctgggcagcctttaccacggggaccacctttcca
gggccatgtaccagcggatctccaacatagaggacctgccacctctctacaccctcaacaagcctttgctcagtggcatcagcaatgcaga
agcacggcagccagggaaggcccccaacttcagtgtcaactggacggtaggcgactccgctattgaggtcatcaacgccacgactgggaag
gatgagctgggccgcgcgtcccgcctgtgtaagcacgcgttgtactgtcgctggatgcgtgtgcacggcaaggttccctcccacttactac
gctccaagattaccaagcccaacgtgtaccatgagtccaagctggcggcaaaggagtaccaggccgccaaggcgcgtctgttcacagcctt
catcaaggcggggctgggggcctgggtggagaagcccaccgagcaggaccagttctcactcacg
>SEQ ID No: 49 rat apolipoprotein B mRNA editing enzyme, catalytic polypeptide-like 1
(rAPOBEC):
agcagtgaaaccggaccagtggcagtggacccaaccctgaggagacggattgagccccatgaatttgaagtgttctttgacccaagggagct
gaggaaggagacatgcctgctgtacgagatcaagtggggcacaagccacaagatctggcgccacagctccaagaacaccacaaagcacgtgg
aagtgaatttcatcgagaagtttacctccgagcggcacttctgcccctctaccagctgttccatcacatggtttctgtcttggagcccttgc
ggcgagtgttccaaggccatcaccgagttcctgtctcagcaccctaacgtgaccctggtcatctacgtggcccggctgtatcaccacatgga
ccagcagaacaggcagggcctgcgcgatctggtgaattctggcgtgaccatccagatcatgacagccccagagtacgactattgctggcgga
acttcgtgaattatccacctggcaaggaggcacactggccaagatacccacccctgtggatgaagctgtatgcactggagctgcacgcagg
aatcctgggcctgcctccatgtctgaatatcctgcggagaaagcagccccagctgacatttttcaccattgctctgcagtcttgtcactat
cagcggctgcctcctcatattctgtgggctacaggcctgaag
>SEQ ID No: 50 Activation-induced cytidine deaminase (AID):
GACAGTCTGTTGATGAATCGCCGCAAATTTTTGTATCAGTTCAAAAATGTGCGTTGGGCC
AAGGGCCGCCGCGAAACATACCTCTGTTATGTAGTGAAACGTCGTGATAGCGCAACATC
ATTCAGCCTGGACTTCGGATACCTGCGCAACAAAAACGGTTGCCACGTGGAGTTGCTGTT
CCTGCGTTACATCTCAGATTGGGATCTTGATCCGGGCCGTTGTTACCGTGTGACCTGGTTC
ACATCGTGGTCCCCGTGCTATGATTGCGCCCGTCACGTTGCGGATTTTTTACGTGGTAACC
CGAATTTGAGCCTGCGCATTTTTACAGCGCGTCTGTATTTTTGCGAAGACCGTAAGGCGG
AACCGGAAGGTCTGCGTCGTTTGCATCGCGCGGGgGTACAGATCGCTATCATGACCTTTA
AAGATTATTTTTACTGCTGGAACACCTTTGTGGAAAACCATGAACGCACGTTTAAAGCGT
GGGAAGGCCTCCACGAAAATTCGGTACGTCTGTCgCGTCAGCTGCGCCGTATCTTACTGC
CGCTGTATGAGGTCGATGATCTGCGCGACGCCTTTCGTACcTTGGGCCTG

1. A method for modifying a target locus in a genome in a cell, comprising
introducing into the cell: a Cas9 nickase (nCas9), a reverse transcriptase (RT), and an extended guide RNA (gRNA), wherein the extended gRNA comprises a guide RNA and an RNA template for the RT;
wherein the extended gRNA binds to a DNA strand at the target locus in the genome; and
wherein the RNA template comprises a desired mutation to be introduced into the target locus,
thereby modifying the target locus in the genome.

2. The method of claim 1, wherein the method does not induce double-stranded DNA breaks.

3. The method of claim 1, wherein the Cas9 nickase nicks a DNA strand that is not bound by the extended gRNA.

4. The method of claim 1, wherein the Cas9 nickase introduces two nicks onto the DNA strand that is not bound by the extended gRNA.

5. The method of claim 1, wherein the RNA template hybridizes to the DNA strand that is not bound by the extended gRNA to form a RNA/DNA hybrid.

6. The method of claim 1, wherein the reverse transcriptase primes from the RNA/DNA hybrid and extends the DNA strand based on the RNA template in the extended gRNA to introduce the desired mutation into the target locus.

7. The method of claim 1, wherein the desired mutation is introduced upstream of a nick introduced by the Cas9 nickase.

8. The method of claim 7, wherein the reverse transcriptase has preserved 3′ to 5′ exonuclease activity to enable the desired mutation to be introduced upstream of the 3′ nick.

9. The method of claim 1, wherein the desired mutation is introduced downstream of a nick introduced by the Cas9 nickase.

10. The method of claim 1, wherein the reverse transcriptase is an error prone reverse transcriptase which diversifies a DNA region of interest.

11. The method of claim 1, wherein the reverse transcriptase is a human immunodeficiency virus reverse transcriptase (HIV RT).

12. The method of claim 1, wherein the reverse transcriptase is fused to the N-terminus or the C-terminus of the Cas9 nickase.

13. The method of claim 12, wherein the reverse transcriptase is fused to the Cas9 nickase via a linker.

14. The method of claim 13, wherein the linker is a Gly-Ser rich linker or an XTEN linker.

15. The method of claim 1, wherein the RNA template is fused to either the 5′ end or the 3′ end of the guide RNA.

16. The method of claim 15, wherein the RNA template is fused to the guide RNA via a linker.

17. The method of claim 1, wherein the desired mutation comprises a point mutation, an insertion, or a deletion.

18. The method of claim 1, wherein a DNA repair protein is recruited during extension of the DNA strand at the target locus.

19. The method of claim 1, wherein the extended gRNA further comprises sequences that block exonuclease activity.

20. The method of claim 1, wherein the cell is a mammalian cell.


SEQUENCE LISTING (50 sequences)

SEQ ID NO: 1 - DNA, 7386 bp, Artificial Sequence
  Description of Artificial Sequence Synthetic polynucleotide
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgctatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggttctggaaaacggacagcggacggtagcgagtttgagagtccgaagaaaaagaggaaagtagagggtggttctgccggtggctccggttctggctccagcggtggcagctctggtgcgtccggcacgggtactgcgggtggcactggcagcggttccggtactggctctggccccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactaggcgggggttctgggggaggatcaggtggtgggtccgggggaggaagcgggggtggctctgggggtggatcaccgattagcccgattgaaaccgttccggttaaactgaaaccgggtatggatggtccgaaagttaaacagtggcctctgaccgaagaaaaaatcaaagcactggttgaaatctgcaccgagatggaaaaagaaggcaaaattagcaaaatcggtccggaaaatccgtataatacaccggtttttgccattaagaaaaaagatagcaccaaatggcgcaaactggtggattttcgtgaactgaataaacgcacccaggatttttgggaagttcagctgggtattccgcatccggcaggtctgaaacagaaaaaaagcgttaccgttctggatgttggtgatgcatattttagcgttccgctggataaagatttccgtaaatataccgcatttaccatcccgagcattaataacgaaacaccgggtattcgctatcagtataatgttctgccgcagggttggaaaggtagtccggcaatttttcagtgtagcatgaccaaaattctggaaccgtttcgtaaacagaatccggatattgtgatctaccagtatatggatgatctgtatgttggtagcgatctggaaattggtcagcatcgtaccaaaattgaagaactgcgtcagcatctgctgcgttggggttttaccacaccggataaaaaacatcagaaagaaccgccttttctgtggatgggttatgaactgcatccggataaatggaccgttcagccgattgttctgccggaaaaagatagctggaccgttaatgatattcagaaactggtgggtaaactgaattgggcaagccagatttatgccggtattaaagttcgtcagctgtgtaaactgctgcgtggcaccaaagcactgaccgaagttgttccgctgacagaagaagcagaactggaactggcagaaaatcgtgaaattctgaaagaaccggttcacggcgtttattatgatccgagcaaagatctgattgccgaaattcagaaacagggtcagggtcagtggacctatcagatttatcaagaaccgtttaaaaacctgaaaaccggcaaatatgcacgtatgaaaggtgcacataccaacgatgttaaacagctgaccgaagcagttcagaaaattgcaaccgaaagcattgtgatttggggtaaaaccccgaaattcaaactgccgattcagaaagaaacctgggaagcatggtggaccgaatattggcaggcaacctggattccggaatgggaatttgttaatacccctccgctggttaaactgtggtatcagctggaaaaagaaccgattattggtgccgaaaccttttga

SEQ ID NO: 2 - DNA, 7388 bp, Artificial Sequence
  Description of Artificial Sequence Synthetic polynucleotide
atgcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactaggcgggggttctgggggaggatcaggtggtgggtccgggggaggaagcgggggtggctctgggggtggatcaccgattagcccgattgaaaccgttccggttaaactgaaaccgggtatggatggtccgaaagttaaacagtggcctctgaccgaagaaaaaatcaaagcactggttgaaatctgcaccgagatggaaaaagaaggcaaaattagcaaaatcggtccggaaaatccgtataatacaccggtttttgccattaagaaaaaagatagcaccaaatggcgcaaactggtggattttcgtgaactgaataaacgcacccaggatttttgggaagttcagctgggtattccgcatccggcaggtctgaaacagaaaaaaagcgttaccgttctggatgttggtgatgcatattttagcgttccgctggataaagatttccgtaaatataccgcatttaccatcccgagcattaataacgaaacaccgggtattcgctatcagtataatgttctgccgcagggttggaaaggtagtccggcaatttttcagtgtagcatgaccaaaattctggaaccgtttcgtaaacagaatccggatattgtgatctaccagtatatggatgatctgtatgttggtagcgatctggaaattggtcagcatcgtaccaaaattgaagaactgcgtcagcatctgctgcgttggggttttaccacaccggataaaaaacatcagaaagaaccgccttttctgtggatgggttatgaactgcatccggataaatggaccgttcagccgattgttctgccggaaaaagatagctggaccgttaatgatattcagaaactggtgggtaaactgaattgggcaagccagatttatgccggtattaaagttcgtcagctgtgtaaactgctgcgtggcaccaaagcactgaccgaagttgttccgctgacagaagaagcagaactggaactggcagaaaatcgtgaaattctgaaagaaccggttcacggcgtttattatgatccgagcaaagatctgattgccgaaattcagaaacagggtcagggtcagtggacctatcagatttatcaagaaccgtttaaaaacctgaaaaccggcaaatatgcacgtatgaaaggtgcacataccaacgatgttaaacagctgaccgaagcagttcagaaaattgcaaccgaaagcattgtgatttggggtaaaaccccgaaattcaaactgccgattcagaaagaaacctgggaagcatggtggaccgaatattggcaggcaacctggattccggaatgggaatttgttaatacccctccgctggttaaactgtggtatcagctggaaaaagaaccgattattggtgccgaaaccttttgaggtggttctgccggtggctccggttctggctccagcggtggcagctctggtgcgtccggcacgggtactgcgggtggcactggcagcggttccggtactggctctggcgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgctatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgggttctggaaaacggacagcggacggtagcgagtttgagagtccgaagaaaaagaggaaagtagatga

SEQ ID NO: 3 - DNA, 156 bp, Artificial Sequence
  Description of Artificial Sequence Synthetic polynucleotide
gagtccgagcagaagaagaagttttagagctagaaatagcaagttaaaataaggctagtccgttatcaacttgaaaaagtggcaccgagtcggtgccgccaccggttgatgtgatgggagcccttcctcttctgctcggactcaggcccttcctcc

SEQ ID NO: 4 - DNA, 153 bp, Artificial Sequence
  Description of Artificial Sequence Synthetic polynucleotide
gagtccgagcagaagaagaagttttagagctagaaatagcaagttaaaataaggctagtccgttatcaacttgaaaaagtggcaccgagtcggtgccgccaccggttgatgtgatgggagcccttcttctgctcggactcaggcccttcctcc

SEQ ID NO: 5 - DNA, 199 bp, Artificial Sequence
  Description of Artificial Sequence Synthetic polynucleotide
gagtccgagcagaagaagaagttttagagctagaaatagcaagttaaaataaggctagtccgttatcaacttgaaaaagtggcaccgagtcggtgctctctccgcttatcttctctatttcctttattccgtccctccacgccaccggttgatgtgatgggagcccttcctcttctgctcggactcaggcccttcctcc

SEQ ID NO: 6 - DNA, 196 bp, Artificial Sequence
  Description of Artificial Sequence Synthetic polynucleotide
gagtccgagcagaagaagaagttttagagctagaaatagcaagttaaaataaggctagtccgttatcaacttgaaaaagtggcaccgagtcggtgctctctccgcttatcttctctatttcctttattccgtccctccacgccaccggttgatgtgatgggagcccttcttctgctcggactcaggcccttcctcc

SEQ ID NO: 7 - DNA, 2136 bp, Artificial Sequence
  Description of Artificial Sequence Synthetic polynucleotide
gctactggacaggatcgagtggttgctctcgtggacatggactgtttttttgttcaagtggagcagcggcaaaatcctcatttgaggaataaaccttgtgcagtcgtacagtacaaatcatggaagggtggtggaataattgcagtgagttatgaagctcgtgcatttggagtcactagaagtatgtgggcagatgatgctaagaagttatgtccagatcttctactggcacaagttcgtgagtcccgtgggaaagctaacctcaccaagtaccgggaagccagtgttgaagtgatggagataatgtctcgttttgctgtgattgaacgtgccagcattgatgaggcttacgtagatctgaccagtgccgtacaagagagactacaaaagctacaaggtcagcctatctcggcagacttgttgccaagcacttacattgaagggttgccccaaggccctacaacggcagaagagactgttcagaaagaggggatgcgaaaacaaggcttatttcaatggctcgattctcttcagattgataacctcacctctccagacctgcagctcaccgtgggagcagtgattgtggaggaaatgagagcagccatagagagggagactggttttcagtgttcagctggaatttcacacaataaggtcctggcaaaactggcctgtggactaaacaagcccaaccgccaaaccctggtttcacatgggtcagtcccacagctcttcagccaaatgcccattcgcaaaatccgtagtcttggaggaaagctaggggcctctgtcattgagattctagggatagaatacatgggtgaactgacccagttcactgaatcccagctccagagtcattttggggagaagaatgggtcttggctatatgccatgtgccgagggattgaacatgatccagttaaacccaggcaactacccaaaaccattggctgtagtaagaacttcccaggaaaaacagctcttgctactcgggaacaggtacaatggtggctgttgcaattagcccaggaactagaggagagactgactaaagaccgaaatgataatgacagggtagccacccagctggttgtgagcattcgcgtacaaggagacaaacgcctcagcagcctgcgccgctgctgtgcccttacccgctatgatgctcacaagatgagccatgatgcatttactgtcatcaagaactgtaatacttctggaatccagacagaatggtctcctcctctcacaatgcttttcctctgtgctacaaaattttctgcctctgccccttcatcttctacagacatcaccagcttcttgagcagtgacccaagttctctgccaaaggtgccagttaccagctcagaagctaagacccagggaagtggcccagcggtgacagccactaagaaagcaaccacgtctctggaatcattcttccaaaaagctgcagaaaggcagaaagttaaagaagcttcgctttcatctcttactgctcccactcaggctcccatgagcaattcaccatccaagccctcattaccttttcaaaccagtcaaagtacaggaactgagcccttctttaagcagaaaagtctgcttctaaagcagaaacagcttaataattcttcagtttcttccccccaacaaaacccatggtccaactgtaaagcattaccaaactctttaccaacagagtatccagggtgtgtccctgtttgtgaaggggtgtcgaagctagaagaatcctctaaagcaactcctgcagagatggatttggcccacaacagccaaagcatgcacgcctcttcagcttccaaatctgtgctggaggtgactcagaaagcaaccccaaatccaagtcttctagctgctgaggaccaagtgccctgtgagaagtgtggctccctggtaccggtatgggatatgccagaacacatggactatcattttgcattggagttgcagaaatcctttttgcagccccactcttcaaacccccaggttgtttctgccgtatctcatcaaggcaaaagaaatcccaagagccctttggcctgcactaataaacgccccaggcctgagggcatgcaaacattggaatcattttttaagccattaacacat

SEQ ID NO: 8 - DNA, 1065 bp, Artificial Sequence
  Description of Artificial Sequence Synthetic polynucleotide
acatcctgggtcttgcacgtagacctcgatcaattccttgccagcgtggagttgcggcgcagacccgacctgagaggtctcccggtaatcgtagggggatcaggcgatcccaccgagccgcgcaaagttgtcacgtgtgctagttacgaggcgcgcgagttcggtgtccatgctggcatgccgctgagggccgcggctcgaaggtgcccagacgccacatttcttccttctgatcccgcagcatacgatgaagccagcgagcaggtaatggggttgctgagggacttggggcaccctttggaagtatgggggtgggatgaggcgtacttgggtgccgacttggagcctgacgcagatccggtggaactcgccgaaaggataagaactgtcgttgccgctgaaacggggctttcctgttctgtaggaatatccgacaacaagcaaagagcaaaggtggcaactgggtttgcaaaaccagcgggtatctacgtgcttactgaagcaaattggatgaccgtaatgggcgatagacccccggatgcgctctggggtatcgggcctaaaacgaccaagaagttggcggcaatgggcataacaacagtcgcggatctcgcggccaccgacgcaagtgttctcactgcggcgttcggtcctagtaccggactgtggatattgctcctcgccaaaggagggggagatactgaggtgtcaagtgagccgtggatacccagatcccgctcacatgtagtgacttttccgcaggacctcaccgaccggcgggaaatcgattccgccgtccgcgaccttgcacttcagacacttactgagatcgttgagcaagggcgcaccgttactagagttgctgtcacggtgcggacatctacattttacacgcgaaccaagatacgaaagctgccaacaccgggtactgacgctgatcaaatagtggcgaccgcactggcagtcttggaccaattcgaattggatcgacctgtccgactccttggcgttcgactcgagcttgcaatggatgatgttgcggcaccgaccgttggtaccgggaca

SEQ ID NO: 9 - DNA, 3072 bp, Artificial Sequence
  Description of Artificial Sequence Synthetic polynucleotide
cccattagtcctattgagactgtaccagtaaaattaaagccaggaatggatggcccaaaagttaaacaatggccattgacagaagaaaaaataaaagcattagtagaaatttgcacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactaggcgggggttctgggggaggatcaggtggtgggtccgggggaggaagcgggggtggctctgggggtggatcaccgattagcccgattgaaaccgttccggttaaactgaaaccgggtatggatggtccgaaagttaaacagtggcctctgaccgaagaaaaaatcaaagcactggttgaaatctgcaccgagatggaaaaagaaggcaaaattagcaaaatcggtccggaaaatccgtataatacaccggtttttgccattaagaaaaaagatagcaccaaatggcgcaaactggtggattttcgtgaactgaataaacgcacccaggatttttgggaagttcagctgggtattccgcatccggcaggtctgaaacagaaaaaaagcgttaccgttctggatgttggtgatgcatattttagcgttccgctggataaagatttccgtaaatataccgcatttaccatcccgagcattaataacgaaacaccgggtattcgctatcagtataatgttctgccgcagggttggaaaggtagtccggcaatttttcagtgtagcatgaccaaaattctggaaccgtttcgtaaacagaatccggatattgtgatctaccagtatatggatgatctgtatgttggtagcgatctggaaattggtcagcatcgtaccaaaattgaagaactgcgtcagcatctgctgcgttggggttttaccacaccggataaaaaacatcagaaagaaccgccttttctgtggatgggttatgaactgcatccggataaatggaccgttcagccgattgttctgccggaaaaagatagctggaccgttaatgatattcagaaactggtgggtaaactgaattgggcaagccagatttatgccggtattaaagttcgtcagctgtgtaaactgctgcgtggcaccaaagcactgaccgaagttgttccgctgacagaagaagcagaactggaactggcagaaaatcgtgaaattctgaaagaaccggttcacggcgtttattatgatccgagcaaagatctgattgccgaaattcagaaacagggtcagggtcagtggacctatcagatttatcaagaaccgtttaaaaacctgaaaaccggcaaatatgcacgtatgaaaggtgcacataccaacgatgttaaacagctgaccgaagcagttcagaaaattgcaaccgaaagcattgtgatttggggtaaaaccccgaaattcaaactgccgattcagaaagaaacctgggaagcatggtggaccgaatattggcaggcaacctggattccggaatgggaatttgttaatacccctccgctggttaaactgtggtatcagctggaaaaagaaccgattattggtgccgaaaccttt

SEQ ID NO: 10 - DNA, 2031 bp, Artificial Sequence
  Description of Artificial Sequence Synthetic polynucleotide
actgtctcccttcaagatgaacacagactgtttgacatccctgttactacatccctccctgacgtatggttgcaggatttccctcaagcgtgggccgagacaggtggtcttggtcgggcaaaatgtcaggctccaataatcattgatctgaagcccacagccgttccggttagtataaaacagtacccaatgagtctcgaggcacatatggggattcgacaacacattataaaatttctggaattgggggtcttgagaccgtgtcgcagtccttggaacacgcccttgctgccggtcaagaaacctggtacccaggattaccgcccggtgcaagatcttcgcgaaataaataagcgcactgttgacatccatccaactgtccccaatccatacaatctgctttccacattgaagccggattatagctggtacaccgtcctggaccttaaggatgccttcttttgtctccctctcgctccacagtcccaggagctttttgcgttcgagtggaaggaccccgagcgagggatttctgggcagttgacgtggacccgcctgccgcagggatttaagaacagccccacactctttgatgaagccctccacagagacctgactgatttccgaacgcagcatccggaggtgacactgctgcaatatgtggatgatctcctccttgctgcgccaactaaaaaagcgtgcacgcagggtacgagacatctcttgcaggagcttggagagaaaggctatagggcgagcgccaaaaaagctcaaatctgccagacgaaggtcacctaccttggatacatattgtccgaagggaagaggtggctcactcccgggaggatagaaacagtagctcgcattcctccgccccgcaatccaagggaggtgagagaattccttgggacagctggtttttgtcgattgtggatccccggctttgccgagttggccgctccgctgtatgcgcttacaaaagagagcacgcccttcacctggcaaactgaacatcagctcgcctttgaagcgcttaaaaaagcactgctctccgcaccggcgttgggcctgccggacacgtccaaacctttcactctcttcctggacgagcggcaaggaatagctaaaggagtgctgacccagaaacttgggccatggaagaggcctgtcgcatatctgtctaagaagctcgatcccgttgcagcgggatggcccccatgcctgcggataatggcggcaacagctatgcttgtaaaggacagcgcaaaacttactttggggcaaccactgacagtcataactcctcatacacttgaagcgatcgtgcgacaaccaccagaccgctggattacaaatgctagactcacccattaccaggctctgttgttggacacagacagagtgcaatttggtccgcccgtcacccttaatcctgctaccctccttccggtgccagaaaatcaaccctccccacacgattgccgacaggttctcgctgagacacacgggacccgcgaagacctgaaagatcaggaactgcctgatgccgatcatacgtggtacacagatgggagcagttacctggattcaggaacaagaagggcaggagccgcagtcgtggacggtcataatacgatctgggcccagtcattgccccctgggactagcgcccagaaggcggagctcattgctctgaccaaagcgttggaactttccaagggtaagaaagctaacatttacacggacagtcgctatgcttttgctactgctcacacccatggaagtatatacgagcggcgaggactgttgacttcagagggtaaagaaatcaaaaataaggccgaaataattgcgctcttgaaggctctgttcctgccgcaagaagtggctatcatccattgtccaggtcatcagaaggggcaagacccggtcgcagttggtaaccggcaagcagatagagtagcgagacaagccgcaatggcagaagttctgaccttggcgactgaacccgacaacacttcacatataact

SEQ ID NO: 11 - DNA, 2007 bp, Artificial Sequence
  Description of Artificial Sequence Synthetic polynucleotide
gtgttgaacctcgaggaggaatatcgactccatgaaaagcccgttccgtccagtattgacccctcctggctccaactgtttcctacagtatgggcagagcgagcggggatgggcctggctaatcaagtcccgccagttgttgttgagctccgctctggagcatctccggtagcggtccgacagtacccaatgagtaaggaagctcgggaggggatccgcccccacattcaacgctttctggatctgggcgtactcgtaccttgccagtcaccatggaatacaccgctcctgccagtaaaaaagcctggcacaaatgactatagacctgtgcaggacctgagggagatcaacaaacgggtgcaagacatacatcctacagtccctaacccctacaacttgctgagcagccttccgcccagtcacacatggtactctgtcctggaccttaaagacgcttttttttgtttgaagttgcatccaaattctcaacccttgttcgcattcgagtggagggacccagaaaagggaaacacaggccagctgacctggactagactgccccaaggattcaaaaacagcccaacgttgttcgatgaagctttgcacagagatctcgcaccgttccgagctctcaatcctcaagtcgtactgctgcagtacgtagacgatcttttggtagctgcgccgacttatcgggattgtaaagaaggcactcagaagctccttcaagaactgtcaaaactcggctatagggtctcagctaaaaaagctcagctgtgccagaaagaggtcacatatctcggttacttgcttaaggaagggaagcgatggcttacgccggcccgaaaagcgaccgttatgaagataccccctccgactacgccccgccaagtccgggagttcctgggaacagccggtttctgccggctttggattcccggattcgctagtttggctgcgcccctgtatcccctcacgaaagaatctattccttttatttggactgaggaacaccaaaaggcctttgatagaataaaagaagccttgttgtcagcgcccgcactggccctgcctgacctgacgaaaccatttacactctacgtcgatgagcgcgctggtgtggcacggggagtactgactcaaacgctcggtccatggcgccgaccagtcgcgtacctctctaagaaacttgatccagtcgcatcaggatggccgacatgccttaaagcagtagctgccgttgccctgctcttgaaggacgcagacaaactcacactcggccagaatgtgacagtcatcgcgagtcactccctggagtccatcgtaagacaacctccagaccgctggatgacaaacgcacgcatgacacattaccaatctctgcttctgaatgagcgggtcagctttgcgccgcccgctgtacttaatcccgcgacccttcttcctgtggaaagtgaggcgacacccgttcacaggtgctcagagattcttgctgaagaaacaggcacccggagagaccttaaagatcaacccctgccgggtgttccggcgtggtataccgacggtagcagtttcattgcggaagggaagcgacgagccggcgctgcgatcgttgatgggaagaggactgtgtgggcttcctccctgcctgaagggacatctgctcaaaaggctgagctcgtcgcccttacacaagcccttcgattggcggaaggcaaggacataaacatctatacagattcccggtatgcctttgctactgcacatatacatggtgcaatttacaaacagaggggcctcttgacaagtgctggtaaggatatcaaaaacaaggaggaaatcctggcgttgttggaggcaattcacctcccaaagcgcgttgcaataatccattgtccgggtcaccaaaaaggcaacgacccagtggcgacagggaacagacgggctgacgaggcagcgaagcaagctgcgctgtccacccgcgtgttggcagagacaacaaaaccg

SEQ ID NO: 12 - DNA, 2007 bp, Artificial Sequence; identical to SEQ ID NO: 11
  Description of Artificial Sequence Synthetic polynucleotide

SEQ ID NO: 13 - DNA, 1941 bp, Artificial Sequence
  Description of Artificial Sequence Synthetic polynucleotide
ctccaagattttccgcaagcttgggccgaaactggcggcttgggacgagcgaagtgccaggttccgattattattgaccttaaacctacagcaatgcctgtttccattaggcagtatccaatgagcaaagaggcacatatgggaattcaaccacatattacccggttcctggagctgggggttttgcggccatgccgatcaccatggaatactccactgcttcctgttaagaagcccggtacccgcgactaccgcccagtgcaggatcttagggaagtgaacaaaaggactatggatattcacccaaccgttcccaacccatataatctgctgagcacactctctcccgaccgaacctggtatacagttctcgatttgaaagatgcgttcttttgcctgcctttggctcctcagagccaagaactctttgcgtttgagtggcgcgatccggaacgcggtatctcagggcagttgacctggacacgccttcctcagggttttaaaaatagcccaacgcttttcgatgaagcgttgcatcgggatcttacagatttcaggacacagcatcccgaggttacattgctgcagtatgtggatgatctgcttctggctgctccgacgaaggaggcctgtattagaggtactaaacaccttctgcgagagcttggcgataaaggttatagggcctctgcgaaaaaagcgcagatctgtcaaacaaaggtcacgtatttgggatatattttgagtgaaggtaaacgatggctcaccccggggcggattgagactgtcgcacacataccacctccacaaaatcctcgggaagtccgcgagttcctcggcaccgcgggattctgtagactttggatcccgggattcgctgaacttgcggcacccctctacgcgctcaccaaggaatctgctcctttcacgtggcaggagaagcaccagtccgcgttcgaggcccttaaggaagctttgctttctgcaccagccctgggcctgcccgatacgagtaaaccctttactctctttatagatgagaagcaggggattgcgaaaggcgtgctgacacaaaagctcgggccgtggaaacgcccggtcgcctacttgtctaagaagcttgacccagtcgctgcaggatggccaccctgcctgaggatcatggcggccactgctatgctcgtcaaggattcagcaaagctcacgctgggtcagcctttgacggtaattactccgcatgcacttgaggcaattgttcggcaaactcctgatagatggatcacgaatgctcgccttacgcattaccaagcactcctgcttgataccgataggattcaatttggaccacctgtcactcttaaccctgcgactctgcttccggcgccagaggatcaacaaagcgctcacgactgtaggcaggtacttgctgaaacccatggaactcgagaggaccttaaggatcaagagctccccgacgcagaccatagctggtacacagacgggtccagttacatagactctggcacacgcagagcaggggctgctgtggtggacggtcatcacattatatgggcccagtcacttcccccggggacatcagcccaaaaggcggagctcatagcattgacaaaagctttggaactgagtgaaggtaaaaaagctaacatttacacggactcacggtatgccttcgccacggcgcacacgcacggctccatatacgagcggcgaggattgctcacatctgagggaaaggaaataaagaataaggccgaaataatagccctgttgaaagctttgtttctccctcgcaaagttgcgattatccattgcccaggccatcagaaaggacaagaccctatcgctactgggaatagacaggccgatcaggttgccagacaggttgccgtggctgaaactcttacactcacgacgaagctt

SEQ ID NO: 14 - DNA, 2007 bp, Artificial Sequence
  Description of Artificial Sequence Synthetic polynucleotide
gttttgaacctcgaagaagagtaccggctgcacgaaaaaccggtcccttcaagcatcgacccttcttggcttcagctcttcccgaccgtttgggcagaaagagctggtatgggcctcgcgaaccaggtacctcccgtagtggtggagttgaggagcggtgcgtcccccgtagctgtgaggcagtatcctatgtctaaagaagcgcgcgaaggtatacgcccccatatccaaaagtttctggacctgggtgtcctcgttccatgtcgctccccgtggaatacccctttgctgccggtaaagaagcctggaactaatgattaccgccccgtccaagatcttcgagagattaataaacgcgtacaggatatccacccaactgtaccaaatccctacaatctcctgagcagtcttcctccttcatacacgtggtattcagtgctcgatcttaaagatgccttcttttgcctgagacttcatcctaatagtcaaccgctctttgcttttgaatggaaagatccagaaaaaggcaacactggtcagctgacgtggacgaggcttcctcagggttttaaaaattcccccaccctcttcgatgaggcgcttcatcgagacctcgctcctttcagagctctgaatccccaagtggtactgcttcagtacgtcgatgatctgttggttgccgctccgacttatgaggactgcaagaagggcacacagaagctcctgcaggaacttagcaaacttggctacagagtgtctgcgaagaaagctcaattgtgtcagagagaggttacatatctgggctaccttttgaaagagggaaaaagatggctgacaccagccaggaaggcaacagtaatgaagattcctgtacccactacgccccggcaagtaagagaatttttgggtaccgcaggattttgcagactgtggatccctggctttgcgtcacttgccgcacccctttacccacttactaaggaatccatcccttttatctggactgaggagcaccagcaggcctttgaccacatcaaaaaagcactgctgagtgcgccagctttggccctgcctgacctgacgaagccatttacgttatacatcgacgagagggctggtgtggcacggggggtgctcacgcaaacgctcggcccttggaggcggccagttgcttaccttagtaagaagcttgacccagttgcgtcaggctggccgacatgcttgaaagccgttgccgcggtcgccctgttgttgaaggacgctgacaagttgacgctggggcaaaatgtcactgtgattgcgtcccactctctcgagagtatcgttcgccaaccccccgacaggtggatgactaacgccagaatgacacactaccagtcacttctcttgaacgaaagggttagcttcgccccacccgccgtcctgaatccggcgactcttcttcctgtggaaagtgaggccacaccagtacatagatgctcagagatacttgccgaagaaacaggaacccggagggacctggaagatcaacctttgccgggcgtaccaacctggtatacagacggatcttcctttattacggaaggcaagcgacgggcgggtgctcctatcgttgatgggaagcggacagtatgggcgagcagccttccagaaggcacttctgctcagaaagcggagttggttgcactcactcaagcgcttagacttgctgaggggaagaatattaatatatatacggattctcgctatgcattcgcgacggcccacatccatggcgcaatctacaagcagcgcggattgctgacctccgctggcaaggatataaagaataaggaggagattctggcgctgcttgaggcgatacatttgccacgcagggtagccataatacattgccccggacaccagaggggctctaatccggtggccactggcaaccgaagagcggacgaggccgctaagcaagcagcactttcaacgcgggtacttgccggtacgaccaaaccc

SEQ ID NO: 15 - DNA, 1950 bp, Artificial Sequence
  Description of Artificial Sequence Synthetic polynucleotide
tcctgccagacgaagaatacattgaacatcgacgagtatttgctgcaatttccggaccaactttgggcctcccttcctactgacattggcaggatgcttgtacctccaattaccataaaaataaaggacaacgcgagccttccgtctattcgacaatacccattgcccaaggataaaaccgagggcctcaggccgctcattagttccctcgaaaatcaggggatccttataaaatgccattctccgtgtaatacaccaatcttccctatcaagaaggctgggcgcgatgaatatagaatgatacacgacctgcgcgctattaataatatagtggctccactgactgctgttgtcgcgtcccccaccacagtgcttagcaacctcgcccctagcctgcattggttcacagtcattgaccttagtaatgcattttttagcgtacctatacacaaggacagtcaatacttgtttgccttcactttcgaggggcaccaatacacttggaccgtccttccccagggtttcattcatagtcccacgctcttttctcaagctctttaccagtcactccataagatcaagtttaaaatctctagcgaaatttgcatttacatggatgacgtactcatagcctcaaaagacagggacacgaatcttaaagatacagcggttatgcttcagcatctggcatccgaggggcacaaggtgtccaaaaagaaattgcagttgtgtcagcaagaggttgtgtaccttggacaactcctgacccctgaaggtcggaaaattcttccagatcgaaaggttacagtcagccaattccagcaacctactacgatccgacaaattcgggcgtttcttggactcgtgggttattgtagacattggatcccagagttctccatacactccaaattcctggagaagcagttgaagaaggacacggcggagccgtttcaattggacgatcagcaggttgaagcattcaacaaacttaaacatgcgataaccaccgcgccagttcttgtggtaccagatcctgccaagccctttcagttatacacgagtcacagcgagcacgcatctattgccgttttgacgcaaaagcatgcaggaagaacaaggccaattgcctttctttcctctaagttcgatgctatcgagtcaggccttcccccgtgtctgaaggcttgcgccagtattcaccgctccttgacccaggctgactccttcatactgggcgcacccctgattatctacacaactcacgctatctgcacactcctccagagggaccgaagccagcttgtaaccgcatctcgatttagcaagtgggaagccgatcttcttagaccggaattgacatttgtggcttgctccgcggtgagccccgcgcacctatacatgcaatcctgtgaaaataatattccaccgcatgactgcgttctcctcacccacacaatctcaaggccgcggccggacttgagtgatctgccaattccggacccggacatgaccctgttcagcgatggatcttataccaccggacgggggggtgcagcagtagtcatgcatcgccccgttacggatgatttcatcataatccaccaacagccgggtggagcctccgcgcaaacagcggaactcctcgctctcgccgcggcgtgccatcttgccacggacaaaacagtcaacatatacactgactcacggtacgcgtatggcgtcgttcacgattttggtcacctctggatgcacaggggattcgtaactagtgccggtacgccgataaaaaatcataaggagatagaatatcttctcaagcaaattatgaagcccaagcaggtatccgttataaaaattgaagcacacaccaaaggcgtaagcatggaggttcggggcaatgcagctgcagatgaggcggctaaaaacgctgtgtttttggtacagcgg

SEQ ID NO: 16 - DNA, 1380 bp, Artificial Sequence
  Description of Artificial Sequence Synthetic polynucleotide
agcctggacatccagagcctggacatccagtgtgaggagctgagcgacgctagatgggccgagctcctccctctgctccagcagtgccaagtggtcaggctggacgactgtggcctcacggaagcacggtgcaaggacatcagctctgcacttcgagtcaaccctgcactggcagagctcaacctgcgcagcaacgagctgggcgatgtcggcgtgcattgcgtgctccagggcctgcagaccccctcctgcaagatccagaagctgagcctccagaactgctgcctgacgggggccggctgcggggtcctgtccagcacactacgcaccctgcccaccctgcaggagctgcacctcagcgacaacctcttgggggatgcgggcctgcagctgctctgcgaaggactcctggacccccagtgccgcctggaaaagctgcagctggagtattgcagcctctcggctgccagctgcgagcccctggcctccgtgctcagggccaagccggacttcaaggagctcacggttagcaacaacgacatcaatgaggctggcgttcatgtgctatgccagggcctgaaggactccccctgccagctggaggcgctcaagctggagagctgcggtgtgacatcagacaactgccgggacctgtgcggcattgtggcctccaaggcctcgctgcgggagctggccctgggcagcaacaagctgggtgatgtgggcatggcggagctgtgcccagggctgctccaccccagctccaggctcaggaccctgtggatctgggagtgtggcatcactgccaagggctgcggggatctgtgccgtgtcctcagggccaaggagagcctgaaggagctcagcctggccggcaacgagctgggggatgagggtgcccgactgttgtgtgagaccctgctggaacctggctgccagctggagtcgctgtgggtgaagtcctgcagcttcacagccgcctgctgctcccacttcagctcagtgctggcccagaacaggtttctcctggagctacagataagcaacaacaggctggaggatgcgggcgtgcgggagctgtgccagggcctgggccagcctggctctgtgctgcgggtgctctggttggccgactgcgatgtgagtgacagcagctgcagcagcctcgccgcaaccctgttggccaaccacagcctgcgtgagctggacctcagcaacaactgcctgggggacgcgggcatcctgcagctggtggagagcgtccggcagccgggctgcctcctggagcagctggtcctgtacgacatttactggtctgaggagatggaggaccggctgcaggccctggagaaggacaagccatccctgagggtcatctcc

SEQ ID NO: 17 - DNA, 1137 bp, Artificial Sequence
  Description of Artificial Sequence Synthetic polynucleotide
ggaattcaaggcctggccaaactaattgctgatgtggcccccagtgccatccgggagaatgacatcaagagctactttggccgtaaggtggccattgatgcctctatgagcatttatcagttcctgattgctgttcgccagggtggggatgtgctgcagaatgaggagggtgagaccaccagccacctgatgggcatgttctaccgcaccattcgcatgatggagaacggcatcaagcccgtgtatgtctttgatggcaagccgccacagctcaagtcaggcgagctggccaaacgcagtgagcggcgggctgaggcagagaagcagctgcagcaggctcaggctgctggggccgagcaggaggtggaaaaattcactaagcggctggtgaaggtcactaagcagcacaatgatgagtgcaaacatctgctgagcctcatgggcatcccttatcttgatgcacccagtgaggcagaggccagctgtgctgccctggtgaaggctggcaaagtctatgctgcggctaccgaggacatggactgcctcaccttcggcagccctgtgctaatgcgacacctgactgccagtgaagccaaaaagctgccaatccaggaattccacctgagccggattctgcaggagctgggcctgaaccaggaacagtttgtggatctgtgcatcctgctaggcagtgactactgtgagagtatccggggtattgggcccaagcgggctgtggacctcatccagaagcacaagagcatcgaggagatcgtgcggcgacttgaccccaacaagtaccctgtgccagaaaattggctccacaaggaggctcaccagctcttcttggaacctgaggtgctggacccagagtctgtggagctgaagtggagcgagccaaatgaagaagagctgatcaagttcatgtgtggtgaaaagcagttctctgaggagcgaatccgcagtggggtcaagaggctgagtaagagccgccaaggcagcacccagggccgcctggatgatttcttcaaggtgaccggctcactctcttcagctaagcgcaaggagccagaacccaagggatccactaagaagaaggcaaagactggggcagcagggaagtttaaaaggggaaaa

SEQ ID NO: 18 - DNA, 897 bp, Artificial Sequence
  Description of Artificial Sequence Synthetic polynucleotide
cgcggaatgctcccactcttcgaacctaagggcagagttcttcttgttgacggacaccacttggcatatagaacattccatgcactcaaagggctcacgacctcacggggagaacctgtgcaagctgtgtacggttttgccaagagtttgttgaaggccctcaaggaggatggtgatgctgtaatagttgtatttgatgccaaggctccttctttccgacatgaggcttatggcggctataaggctgggcgggcgcctacaccagaagattttcctcgacaactggcgttgatcaaagagttggttgatttgctcggactcgcccgacttgaggttccgggatacgaagccgacgacgtgttggcatctttggcaaagaaggcggaaaaagaaggatacgaggtacggattcttacagctgacaaggatctgtaccagttgttgtcagatcgcatacacgttttgcatcccgagggttaccttattacacccgcctggctctgggagaaatacggccttcggcccgaccaatgggctgattatcgagccctgacgggtgacgaatcagataacctgcccggcgttaaagggattggtgagaaaacggcccgaaagttgcttgaagaatggggctctttggaggcacttctcaagaacctggaccgcctgaaacctgccatccgcgaaaaaatactcgcacacatggatgatctcaaactcagctgggacttggcgaaagtccgaacagatctgcctctcgaagtggactttgcaaagaggcgggagccagacagggaacgactcagggccttcctggaacgactggaatttggatcattgttgcacgagttcggactcctggaatctggtggtggaggttctggtggtggtggcagc

SEQ ID NO: 19 - DNA, 897 bp, Artificial Sequence
  Description of Artificial Sequence Synthetic polynucleotide
gcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccg

SEQ ID NO: 20 - DNA, 675 bp, Artificial Sequence
  Description of Artificial Sequence Synthetic polynucleotide
acaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcga

SEQ ID NO: 21 - DNA, 966 bp, Artificial Sequence
  Description of Artificial Sequence Synthetic polynucleotide
gttcagatcccccaaaatccacttatccttgtagatggttcatcttatctttatcgcgcatatcacgcgtttcccccgctgactaacagcgcaggcgagccgaccggtgcgatgtatggtgtcctcaacatgctgcgcagtctgatcatgcaatataaaccgacgcatgcagcggtggtctttgacgccaagggaaaaacctttcgtgatgaactgtttgaacattacaaatcacatcgcccgccaatgccggacgatctgcgtgcacaaatcgaacccttgcacgcgatggttaaagcgatgggactgccgctgctggcggtttctggcgtagaagcggacgacgttatcggtactctggcgcgcgaagccgaaaaagccgggcgtccggtgctgatcagcactggcgataaagatatggcgcagctggtgacgccaaatattacgcttatcaataccatgacgaataccatcctcggaccggaagaggtggtgaataagtacggcgtgccgccagaactgatcatcgatttcctggcgctgatgggtgactcctctgataacattcctggcgtaccgggcgtcggtgaaaaaaccgcgcaggcattgctgcaaggtcttggcggactggatacgctgtatgccgagccagaaaaaattgctgggttgagcttccgtggcgcgaaaacaatggcagcgaagctcgagcaaaacaaagaagttgcttatctctcataccagctggcgacgattaaaaccgacgttgaactggagctgacctgtgaacaactggaagtgcagcaaccggcagcggaagagttgttggggctgttcaaaaagtatgagttcaaacgctggactgctgatgtcgaagcgggcaaatggttacaggccaaaggggcaaaaccagccgcgaagccacaggaaaccagtgttgcagacgaagcaccagaagtgacggcaacg

SEQ ID NO: 22 - DNA, 861 bp, Artificial Sequence
  Description of Artificial Sequence Synthetic polynucleotide
aagaagaaattggttctgatcgacggaaactccgttgcgtatagagcgttcttcgcgctccctctcttgcataacgacaagggtatccacacgaacgcggtctacgggttcactatgatgcttaacaaaatcctggctgaggagcaaccaactcacctcctcgtcgcatttgatgctgggaaaacaaccttccggcacgaaacattccaggaatataaaggcggaaggcaacagacgccgccagaactgtcagagcaatttcctctgcttcgagagctccttaaagcttataggataccggcatacgagctcgatcactacgaggcggacgatattatcggaacgcttgctgctcgagcagagcaggagggcttcgaggtcaagattatctccggggaccgagacttgactcaacttgcttcacgccatgtaacagtcgacataacgaaaaaagggattacagatattgaaccctatacaccagagacggtacgcgaaaagtacggcctcaccccagagcagatagttgatctcaaaggtctcatgggcgacaagtcagacaacatcccaggtgtcccagggattggggaaaaaacagctgtcaaacttttgaaacagttcggtacagtggaaaacgttcttgcgtccatagacgaagtaaaaggtgagaagctcaaagagaatcttaggcaacatagagacttggcattgttgtctaaacaactcgcgagtatatgtcgagatgcgcctgtagagctttcccttgacgatattgtgtacgagggacaggaccgggaaaaggtgattgctcttttcaaagaactcggattccagtcttttcttgagaaaatggctgccccc

SEQ ID NO: 23 - DNA, 1764 bp, Artificial Sequence
  Description of Artificial Sequence Synthetic polynucleotide
gcggctgagggtgagaagcctcttgaggagatggagtttgcgatagtcgacgttattactgaggaaatgctcgctgataaagccgcgctcgttgttgaggtaatggaagagaactatcatgacgcccccatcgtcggtatagcgctggtaaacgaacatgggcgatttttcatgcggcccgaaacagcgttggcagacagtcaatttcttgcctggcttgcagacgagacgaagaaaaaaagcatgtttgacgcgaaacgcgcggtagtggcactcaaatggaagggcatcgagctcaggggtgtagccttcgatctcctgctcgctgcgtaccttcttaatcccgcgcaggatgcaggcgacatagccgctgtcgcaaagatgaagcaatatgaggcggtccgatccgatgaagccgtttacggcaagggcgtgaaacggagtctccctgatgagcaaacacttgcggaacatcttgtgcgaaaagccgcagcgatatgggctctggaacagccatttatggatgacttgcgaaacaacgagcaagatcagctgttgacgaagttggaacaaccgcttgcggcgatactggcggagatggaattcacgggggtgaacgttgatacgaaaaggcttgagcagatgggatcagaactcgctgaacaacttagagccatcgaacaaagaatatacgaacttgcggggcaggaattcaatataaatagcccaaaacaacttggggtcatactctttgagaagcttcaactccccgtattgaaaaagacgaagacggggtatagtacaagtgcggatgtcctggaaaagttggcgccgcatcacgaaattgtagaaaatatactgcattacaggcaacttgggaaactccaatcaacgtacatagaaggactccttaaagttgtccgacctgatacaggcaaggtccacacgatgtttaatcaagcacttacgcaaaccggtcgcctgagctctgcggagccaaatctccagaatataccgattcggctggaagaaggtcgcaaaattcggcaggcgttcgtacctagcgaacctgattggcttatattcgcggcggattactctcagatagagcttagggtattggctcacattgccgatgacgacaacttgattgaagcgttccagcgcgatttggacatacatactaagacagcaatggatatcttccacgtgtctgaggaggaggtaactgctaacatgcggcggcaggcaaaggccgtaaactttggtattgtttatggaataagcgactacgggctcgcccagaaccttaacatcacacgcaaagaagccgccgagtttattgagagatatttcgcaagtttccccggagtaaaacaatacatggagaatatcgtacaagaggctaagcagaagggctatgtcaccacattgctccacagaagacggtatttgccagacattactagtcgaaactttaacgtgaggtcattcgcagagcggacggcgatgaatacacccattcaaggaagtgcagctgacattatcaaaaaggccatgattgacctcgcagctaggttgaaagaagaacagctccaggcccgcctgctgctccaggtgcatgatgagctcatactcgaagccccgaaggaggaaatagaacggctgtgcgagttggtcccagaagtaatggagcaagctgtcacgctccgagttccccttaaggtggactaccattatggtccaacgtggtatgatgctaag

SEQ ID NO: 24 - DNA, 2625 bp, Artificial Sequence
  Description of Artificial Sequence Synthetic polynucleotide
aagaagaaattggttctgatcgacggaaactccgttgcgtatagagcgttcttcgcgctccctctcttgcataacgacaagggtatccacacgaacgcggtctacgggttcactatgatgcttaacaaaatcctggctgaggagcaaccaactcacctcctcgtcgcatttgatgctgggaaaacaaccttccggcacgaaacattccaggaatataaaggcggaaggcaacagacgccgccagaactgtcagagcaatttcctctgcttcgagagctccttaaagcttataggataccggcatacgagctcgatcactacgaggcggacgatattatcggaacgcttgctgctcgagcagagcaggagggcttcgaggtcaagattatctccggggaccgagacttgactcaacttgcttcacgccatgtaacagtcgacataacgaaaaaagggattacagatattgaaccctatacaccagagacggtacgcgaaaagtacggcctcaccccagagcagatagttgatctcaaaggtctcatgggcgacaagtcagacaacatcccaggtgtcccagggattggggaaaaaacagctgtcaaacttttgaaacagttcggtacagtggaaaacgttcttgcgtccatagacgaagtaaaaggtgagaagctcaaagagaatcttaggcaacatagagacttggcattgttgtctaaacaactcgcgagtatatgtcgagatgcgcctgtagagctttcccttgacgatattgtgtacgagggacaggaccgggaaaaggtgattgctcttttcaaagaactcggattccagtcttttcttgagaaaatggctgcccccgcggctgagggtgagaagcctcttgaggagatggagtttgcgatagtcgacgttattactgaggaaatgctcgctgataaagccgcgctcgttgttgaggtaatggaagagaactatcatgacgcccccatcgtcggtatagcgctggtaaacgaacatgggcgatttttcatgcggcccgaaacagcgttggcagacagtcaatttcttgcctggcttgcagacgagacgaagaaaaaaagcatgtttgacgcgaaacgcgcggtagtggcactcaaatggaagggcatcgagctcaggggtgtagccttcgatctcctgctcgctgcgtaccttcttaatcccgcgcaggatgcaggcgacatagccgctgtcgcaaagatgaagcaatatgaggcggtccgatccgatgaagccgtttacggcaagggcgtgaaacggagtctccctgatgagcaaacacttgcggaacatcttgtgcgaaaagccgcagcgatatgggctctggaacagccatttatggatgacttgcgaaacaacgagcaagatcagctgttgacgaagttggaacaaccgcttgcggcgatactggcggagatggaattcacgggggtgaacgttgatacgaaaaggcttgagcagatgggatcagaactcgctgaacaacttagagccatcgaacaaagaatatacgaacttgcggggcaggaattcaatataaatagcccaaaacaacttggggtcatactctttgagaagcttcaactccccgtattgaaaaagacgaagacggggtatagtacaagtgcggatgtcctggaaaagttggcgccgcatcacgaaattgtagaaaatatactgcattacaggcaacttgggaaactccaatcaacgtacatagaaggactccttaaagttgtccgacctgatacaggcaaggtccacacgatgtttaatcaagcacttacgcaaaccggtcgcctgagctctgcggagccaaatctccagaatataccgattcggctggaagaaggtcgcaaaattcggcaggcgttcgtacctagcgaacctgattggcttatattcgcggcggattactctcagatagagcttagggtattggctcacattgccgatgacgacaacttgattgaagcgttccagcgcgatttggacatacatactaagacagcaatggatatcttccacgtgtctgaggaggaggtaactgctaacatgcggcggcaggcaaaggccgtaaactttggtattgtttatggaataagcgactacgggctcgcccagaaccttaacatcacacgcaaagaagccgccgagtttattgagagatatttcgcaagtttccccggagtaaaacaatacatggagaatatcgtacaagaggctaagcagaagggctatgtcaccacattgctccacagaagacggtatttgccagacattactagtcgaaactttaacgtgaggtcattcgcagagcggacggcgatgaatacacccattcaaggaagtgcagctgacattatcaaaaaggccatgattgacctcgcagctaggttgaaagaagaacagctccaggcccgcctgctgctccaggtgcatgatgagctcatactcgaagccccgaaggaggaaatagaacggctgtgcgagttggtcccagaagtaatggagcaagctgtcacgctccgagttccccttaaggtggactaccattatggtccaacgtggtatgatgctaag

SEQ ID NO: 25 - DNA, 336 bp, Artificial Sequence
  Description of Artificial Sequence Synthetic polynucleotide
gcgatgcagatgcagttggaagcgaatgcagatactagtgtcgaggaagagtcatttggcccgcaacccatctcgcgtttagagcaatgtggcatcaatgcaaacgatgtgaaaaaattagaggaagctggattccacacggtcgaagcggtcgcatacgcaccgaaaaaagagctgatcaacatcaaaggcatcagcgaggcgaaagccgataagattcttgcagaggcggcgaaattagttcccatgggatttacgacggcgactgagttccatcaacgtcgttccgagatcattcaaatcacgaccggaagcaaggagttggataaactgctt

SEQ ID NO: 26 - DNA, 246 bp, Artificial Sequence
  Description of Artificial Sequence Synthetic polynucleotide
ggcgtgctcagggtcggactgtgccctggccttaccgaggagatgatccagcttctcaggagccacaggatcaagacagtggtggacctggtttctgcagacctggaagaggtagctcagaaatgtggcttgtcttacaaggccctggttgccctgaggcgggtgctgctggctcagttctcggctttccccgtgaatggcgctgatctctacgaggaactgaagacctccactgccatcctgtcc

SEQ ID NO: 27 - DNA, 447 bp, Artificial Sequence
  Description of Artificial Sequence Synthetic polynucleotide
ggcagtgatggtgatagtgctaatgacactgaaccagactttgcacctggtgaagattctgaggatgattctgatttttgtgagagtgaggataatgacgaagacttctctatgagaaaaagtaaagttaaagaaattaaaaagaaagaagtgaaggtaaaatccccagtagaaaagaaagagaagaaatctaaatccaaatgtaatgctttggtgacttcggtggactctgctccagctgccgtcaaatcagaatctcagtccttgccaaaaaaggtttctctgtcttcagataccactaggaaaccattagaaatacgcagtccttcagctgaaagcaagaaacctaaatgggtcccaccagcggcatctggaggtagcagaagtagcagcagcccactggtggtagtgtctgtgaagtctcccaatcagagtctccgccttggc

SEQ ID NO: 28 - DNA, 726 bp, Artificial Sequence
  Description of Artificial Sequence Synthetic polynucleotide
gacgaagaggaactcatccagttgataatagaaaaaactggtaagtcccgcgaagaaatagagaagatggttgaggagaaaataaaggcgttcaacaatctcatctcacgaagaggagctttgctcctcgtggcaaagaaacttggagtattatacaagaacacgccgaaggaaaaaaaaattggcgagcttgaatcctgggagtatgttaaggttaaaggcaagatactgaagagctttgggcttatttcttacagcaaaggcaagttccagcccattattctgggagacgaaactggcacaattaaggcgattatatggaacaccgacaaagaattgccagagaacacagttatagaagctataggtaagaccaagatcaacaagaaaactgggaatcttgaacttcatatagactcctataaaatcctcgaatccgatcttgagataaaacctcaaaagcaagaatttgttgggatctgtattgtgaagtaccccaagaaacaaacacagaaagggacaatcgtttctaaagcgatattgaccagtctcgatagggaacttcccgtggtgtacttcaatgacttcgattgggaaattggccatatctataaggtgtatggaaaactgaaaaagaatataaaaacgggaaaaatcgagtttttcgcggataaggtggaagaagccacgcttaaggatctcaaagcgtttaagggcgaagctgac

SEQ ID NO: 29 - DNA, 2457 bp, Artificial Sequence
  Description of Artificial Sequence Synthetic polynucleotide
agtagtcgtggtttcaggtctaataactttattcaagcacaattgaagcatccttccatactttcaaaagaagacctagatttgctctctgattcggatgattgggaagaacctgattgcatacagttagaaactgagaagcaagaaaagaaaattatcactgacatacataaagaagacccggtggacaaaaagcctatgagggataaaaatgtcatgaattttatcaataaagacagtcctttatcctggaacgatatgtttaaacccagtataatacaaccaccgcagttaatttctgaaaactcatttgaccagagcagtcaaaaaaaatcgagatcgacaggattcaagaatccattaagaccagcgttgaaaaaggaaagttcttttgatgaacttcaaaataattctatatctcaagagagaagtttggaaatgataaatgaaaacgaaaagaagaaaatgcaatttggagaaaagattgctgttttgacgcaaagacctagcttcactgaattgcagaatgaccaagatgacagtaacttgaatccccataatggtgtgaaagtcaagataccgatttgcttaagcaaagaacaagaaagtatcatcaagttggcagaaaatggccacaacattttttatacagggagtgccggtaccggtaaatccattcttttacgtgaaatgataaaagttttaaaaggcatatatggtagggagaatgttgcagtcactgcttccacgggtttagctgcttgtaatatcggtggtataaccatacactcgttcgctggtataggattaggaaaaggtgatgcggataaactctataaaaaagttcgtaggtctcgaaagcacctaaggcgctgggaaaatattggtgctttggttgtcgatgaaatatcaatgttagacgcagaactgcttgataaactcgatttcatagctagaaaaatacggaaaaatcatcaacccttcggtggaattcaactcatcttctgtggcgattttttccagttaccgccagtatcaaaagatcctaatagaccaactaagtttgctttcgaatccaaggcttggaaagaaggtgtaaagatgacgattatgctacaaaaggtttttagacagcgaggcgatgttaagttcattgacatgttgaatcggatgagactaggcaatattgatgatgaaacagaaagagagttcaagaagctttctagaccattgccagacgatgaaattattcccgcggaactttatagtaccagaatggaagtagaaagggccaataattcaaggctaagtaaattgccaggccaggtgcatatttttaatgcaatcgatggcggtgctttggaagacgaagagttaaaggaaaggctgttacaaaattttttagctccaaaggaattacatttgaaagttggcgctcaggttatgatggtaaaaaatctagacgcaacattagttaatggatcccttggtaaagtcatcgaattcatggatccagaaacatatttttgctatgaggcgctaacaaacgatccatctatgcctccagaaaaactcgagacttgggcagaaaacccttcaaaactaaaagctgcaatggagagggagcaaagtgatggggaagaaagtgcggtagctagtcgcaaatcttcagtgaaggagggatttgctaagagtgatataggtgagccggtctctcccctagattcctcagtttttgacttcatgaagagagtcaagacagatgacgaagttgtgctggaaaatataaaacgcaaggaacaactgatgcagaccatacatcaaaactctgcaggaaaacgaaggttacctctcgtgagattcaaagcttctgatatgagtacgaggatggtgcttgtcgagccggaggattgggcgatagaagacgaaaatgaaaagccactggtatcaagggttcaattaccgctaatgcttgcctggtcactatccattcacaaatctcagggtcagacacttccaaaagttaaagtggatttacgtagagtattcgaaaagggtcaggcgtatgttgccctttctagagctgtttcaagagaaggactacaggtgttaaattttgacagaactaggatcaaagcacatcaaaaggtaattgatttttatcttactttatcttcagccgaaagtgcctataagcaacttgaggcagatgagcaagtgaaaaaaaggaagttagactacgcaccaggccctaaatataaggctaaatccaagtcaaagtcaaattctccagcacccatatcagcgaccacacaatctaataatggtatcgcagcgatgttgcaaagacacagtaggaagagatttcagttgaaaaaagagtctaatagtaatcaagttcattcattggtttccgacgaacctcgtggtcaggataccgaagaccacatcttagaa

SEQ ID NO: 30 - DNA, 2322 bp, Artificial Sequence
  Description of Artificial Sequence Synthetic polynucleotide
attcttgacacggattacatcacggaagacggcaagccggttatccgtattttcaagaaagaaaacggcgaattcaagattgaatacgatcggacatttgaaccgtacctgtacgctctcctcaaggatgatagcgcaatcgaagaagtgaaaaaaatcaccgcagagcggcatggcacagtggtaacagttaagcgggtcgagaaagtgcagaagaagttcttaggccggccagtcgaagtatggaaattatacttcacacatccacaggacgttccggcgatcatggataagattcgggagcatccggcggtaatcgatatctatgaatacgatattccgttcgctattcgctaccttattgacaaaggtttagttccaatggagggtgatgaggaacttaaactgttagcattcgatatcgaaacactttatcacgaaggtgaagagtttgccgaaggtccgattttaatgatctcatacgccgatgaagaaggcgcacgcgtaattacgtggaaaaatgtggacctcccatacgtagacgtagtgagcactgagcgcgagatgattaaacgtttccttcgggtagtaaaagaaaaagacccagacgtgctgattacgtataacggcgacaactttgattttgcctatctcaagaagcgttgcgaaaagttaggcattaatttcgccctgggtcgggacggttcagagccgaaaattcagcggatgggcgaccgctttgctgtggaggtaaaaggtcgcatccatttcgatttatatccggttatccggcgcaccatcaacttgccgacttacacacttgaagcagtttacgaagcggtgttcggccaaccaaaagaaaaggtttatgccgaggagattaccaccgcatgggaaactggcgaaaacttggagcgggtggctcggtattccatggaagatgccaaggtgacctacgaactgggcaaagagtttttaccgatggaagcacaattaagccgccttattggtcagtccctctgggatgtgtcgcgttcttcaacgggcaatttagtcgaatggtttcttcttcggaaagcatacgagcgtaacgagcttgctccaaataagccagacgaaaaagaattggctcggcgccatcagtcacatgagggcggctacattaaggagccagaacggggcttgtgggagaacatcgtctaccttgattttcggtctctttatccgtctattatcatcacacataacgtctcgccagataccctgaaccgtgaaggctgtaaagaatatgatgtggcaccacaggtcggccatcgtttttgtaaagacttcccgggcttcattccatctcttctgggtgatttgttagaagagcgtcaaaagatcaagaaacgtatgaaagcgacaattgacccaattgaacgcaaattacttgattaccgtcagcgtgcaatcaagatcctcgcgaactctctgtacggttattacggctacgcacgcgcccggtggtattgcaaagaatgtgcagaatcagtcattgcttggggtcgggagtacctgaccatgacgattaaggaaattgaggagaaatacggtttcaaggtcatctatagtgacacggatggtttctttgcaacgattccaggtgcggacgcagaaactgtaaagaaaaaggcaatggagttcttgaagtatattaatgcgaagttgccaggcgccctggaattagagtacgaaggtttttataagcgtggcctgttcgtgacaaagaagaaatacgcggtaattgacgaggaaggcaagatcacaactcgtggcttggaaattgttcgtcgcgattggagcgagatcgcaaaggagacccaagctcgtgtgttggaggccctcctgaaggatggtgacgtcgaaaaagcagtacgcatcgttaaggaggttacagagaagcttagcaagtatgaggtcccaccagagaaacttgttattcataaacaaatcactcgcgaccttaaagactataaggccactggtccacacgtcgccgtagcaaagcggcttgcggctcggggcgtcaagattcggccaggcacggttattagttacatcgtcctcaaaggctcaggccggattgttgatcgcgcgattccatttgatgaatttgatccgacgaagcataaatatgatgcggaatattacattgaaaaacaggttctgccggcggtggagcgcatcttacgtgcgttcggctatcgcaaggaggatttgcggtaccagaaaactcgtcaagtcggtttgagtgcctggctgaagccgaaaggtacctga

SEQ ID NO: 31 - DNA, 1728 bp, Artificial Sequence
  Description of Artificial Sequence Synthetic polynucleotide
aacacaccaaaacccattctcaaaccgcaatctaaggccttggtagagcccgtactttgtgattctatcgacgagatcccggccaagtacaacgagcccgtgtattttgacttggaaacggatgaagatcgaccagtactcgcatccatatatcaacctcattttgaaaggaaagtctattgtctcaacttgctgagggaaaagttggcccgctttaaggagtggcttctcaagttttccgagatccgagggtggggacttgacttcgacctccgagtgttgggctacacatacgaacagctgaggaataagaagattgtagacgtccaactcgcgataaaggtacagcactatgagcgattcaagcaaggagggacgaagggagaaggctttagattggacgacgttgcccgagatctgttgggtatcgagtatccaatgaacaaaacgaaaataagaacgacctttaagtataacatgtactctagcttctcttacgagcaattgctgtacgcaagcctcgacgcatacattcctcacctgctgtatgagaggcttagcagtgacacgctcaattctttggtataccaaatagatcaagaggtgcagaaagttgtcatagaaacatctcagcatggcatgcccgtaaaactgaaagcactggaggaagaaatacatagactcacacagcttaggtcagaaatgcaaaaacagattcccttcaactacaattctcctaagcagacagcgaagtttttcggcgttaactcttcttcaaaggacgtcctcatggatcttgccctcaggggcaacgaagttgcgaaaaaagtgctggaggcaagacaaatcgagaagtccctggcattcgcgaaggacctctacgatatagccaagaaaaatggcggccgaatttatggaaatttcttcacgacgacagcccccagcggaaggatgagctgctcagatatcaatttgcagcagatcccgcgacggcttaggccgttcataggttttgaaacggaggataagaagcttatcaccgctgacttcccacagatcgaacttcggctggctggggttatgtggaacgaacctgagttcctgaaagcctttcgggacggaatagatctccataaattgacggccagcattctcttcgataaaaaaataaatgaggtgagcaaagaagagcgccaaattggtaaatcagcgaattttggcttgatttacggaatttctccgaaagggttcgcggagtattgcatctccaatggaatcaatataacagaggagatggcaatcgaaatcgtcaagaaatggaagaagttctatcgcaagatagccgaacagcaccaactcgcctacgaacggttcaaatacgctgagttcgttgataatgaaacctggttgaacaggccctatcgcgcttggaaaccccaggacctcctcaactatcaaatccaaggcagtggagctgaactcttcaagaaagcaatcgtgttgttgaaagaagcaaagccagatctcaaaattgtgaacctcgtgcatgatgaaatagtggtcgagacctccaccgaggaagcagaagatattgcactccttgttaaacaaaagatggaagaggcttgggactactgcctggagaaggccaaggaatttggtaataacgtcgctgatattaagcttgaggttgagaaaccaaacatatccagcgtctgggaaaaagaa

SEQ ID NO: 32 - DNA, 2094 bp, Artificial Sequence
  Description of Artificial Sequence Synthetic polynucleotide
accctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaacactagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagtttctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtc

SEQ ID NO: 33 - DNA, 2625 bp, Artificial Sequence
  Description of Artificial Sequence Synthetic polynucleotide
cgcggaatgctcccactcttcgaacctaagggcagagttcttcttgttgacggacaccacttggcatatagaacattccatgcactcaaagggctcacgacctcacggggagaacctgtgcaagctgtgtacggttttgccaagagtttgttgaaggccctcaaggaggatggtgatgctgtaatagttgtatttgatgccaaggctccttctttccgacatgaggcttatggcggctataaggctgggcgggcgcctacaccagaagattttcctcgacaactggcgttgatcaaagagttggttgatttgctcggactcgcccgacttgaggttccgggatacgaagccgacgacgtgttggcatctttggcaaagaaggcggaaaaagaaggatacgaggtacggattcttacagctgacaaggatctgtaccagttgttgtcagatcgcatacacgttttgcatcccgagggttaccttattacacccgcctggctctgggagaaatacggccttcggcccgaccaatgggctgattatcgagccctgacgggtgacgaatcagataacctgcccggcgttaaagggattggtgagaaaacggcccgaaagttgcttgaagaatggggctctttggaggcacttctcaagaacctggaccgcctgaaacctgccatccgcgaaaaaatactcgcacacatggatgatctcaaactcagctgggacttggcgaaagtccgaacagatctgcctctcgaagtggactttgcaaagaggcgggagccagacagggaacgactcagggccttcctggaacgactggaatttggatcattgttgcacgagttcggactcctggaatctggtggtggaggttctggtggtggtggcagcaacacaccaaaacccattctcaaaccgcaatctaaggccttggtagagcccgtactttgtgattctatcgacgagatcccggccaagtacaacgagcccgtgtattttgacttggaaacggatgaagatcgaccagtactcgcatccatatatcaacctcattttgaaaggaaagtctattgtctcaacttgctgagggaaaagttggcccgctttaaggagtggcttctcaagttttccgagatccgagggtggggacttgacttcgacctccgagtgttgggctacacatacgaacagctgaggaataagaagattgtagacgtccaactcgcgataaaggtacagcactatgagcgattcaagcaaggagggacgaagggagaaggctttagattggacgacgttgcccgagatctgttgggtatcgagtatccaatgaacaaaacgaaaataagaacgacctttaagtataacatgtactctagcttctcttacgagcaattgctgtacgcaagcctcgacgcatacattcctcacctgctgtatgagaggcttagcagtgacacgctcaattctttggtataccaaatagatcaagaggtgcagaaagttgtcatagaaacatctcagcatggcatgcccgtaaaactgaaagcactggaggaagaaatacatagactcacacagcttaggtcagaaatgcaaaaacagattcccttcaactacaattctcctaagcagacagcgaagtttttcggcgttaactcttcttcaaaggacgtcctcatggatcttgccctcaggggcaacgaagttgcgaaaaaagtgctggaggcaagacaaatcgagaagtccctggcattcgcgaaggacctctacgatatagccaagaaaaatggcggccgaatttatggaaatttcttcacgacgacagcccccagcggaaggatgagctgctcagatatcaatttgcagcagatcccgcgacggcttaggccgttcataggttttgaaacggaggataagaagcttatcaccgctgacttcccacagatcgaacttcggctggctggggttatgtggaacgaacctgagttcctgaaagcctttcgggacggaatagatctccataaattgacggccagcattctcttcgataaaaaaataaatgaggtgagcaaagaagagcgccaaattggtaaatcagcgaattttggcttgatttacggaatttctccgaaagggttcgcggagtattgcatctccaatggaatcaatataacagaggagatggcaatcgaaatcgtcaagaaatggaagaagttctatcgcaagatagccgaacagcaccaactcgcctacgaacggttcaaatacgctgagttcgttgataatgaaacctggttgaacaggccctatcgcgcttggaaaccccaggacctcctcaactatcaaatccaaggcagtggagctgaactcttcaagaaagcaatcgtgttgttgaaagaagcaaagccagatctcaaaattgtgaacctcgtgcatgatgaaatagtggtcgagacctccaccgaggaagcagaagatattgcactccttgttaaacaaaagatggaagaggcttgggactactgcctggagaaggccaaggaatttggtaataacgtcgctgatattaagcttgaggttgagaaaccaaacatatccagcgtctgggaaaaagaa

SEQ ID NO: 34 - DNA, 1815 bp, Artificial Sequence
  Description of Artificial Sequence Synthetic polynucleotide
caagtcgggcatagaaaaattaggccacataatatagcaactggtgattatcctcctcgccctcaaaaacaatatcctattaatcctaaggcaaagcctagtatacaaattgtaatagatgacttattgaaacaaggggtgttaacgcctcaaaatagtacaatgaatacaccagtgtatcctgttcctaaaccagatggaaggtggagaatggtattagattatagagaagtaaataaaactattccattaacagctgcccaaaaccaacactctgctggtattttagctactattgttagacaaaaatataaaactaccttagatttagctaatggattttgggctcatcctattacaccagaatcttattggttaacagcatttacctggcaaggtaaacagtattgttggacacgtcttcctcaaggatttttaaatagtccagcattgtttacagctgatgtagtagatttactaaaagaaatccctaacgtacaagtgtatgttgatgatatatatttaagccatgatgatcctaaagagcatgttcaacaattagaaaaagtgtttcaaattttactacaggcaggatatgtagtatctttgaaaaaatcagaaattggtcaaaaaactgtagaatttttaggatttaatattactaaagaaggtcgtggcctaacagacacttttaaaacaaaactgttaaatattactcctccaaaagacttaaagcaattacaaagcatattaggattgttaaattttgctagaaattttatacctaattttgctgaactggtacaaccattatacaatttaatagcctcagcaaaaggcaaatatattgagtggtctgaagaaaatactaaacaattaaatatggtaatagaagcattaaacactgcctctaatttagaagaaaggttaccagaacagagactggtaattaaagtcaatacttctccatcagcaggatatgtaagatattataatgagactggtaaaaagcctattatgtacctaaattatgtgttttccaaagcagaattaaaattttctatgttagaaaaactattaactacaatgcacaaagccttaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaagtctattgctgagtgtttatctatgaaaccagacattactattcaacatgaaaaagggcatcagcctacaaataccagtattcatactgaaggcaatgccctagcagataagcttgccacccaaggaagttat

SEQ ID NO: 35 - DNA, 984 bp, Artificial Sequence
  Description of Artificial Sequence Synthetic polynucleotide
ggaaaaaggcacaggaaccttatagatcagattacgacgtgggaaaatctcttggacgcgtaccgaaaaactagccacggtaaaagacgaacatggggttacctggagttcaaagagtacgacttggcaaatttgttggcgctccaagcggaactgaaggctggaaactacgaaagaggcccttaccgcgaatttctggtatatgaaccgaaaccacggcttatatctgctcttgaattcaaggatagactcgtgcagcatgcactttgtaatatagttgccccgatatttgaagcggggcttctgccatatacatacgcatgtcggccggacaaggggactcatgcgggcgtttgtcatgtccaggcagagcttcgacgaacacgagcgactcattttctcaaatccgatttcagtaaattcttccccagtattgatcgagcggctctttatgccatgatcgacaaaaagattcactgcgccgccactcggagactcttgagggtggtcctgccggatgaaggagtaggcataccgattggtagcctgacgagtcaactttttgccaacgtatacggcggggcagtggatcgccttcttcacgatgaacttaaacaacgccattgggctaggtatatggatgacatcgtggttttgggggatgatcccgaagaattgcgagcggtgttctaccggcttcgagacttcgccagcgagagacttggccttaaaataagtcattggcaggttgcccccgtgagcaggggcataaatttcctgggctatcggatttggccgacgcataagctccttcgaaagtctagtgtcaagagggccaaaagaaaggtagcaaactttattaaacacggcgaggacgaaagtcttcagcgcttcttggcgagctggagcgggcatgcccaatgggctgacacgcacaatttgttcacttggatggaggagcagtacggaatcgcgtgtcattag

SEQ ID NO: 36 - DNA, 984 bp, Artificial Sequence
  Description of Artificial Sequence Synthetic polynucleotide
aaacgcaagggcaacttgtatcacaaaattacagaatggaacaacctgatagccgcattttacaacgctagtagaggcaagaggcttaagccggatgtcctgctgtacgaaaagaacctttacacaaatttgaagaccctgcaaaattatctgataaaccagaccgttctcctcggtagctaccggtttttcaaaatttacgatccgaaggaacgcatcatatgtgcggccccgttcaatgaacgagtacttcaccacgcgataataaatataacagagagcgtctttgaaaagttccaaatttacgattcctacgcttgtagaaaaaacaaggggacgcaagccgcattgttgagggctctctacttttcccggcggttcaaatacttcctgaaattggatatgaaaaagtactttgattctatacctcattccaagctctccctgcttctgacctgcaaattcaaggataaggcgttgctgcatttgtttaacaaacttatcgcatcttacagcgtaactgaagggtggggcgtgcctataggcaatttgacgagtcagtacttcgccaatttttatctgtcttttttcgatcactatgctaaggaaaaaatgaatgtccgggggtatatccggtacatggatgatgtgctgttgttctccgataacctcaaagatattaaactgatccaaaagaaagctaaaaattttctcagctgcgaactggatctcaccttgaaggaggagataattggtatggtgaagaatggcatcccgtttctcggattcctcgtgaaaccacaagggatctacttgagccaaaaaaagaagaaaaggctgaagaagaaaattaaagattacgttcacaagtttaagattgcttattggacggaggaggagtttgctttgcacattacgccagttttcgcccacattgcgatatcccgatgtcgcgcatactgtaacaaatacctcttgacatag

SEQ ID NO: 37 - DNA, 1293 bp, Artificial Sequence
  Description of Artificial Sequence Synthetic polynucleotide
tggagggaagacaatattatcgaagaaatagtcgaagatagcaacatcgaagatgcgataaagaccgtactgaggaagcgcaggcgaaaacggtcatttgcgggtcgcaggattctggcggatgtcccaaaagcggtggagcggattaggaaaaggatacgaagtgggaggtttaagctcggtggctacagagagatgacggtagacgatgggcccaaggtgcgcatagttcaggccgtgagcctcgaagaccgcatcgttcttaatgccgtcatgaatgtagtagataggcacttgaaggtcagattcatacgcacgaccagtgcctccatcaagaaccgaggcactcacgatctcctccaatatatcgtgaaggatattaaggacgatcctgaggggacgcttttcggctatcaatttgacataacgaaattttacgagtcagttgaccaggatgtgctgctcgacgccgtaaaacgcatgtttaaagacaaaatcttgataggtatcctcgaagaatgcatcagaatgatgcctaagggggtatcaatcggattgagatcctcccagggcctctgcaaccttctcctctctatatatttggatcatcggcttaaagatcaagaggctgtcgcacattattacaggtattgcgatgacggtctcgtcctcagcggctctaaaaaatatttgtggaaagtccgggatatcatccacgaacaaactaggaaagcccggttggaaataaaatctaatgatactgtgttccctatcacagaaggaatcgatttccttggttacgtcaccaggcccgatcacgtgaggctcagaaagcggaataagcaaaaattcgcccgcaaaatgcacaagattaaatcaaagaagcgccgccaagagctgacagcttctttttacggtttgactaagcatgcggactgtaaaaacttgttctataagctgacaggcaagaaaatgaagaagcttaaagatttgggatacaagtacaagcccaaggatggaagaaagcggtttacagggacccgaatcaaatctcccgaactgatgaacaaggatgtaatcgttttggattatgaaaaagatgtccctaccaagaatggtaatcgaacagttatcaaactggagctcgatggcaaggaacggaagtatttcacgtctctcgaagaaactctctttatatgtgaatctgctgcgaaggatggcgaactgccatttgaggcccattgtgagggggaagtatccgagaaaggtctcattatcattcacttcacatag

SEQ ID NO: 38 - DNA, 1146 bp, Artificial Sequence
  Description of Artificial Sequence Synthetic polynucleotide
aactcagatgaacgcagggccgcaagacgcgcgagaagagaagctgagcgggcacgacgcaaagcagagcgcaacgcaggttgtgacctcgaagcagtggccgatcttaatgctctctacaaagcggcgaaacaggcggcccgaggagtggcatggaaggcatcagttcaaagatatcaggctgatgttttgcgaaacgtaatgaaggctcggagagacttgcttgaggggagggatgtctgtcgaggattcataaggttcgacctctgggagcgcgggaagcttaggcacatcagtgcggtacgatttagtgaacgggtcatacaaaaaagtctcacacagaatgcactggttccagctatagcaccgacactcacgtatgacaattcagcaaacttgaaagggaaaggaactgactttgccattgcacggatgaaaaagcagttggctagattttataggaaacacggcgccgatgggtatatcctgctggtggatttttctgattacttcgcaagaatctctcatggccctgctaaggcaattgttgctggggcccttgaggataggcggctcgtagcgttggaacaccggttcattgacgcacagggagacattgggctcggtctcggcagtgaacccaaccagattcttgctgtagcatttccatcttatatagatcacttcgcagctgaaatgtgcggactggaggccaccggccggtatatggatgactcatattatatacacgagtctaaagcatatctcgaagttgtattgatgctgatagagcagaagtgcgatcaatgtggcatttcaatcaatagaaagaagacaagaatcgtaaaactgtcccgagggttcacattcctgaaaaagaaaatttcctttggtgagaatgggagaatcgtagtccgcccatcacgagagagtataacacgcgagcgacggaaactgaagaaacaaagaaaacttgtcgacctgggtatgatgactccagaacaggtggaacgcagttatcagagttggagaggcggcatgaaaaagttggatgcgcatagaacggtactgtccatggacgcattgtataaagatctcttctcaaaccctgaaaatgcgtcaaggggtggagtgtcattgaaataa

SEQ ID NO: 39 - DNA, 288 bp, Artificial Sequence
  Description of Artificial Sequence Synthetic polynucleotide
agcactgacgttgagcctagccctgcacggccggcattgcgggcacccgcctcagctactagcgggagcaggaagagagccaggccccctgcagcacctggcagggaccaggccaggccacccgctcgcagacgacttcgcctgtccgtcgatgaggtctcatccccttccacccccgaagcacctgacatacccgcctgtcctagtcccggtcagaagattaagaaatccacccccgccgccggccaaccaccccacctgaccagcgcccaggatcaggacaccatt

SEQ ID NO: 40 - DNA, 591 bp, Artificial Sequence
  Description of Artificial Sequence Synthetic polynucleotide
agcactgacgttgagcctagccctgcacggccggcattgcgggcacccgcctcagctactagcgggagcaggaagagagccaggccccctgcagcacctggcagggaccaggccaggccacccgctcgcagacgacttcgcctgtccgtcgatgaggtctcatccccttccacccccgaagcacctgacatacccgcctgtcctagtcccggtcagaagattaagaaatccacccccgccgccggccaaccaccccacctgaccagcgcccaggatcaggacaccattggaagcggctctggcagtaccgacgtggaaccatctccagctcgacccgccctcagggccccagcatctgcgacaagtggcagtcgcaagagagcacggcctcctgccgcacccggtcgggaccaggcacgcccccccgcaagacgccgacttagactgtcagttgatgaagtgtccagcccctctacacctgaggcacctgatattcctgcttgcccaagtcctggacagaaaatcaagaagagcacgcccgccgcaggtcagcctccacacctcacgtctgcgcaggaccaagacaccatt

SEQ ID NO: 41 - DNA, 756 bp, Artificial Sequence
  Description of Artificial Sequence Synthetic polynucleotide
gacatagttatgactcaaaccccgctttccctcccagtctcactgggggatcaagcgtccatctcatgccgctcttcacagagtattgtgcattctaacggtaacacatacctggaatggtatttgcaaaagccaggtcaaagcccaaagcttctcatctataaggtttcaaataggttttctggcgtcccagatcgattctccgggagtgggtctggtactgattttactcttaagatatcaagagtcgaggccgaggacttgggggtctattactgtttccaagggagccacgttccatatacttttgggggtgggacaaaactggaaataaaacgagggggcggagggtccggaggaggggggagtggcggaggagggtcaggtggcggaggatcccaggtgcagttgcaacagtcaggtccagaattggttaaacctggcgcgtctgtaaaaatgtcctgtaaagcgtccggatacacgtttacgagttacgttatgcactgggtgaaacagaaaccggggcagggcctggaatggatcgggtttatcaacttatacaacgatggaacaaagtacaatgaaaagtttaaaggcaaagccacgttgacttcagataaaagctcatcaactgcatatatggagctgtcatctcttacttccaaggatagcgcggtttattactgtgctcgggattattatggaagcagatggtttgactattggggacaagggacgacattgactgtatctagc

SEQ ID NO: 42 - DNA, 231 bp, Artificial Sequence
  Description of Artificial Sequence Synthetic polynucleotide
ggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaaggtggtggtagcggtggtggtactagtcccaagaagaagcgcaaggtg

SEQ ID NO: 43 - DNA, 2496 bp, Artificial Sequence
  Description of Artificial Sequence Synthetic polynucleotide
tctaaccaaatatactcagcgagatattcgggggttgatgtttatgaattcattcattctacaggatctatcatgaaaaggaaaaaggatgattgggtcaatgctacacatattttaaaggccgccaattttgccaaggctaaaagaacaaggattctagagaaggaagtacttaaggaaactcatgaaaaagttcagggtggatttggtaaatatcagggtacatgggtcccactgaacatagcgaaacaactggcagaaaaatttagtgtctacgatcagctgaaaccgttgttcgactttacgcaaacagatgggtctgcttctccacctcctgctccaaaacatcaccatgcctcgaaggtggataggaaaaaggctattagaagtgcaagtacttccgcaattatggaaacaaaaagaaacaacaagaaagccgaggaaaatcaatttcaaagcagcaaaatattgggaaatcccacggctgcaccaaggaaaagaggtagaccggtaggatctacgaggggaagtaggcggaagttaggtgtcaatttacaacgttctcaaagtgatatgggatttcctagaccggcgataccgaattcttcaatatcgacaacgcaacttccctctattagatccaccatgggaccacaatcccctacattgggtattctggaagaagaaaggcacgattctcgacagcagcagccgcaacaaaataattctgcacagttcaaagaaattgatcttgaggacggcttatcaagcgatgtggaaccttcacaacaattacaacaagtttttaatcaaaatactggatttgtaccccaacaacaatcttccttgatacagacacagcaaacagaatcaatggccacgtccgtatcttcctctccttcattacctacgtcaccgggcgattttgccgatagtaatccatttgaagagcgatttcccggtggtggaacatctcctattatttccatgatcccgcgttatcctgtaacttcaaggcctcaaacatcggatattaatgataaagttaacaaatacctttcaaaattggttgattattttatttccaatgaaatgaagtcaaataagtccctaccacaagtgttattgcacccacctccacacagcgctccctatatagatgctccaatcgatccagaattacatactgccttccattgggcttgttctatgggtaatttaccaattgctgaggcgttgtacgaagccggaacaagtatcagatcgacaaattctcaaggccaaactccattgatgagaagttccttattccacaattcatacactagaagaactttccctagaattttccagctactgcacgagaccgtatttgatatcgattcgcaatcacaaacagtaattcaccatattgtgaaacgaaaatcaacaacaccttctgcagtttattatcttgatgttgtgctatctaagatcaaggatttttccccacagtatagaattgaattacttttaaacacacaagacaaaaatggcgataccgcacttcatattgcttctaaaaatggagatgttgttttttttaatacactggtcaaaatgggtgcattaactactatttccaataaggaaggattaaccgccaatgaaataatgaatcaacaatatgagcaaatgatgatacaaaatggtacaaatcaacatgtcaattcttcaaacacggacttgaatatccacgttaatacaaacaacattgaaacgaaaaatgatgttaattcaatggtaatcatgtcgcctgtttctccttcggattacataacctatccatctcaaattgccaccaatatatcaagaaatattccaaatgtagtgaattctatgaagcaaatggctagcatatacaacgatcttcatgaacagcatgacaacgaaataaaaagtttgcaaaaaactttaaaaagcatttctaagacgaaaatacaggtaagcctaaaaactttagaggtattgaaagagagcagtaaagatgaaaacggcgaagctcagactaatgatgacttcgaaattttatctcgtctacaagaacaaaatactaagaaattgagaaaaaggctcatacgatacaaacggttgataaaacaaaagctggaatacaggcaaacggttttattgaacaaattaatagaagatgaaactcaggctaccaccaataacacagttgagaaagataataatacgctggaaaggttggaattggctcaagaactaacgatgttgcaattacaaaggaaaaacaaattgagttccttggtgaagaaatttgaagacaatgccaagattcataaatatagacggattatcagggaaggtacggaaatgaatattgaagaagtagatagttcgctggatgtaatactacagacattgatagccaacaataataaaaataagggcgcagaacagatcatcacaatctcaaacgcgaatagtcatgca

SEQ ID NO: 44 - DNA, 324 bp, Artificial Sequence
  Description of Artificial Sequence Synthetic polynucleotide
agcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcc

SEQ ID NO: 45 - DNA, 987 bp, Artificial Sequence
  Description of Artificial Sequence Synthetic polynucleotide
gacatagttatgactcaaaccccgctttccctcccagtctcactgggggatcaagcgtccatctcatgccgctcttcacagagtattgtgcattctaacggtaacacatacctggaatggtatttgcaaaagccaggtcaaagcccaaagcttctcatctataaggtttcaaataggttttctggcgtcccagatcgattctccgggagtgggtctggtactgattttactcttaagatatcaagagtcgaggccgaggacttgggggtctattactgtttccaagggagccacgttccatatacttttgggggtgggacaaaactggaaataaaacgagggggcggagggtccggaggaggggggagtggcggaggagggtcaggtggcggaggatcccaggtgcagttgcaacagtcaggtccagaattggttaaacctggcgcgtctgtaaaaatgtcctgtaaagcgtccggatacacgtttacgagttacgttatgcactgggtgaaacagaaaccggggcagggcctggaatggatcgggtttatcaacttatacaacgatggaacaaagtacaatgaaaagtttaaaggcaaagccacgttgacttcagataaaagctcatcaactgcatatatggagctgtcatctcttacttccaaggatagcgcggtttattactgtgctcgggattattatggaagcagatggtttgactattggggacaagggacgacattgactgtatctagcggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaaggtggtggtagcggtggtggtactagtcccaagaagaagcgcaaggtg

SEQ ID NO: 46 - DNA, 189 bp, Artificial Sequence
  Description of Artificial Sequence Synthetic polynucleotide
gctacagtgaaatttaagtataagggggaggagaaggaagtggatatctccaagatcaagaaggtgtggcgcgtagggaaaatgatttcttttacttatgacgagggtggggggaagaccggacggggagccgtgtcagagaaagacgcccccaaggagctcctgcagatgctcgagaagcagaaaaaa

SEQ ID NO: 47 - DNA, 1008 bp, Artificial Sequence
  Description of Artificial Sequence Synthetic polynucleotide
agccttggaacaggaaatcggtgtgtcaagggggactcattgagcctcaaaggggagacagtaaatgattgtcacgcggaaatcataagtcgacggggcttcattcgatttctctacagcgaattgatgaaatacaactctcagacggcaaaagatagcatattcgaacctgcgaaagggggggagaagctccaaatcaagaagaccgtcagttttcacctttatatcagtaccgcaccctgcggtgacggcgcgcttttcgacaagagttgttcagaccgcgcaatggaatccacggaaagcagacattatccagtctttgagaatccgaaacagggcaaactccggacaaaagtcgaaaatggtcagggcacgatccccgttgagtcttcagatatcgttcccacctgggacgggattagactcggagagaggctccggacgatgagctgttcagataagatcctgcgatggaatgtcctgggcttgcaaggcgcgctgttgacacactttcttcagccaatttacctcaaatcagtcactctcggctacctcttttcacaagggcatctcacccgggccatttgttgtcgcgtgacaagggacggttccgcttttgaggacgggcttcgccatcccttcatagtaaatcaccccaaggtcggacgagtctcaatttacgactccaaacggcaatcaggaaagactaaagaaacgtctgtcaactggtgtctggctgatggctacgatcttgaaatacttgacgggacccgaggaaccgtcgacggccccaggaacgagcttagcagggtaagtaagaaaaatatattcctcctcttcaagaaactttgttcatttcgatataggcgcgacctgttgcgactgagctacggcgaggccaagaaggcggcgcgcgactacgagaccgccaagaattatttcaaaaagggactcaaggatatgggctatggaaattggatttccaaaccgcaagaggaaaagaatttc

SEQ ID NO: 48 - DNA, 1155 bp, Artificial Sequence
  Description of Artificial Sequence Synthetic polynucleotide
cagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctatacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacg

SEQ ID NO: 49 - DNA, 684 bp, Artificial Sequence
  Description of Artificial Sequence Synthetic polynucleotide
agcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctgaag

SEQ ID NO: 50 - DNA, 591 bp, Artificial Sequence
  Description of Artificial Sequence Synthetic polynucleotide
gacagtctgttgatgaatcgccgcaaatttttgtatcagttcaaaaatgtgcgttgggccaagggccgccgcgaaacatacctctgttatgtagtgaaacgtcgtgatagcgcaacatcattcagcctggacttcggatacctgcgcaacaaaaacggttgccacgtggagttgctgttcctgcgttacatctcagattgggatcttgatccgggccgttgttaccgtgtgacctggttcacatcgtggtccccgtgctatgattgcgcccgtcacgttgcggattttttacgtggtaacccgaatttgagcctgcgcatttttacagcgcgtctgtatttttgcgaagaccgtaaggcggaaccggaaggtctgcgtcgtttgcatcgcgcgggggtacagatcgctatcatgacctttaaagattatttttactgctggaacacctttgtggaaaaccatgaacgcacgtttaaagcgtgggaaggcctccacgaaaattcggtacgtctgtcgcgtcagctgcgccgtatcttactgccgctgtatgaggtcgatgatctgcgcgacgcctttcgtaccttgggcctg